(19) United States
(12) Patent Application Publication
(10) Pub. No.: US 2015/0352230 A1
(43) Pub. Date: Dec. 10, 2015

(54) SYNTHESIS AND ISOLATION OF DENDRIMER BASED IMAGING SYSTEMS

(71) Applicant: THE REGENTS OF THE UNIVERSITY OF MICHIGAN, Ann Arbor, MI (US)

(72) Inventors: Douglas Gurnett Mullen, Ann Arbor, MI (US); James R. Baker, JR., Ann Arbor, MI (US); Mark M. Banaszak Holl, Ann Arbor, MI (US); Baohua Huang, Ann Arbor, MI (US); Casey Dougherty, Ann Arbor, MI (US); Jack Ball, Ann Arbor, MI (US)

(21) Appl. No.: 14/760,388

(22) PCT Filed: Dec. 30, 2013
(86) PCT No.: PCT/US2013/078278

§ 371 Date: Jul. 10, 2015

Related U.S. Application Data
(60) Provisional application No. 61/751,610, filed on Jan. 11, 2013.

(51) Int. Cl. A61K 49/00; C07K 17/08

(52) U.S. Cl. CPC A61K 49/0054; C07K 17/08; A61K 49/0058; A61K 49/0041; A61K 49/0093

(57) ABSTRACT

The present invention relates to novel methods of synthesis and isolation of antibodies conjugated with modular dendrimer nanoparticles. In particular, the present invention is directed to antibodies conjugated with novel modular dendrimer nanoparticles having precise numbers of imaging agents, methods of synthesizing the same, compositions comprising such antibodies conjugated with such modular dendrimer nanoparticles, as well as systems and methods utilizing the conjugates (e.g., in imaging settings) (e.g., in diagnostic and/or therapeutic settings) (e.g., for the delivery of therapeutics, imaging, and/or targeting agents).

### FIELD OF THE INVENTION

The present invention relates to novel methods of synthesis and isolation of antibodies conjugated with modular dendrimer nanoparticles. In particular, the present invention is directed to antibodies conjugated with novel modular dendrimer nanoparticles having precise numbers of imaging agents, methods of synthesizing the same, compositions comprising such antibodies conjugated with such modular dendrimer nanoparticles, as well as systems and methods utilizing the conjugates (e.g., in imaging settings) (e.g., in diagnostic and/or therapeutic settings) (e.g., for the delivery of therapeutics, imaging, and/or targeting agents).

### BACKGROUND OF THE INVENTION

Antibody reagents labeled with molecular tags such as fluorescent dyes are essential tools for medical researchers studying biological processes, and for physicians diagnosing disease and monitoring the administration of therapy. Despite the clear success of labeled antibodies in scientific and medical applications, further progress in the field is limited by current technological paradigms that offer poor control over the number and positioning of dyes conjugated to each antibody (see, e.g., Hofer, T.; et al., Biochemistry 2009, 48, (50), 12047-12057; Vira, S.; et al., Analytical Biochemistry 2010, 402, (2), 146-150; Tadatsu, Y.; et al., The journal of medical investigation: JMI 2006, 53, (1-2), 52-60). As a result, labeled antibodies are neither highly quantitative nor optimally sensitive. In addition, labeled antibodies show high levels of batch-to-batch variability.

Improved imaging techniques are needed.

### SUMMARY

Embodiments of the present invention provide solutions to such problems. For example, embodiments of the present invention provide compositions comprising antibodies conjugated with dendrimer nanoparticles attached to precise numbers of dye agents. In addition, embodiments of the present invention provide methods for generating/synthesizing such compositions. In addition, embodiments of the present invention provide methods for using such compositions.

For example, for clinical applications, the consistency and reliability of reagents is paramount, and antibodies conjugated with such modular dendrimer nanodevices having precise numbers of imaging agents greatly reduces the risk of incorrect diagnoses as the result of reagent variability. In addition, some clinical assays, such as those for AIDS, require multi-time point measurements and thus multiple lots of the antibody reagent; these inter-batch measurements are more reliable with antibodies conjugated with such modular dendrimer nanodevices having precise numbers of imaging agents, due to batch-to-batch consistency. Finally, because of the high dye loadings and increased sensitivity with antibodies conjugated with such modular dendrimer nanodevices having precise numbers of imaging agents, earlier detection of diseases and pre-disease states is facilitated, leading to improved treatment outcomes.

In addition, antibodies conjugated with such modular dendrimer nanodevices having precise numbers of imaging agents provide additional benefits through increased efficiency in the manufacturing process, as every antibody can be labeled using the same method. For example, even if reagent manufacturers only used antibodies conjugated with such modular dendrimer nanodevices having precise numbers of imaging agents to replace current repertoire of labeled antibodies, antibodies conjugated with such modular dendrimer nanodevices having precise numbers of imaging agents permits the accomplishment more easily and with fewer resources. In addition, due to the modularity of the antibodies conjugated with such modular dendrimer nanodevices having precise numbers of imaging agents with respect to both imaging agents and number of imaging agents, manufacturers have the option to easily conjugate any of a wide range of dyes—in different defined quantities—using the same universal reaction scheme.

Accordingly, the present invention relates to novel methods of synthesis and isolation of antibodies conjugated with modular dendrimer nanoparticles. In particular, the present invention is directed to antibodies conjugated with novel modular dendrimer nanoparticles having precise numbers of imaging agents, methods of synthesizing the same, compositions comprising such antibodies conjugated with such modular dendrimer nanoparticles, as well as systems and methods utilizing the conjugates (e.g., in imaging settings) (e.g., in diagnostic and/or therapeutic settings) (e.g., for the delivery of therapeutics, imaging, and/or targeting agents). In certain embodiments, the present invention provides compositions comprising a plurality of antibodies having a precise number of imaging agents. The present invention is not limited to particular embodiments pertaining to a plurality of antibodies having a precise number of imaging agents.

In some embodiments, each of the antibodies within the plurality of antibodies are the same antibody. There is no limitation regarding the type or kind of antibody that may be used within such a plurality of antibodies. In some embodiments, for example, any of the antibodies recited in Tables 1 and 2 may be used. In some embodiments, the antibody is a monoclonal antibody. In some embodiments, the antibody is a polyclonal antibody.

In some embodiments, each of the plurality of antibodies are conjugated with two modular dendrimer nanoparticles. In some embodiments, each of the plurality of antibodies have an antibody Fc region, wherein the conjugation between the antibodies and the modular dendrimer nanoparticles occurs at the antibody Fc region. In some embodiments, the conjugation at the antibody Fc region occurs via a 1,3-dipolar cycloaddition reaction.

There is no limitation regarding the modular dendrimer nanoparticles. In some embodiments, approximately 70% or higher (e.g., approximately 60% or higher, 63% or higher, 65%, 68%, 70%, 72%, 75%, 80%, 81%, 83%, 85%, 90%, 92%, 95%, 97%, 98%, 99%, 99.999%, etc.) of the modular dendrimer nanoparticles are conjugated with a precise number and kind of imaging agents. In some embodiments, the conjugation between the imaging agents and the dendrimer occurs via imaging agent conjugation ligands (e.g., an alkene group, a thiol group, a dieneophile group, and a diene group) positioned on the dendrimers.

There are no limits regarding the number of imaging agents conjugated with the modular dendrimer nanoparticle. In some embodiments, the number of imaging agents is between 1 and 8.

There are no limits regarding the type or kind of imaging agent. In some embodiments, the imaging agent is selected from the group consisting of Alexa Fluor 350 (blue), Alexa Fluor 405 (violet), Alexa Fluor 430 (green), Alexa Fluor 488 (cyan-green), Alexa Fluor 500 (green), Alexa Fluor 514 (green), Alexa Fluor 532 (green), Alexa Fluor 546 (yellow), Alexa Fluor 555 (yellow-green), Alexa Fluor 568 (orange), Alexa Fluor 594 (orange-red), Alexa Fluor 610 (red), Alexa Fluor 633 (red), Alexa Fluor 647 (red), Alexa Fluor 660 (red), Alexa Fluor 680 (red), Alexa Fluor 700 (red), Alexa Fluor 750 (red), fluorescein isothiocyanate (FITC), 6-TAMARA, acridine orange, cis-parinaric acid, Hoechst 33342, Brilliant Violet™ 421, BD Horizon™ V450, Pacific Blue™, AmCyan, phycoerythrin (PE), Brilliant Violet™ 605, BD Horizon™ PE-CF594, PI, 7-AAD, allophycocyanin (APC), PE-Cy™ 5, PerCP, PerCP-Cy™ 5.5, PE-Cy™ 7, APC-Cy7, BD APC-H7, Texas Red, Lissamine Rhodamine B, X-Rhodamine, TRITC, Cy2, Cy3, Cy3B, Cy3.5, Cy5.5, Cy7, BODIPY-FL, FluorX™, TruRed, Red 613, NMD, Lucifer yellow, Pacific Orange, Pacific Blue, Cascade Blue, Methoxycoumarin, coumarin, hydroxycoumarin, aminocoumarin, 3-azidocoumarin, DyLight 350, DyLight 405, DyLight 488, DyLight® 550, DyLight 594, DyLight 633, DyLight® 650, DyLight 680, DyLight 755, DyLight 800, Tracy 645, Tracy 652, Atto 488, Atto 520, Atto 532, Atto Rho6G, Atto 550, Atto 565, Atto 590, Atto 594, Atto 633, Atto Rho11, Atto Rho14, Atto 647, Atto 647N, Atto 655, Atto 680, Atto 700, CF™ 350, CF™ 405S, CF™ 405M, CF™ 488A, CF™ 543, CF™ 555, CF™ 568, CF™ 594, CF™ 620R, CF™ 633, CF™ 640R, CF™ 647, CF™ 660, CF™ 660R, CF™ 680, CF™ 680R, CF™ 750, CF™ 770, and CF™ 790.

In some embodiments, the imaging agent is a mass-spec label selected from the group consisting of 139La, 141Pr, 142Nd, 143Nd, 144Nd, 145Nd, 146Nd, 147Sm, 148Nd, 149Sm, 150Nd, 151Eu, 152Sm, 153Eu, 154Sm, 156Gd, 158Gd, 159Tb, 160Gd, 162Dy, 164Dy, 165Ho, 166Er, 167Er, 168Er, 169Tm, 170Er, 171Yb, 172Yb, 174Yb, 175Lu, and 176Yb. In such embodiments wherein the imaging agent is a mass-spec label, its detection is accomplished with through mass-spectrometry.

In some embodiments, the modular dendrimer nanoparticle is conjugated with one or more additional functional groups selected from the group consisting of therapeutic agents, targeting agents, and trigger agents.

The modular dendrimer nanoparticles are not limited to a particular type of dendrimer. In some embodiments, the modular dendrimer nanoparticles comprise PAMAM dendrimers. In some embodiments, the dendrimers within the plurality of modular dendrimer nanoparticles have terminal branches, wherein the terminal branches comprise a blocking agent. In some embodiments, the blocking agent comprises an acetyl group.

In certain embodiments, the present invention provides compositions comprising a plurality of modular dendrimer nanoparticles, wherein approximately 70% or higher (e.g., approximately 60% or higher, 63% or higher, 65%, 68%, 70%, 72%, 75%, 80%, 81%, 83%, 85%, 90%/o, 92%, 95%, 97%, 98%, 99%, 99.999%/o, etc.) of the plurality of modular dendrimer nanoparticles have a precise number of imaging agent conjugation ligands.

The compositions are not limited to a particular type of imaging agent conjugation ligand. In some embodiments, the imaging agent conjugation ligand is selected from the group consisting of an alkene group, a thiol group, a dieneophile group, and a diene group. In some embodiments, the imaging agent conjugation ligand is configured for attachment with attachment ligands complexed with imaging agents.

In some embodiments, each of the plurality of modular dendrimer nanoparticles further comprise an antibody conjugation ligand. The compositions are not limited to a particular type of antibody conjugation ligand. In some embodiments, the antibody conjugation ligand is selected from the group consisting of a cyclooctyne group, a fluorinated cyclooctyne group, and an alkyne group. In some embodiments, the antibody conjugation ligand is configured to facilitate conjugation with another chemical group via click chemistry.

In some embodiments, the imaging agent conjugation ligands are conjugated with imaging agents. The compositions are not limited to a particular type of imaging agent.

In some embodiments, the imaging agents are selected from the group consisting of Alexa Fluor 350 (blue), Alexa Fluor 405 (violet), Alexa Fluor 430 (green), Alexa Fluor 488 (cyan-green), Alexa Fluor 500 (green), Alexa Fluor 514 (green), Alexa Fluor 532 (green), Alexa Fluor 546 (yellow), Alexa Fluor 555 (yellow-green), Alexa Fluor 568 (orange), Alexa Fluor 594 (orange-red), Alexa Fluor 610 (red), Alexa Fluor 633 (red), Alexa Fluor 647 (red), Alexa Fluor 660 (red), Alexa Fluor 680 (red), Alexa Fluor 700 (red), Alexa Fluor 750 (red), fluorescein isothiocyanate (FITC), 6-TAMARA, acridine orange, cis-parinaric acid, Hoechst 33342, Brilliant Violet™ 421, BD Horizon™ V450, Pacific Blue™, AmCyan, phycoerythrin (PE), Brilliant Violet™ 605, BD Horizon™ PE-CF594, PI, 7-AAD, allophycocyanin (APC), PE-Cy™ 5, PerCP, PerCP-Cy™ 5.5, PE-Cy™ 7, APC-Cy7, BD APC-H7, Texas Red, Lissamine Rhodamine B, X-Rhodamine, TRITC, Cy2, Cy3, Cy3B, Cy3.5, Cy5.5, Cy7, BODIPY-FL, FluorX™, TruRed, Red 613, NMD, Lucifer yellow, Pacific Orange, Pacific Blue, Cascade Blue, Methoxycoumarin, coumarin, hydroxycoumarin, aminocoumarin, 3-azidocoumarin, DyLight 350, DyLight 405, DyLight 488, DyLight™ 550, DyLight 594, DyLight 633, DyLight™ 650, DyLight 680, DyLight 755, DyLight 800, Tracy 645, Tracy 652, Atto 488, Atto 520, Atto 532, Atto Rho6G, Atto 550, Atto 565, Atto 590, Atto 594, Atto 633, Atto Rho11, Atto Rho14, Atto 647, Atto 647N, Atto 655, Atto 680, Atto 700, CF™ 350, CF™ 405S, CF™ 405M, CF™ 488A, CF™ 543, CF™ 555, CF™ 568, CF™ 594, CF™ 620R, CF™ 633, CF™ 640R, CF™ 647, CF™ 660, CF™ 660R, CF™ 680, CF™ 680R, CF™ 750, CF™ 770, and CF™ 790.

In some embodiments, the imaging agent is a mass-spec label selected from the group consisting of 139La, 141Pr, 142Nd, 143Nd, 144Nd, 145Nd, 146Nd, 147Sm, 148Nd, 149Sm, 150Nd, 151Eu, 152Sm, 153Eu, 154Sm, 156Gd, 158Gd, 159Tb, 160Gd, 162Dy, 164Dy, 165Ho, 166Er, 167Er, 168Er, 169Tm, 170Er, 171Yb, 172Yb, 174Yb, 175Lu, and 176Yb. In such embodiments wherein the imaging agent is a mass-spec label, its detection is accomplished with through mass-spectrometry.

In some embodiments, the antibody conjugation ligand is conjugated with an antibody. In some embodiments, the conjugation with an antibody is at the Fc region of the antibody. In some embodiments, the conjugation with an antibody occurs via a 1,3-dipolar cycloaddition reaction.

The compositions are not limited to a particular type of antibody. In some embodiments, the antibody is a monoclonal antibody. In some embodiments, the antibody is a polyclonal antibody. In some embodiments, the antibody is an antibody selected from the group consisting of the antibodies shown in Tables 1 and 2.

In some embodiments, the plurality of modular dendrimer nanoparticles are conjugated with one or more additional functional groups selected from the group consisting of therapeutic agents, targeting agents, and trigger agents.

The modular dendrimer nanoparticles are not limited to a particular type of dendrimer. In some embodiments, the modular dendrimer nanoparticles comprise PAMAM dendrimers. In some embodiments, the dendrimers within the plurality of modular dendrimer nanoparticles have terminal branches, wherein the terminal branches comprise a blocking agent. In some embodiments, the blocking agent comprises an acetyl group.

In certain embodiments, the present invention provides methods for generating pluralities of modular dendrimer nanoparticles wherein approximately 70% or more of the batches of modular dendrimer nanoparticles have a precise number of imaging agent conjugation ligands. In some embodiments, the methods comprise conjugating imaging agent conjugation ligands with a plurality of dendrimer nanoparticles; and separating the plurality of dendrimer nanoparticles conjugated with the imaging agent conjugation ligands into pluralities based upon the number of imaging agent conjugation ligands conjugated to the dendrimer nanoparticles, wherein approximately 70% or higher (e.g., approximately 60% or higher, 63% or higher, 65%, 68%, 70%, 72%, 75%, 80%, 819%, 83%, 85%, 90%, 92%, 95%, 97%, 98%, 99%, 99.999%, etc.) of each batch of modular dendrimer nanoparticles have a precise number of imaging agent conjugation ligands.

The methods are not limited to a particular separation technique and/or method. In some embodiments, such separation involves application of reverse phase HPLC to yield a subpopulation of pluralities based upon the number of imaging agent conjugation ligands conjugated to the dendrimer nanoparticles indicated by a chromatographic trace, and applying a peak fitting analysis to the chromatographic trace to identify pluralities of modular dendrimer nanoparticles wherein approximately 70% or more of the pluralities of modular dendrimer nanoparticles have a precise number of imaging agent conjugation ligands. In some embodiments, the reverse phase HPLC is performed using silica gel media comprising a carbon moiety, the carbon moiety ranging from C3 to C8. In some embodiments, the reverse phase HPLC is performed using C5 silica gel media. In some embodiments, the reverse phase HPLC is conducted using a mobile phase for elution of the ligand-conjugated dendrimers, wherein the mobile phase comprises a linear gradient beginning with 100:0 (v/v) water:acetonitrile and ending with 20:80 (v/v) water:acetonitrile. In some embodiments, the reverse phase HPLC is conducted using a mobile phase for elution of the ligand-conjugated dendrimers, wherein the mobile phase comprises a linear gradient beginning with 100:0 (v/v) water:isopropanol and ending with 20:80 (v/v) water:isopropanol. In some embodiments, the gradient is applied at a flow rate of 1 ml/min. In some embodiments, the gradient is applied at a flow rate of 10 ml/min. In some embodiments, the peak fitting analysis is performed using a Gaussian fit with an exponential decay tail.

The methods are not limited to a particular type of imaging agent conjugation ligand. In some embodiments, the imaging agent conjugation ligand is selected from the group consisting of an alkene group, a thiol group, a dieneophile group, and a diene group. In some embodiments, the imaging agent conjugation ligand is configured for attachment with attachment ligands complexed with imaging agents.

In some embodiments, the methods further comprise conjugating an antibody conjugation ligand with one or more of the batches of modular dendrimer nanoparticles have a precise number of imaging agent conjugation ligands. The methods are not limited to a particular type of antibody conjugation ligand. In some embodiments, the antibody conjugation ligand is selected from the group consisting of a cyclooctyne group, a fluorinated cyclooctyne group, and an alkyne group. In some embodiments, the antibody conjugation ligand is configured to facilitate conjugation with another chemical group via click chemistry.

In some embodiments, the methods further comprise conjugating imaging agents with one or more of the batches of modular dendrimer nanoparticles having a precise number of imaging agent conjugation ligands, wherein the conjugating occurs between the imaging agents and the imaging agent conjugation ligands. The methods are not limited to a particular type of imaging agent.

In some embodiments, the imaging agents are selected from the group consisting of Alexa Fluor 350 (blue), Alexa Fluor 405 (violet), Alexa Fluor 430 (green), Alexa Fluor 488 (cyan-green), Alexa Fluor 500 (green), Alexa Fluor 514 (green), Alexa Fluor 532 (green), Alexa Fluor 546 (yellow), Alexa Fluor 555 (yellow-green), Alexa Fluor 568 (orange), Alexa Fluor 594 (orange-red), Alexa Fluor 610 (red), Alexa Fluor 633 (red), Alexa Fluor 647 (red), Alexa Fluor 660 (red), Alexa Fluor 680 (red), Alexa Fluor 700 (red), Alexa Fluor 750 (red), fluorescein isothiocyanate (FITC), 6-TAMARA, acridine orange, cis-parinaric acid, Hoechst 33342, Brilliant Violet™ 421, BD Horizon™ V450, Pacific Blue™, AmCyan, phycoerythrin (PE), Brilliant Violet™ 605, BD Horizon™ PE-CF594, PI, 7-AAD, allophycocyanin (APC), PE-Cy™ 5, PerCP, PerCP-Cy™ 5.5, PE-Cy™ 7, APC-Cy7, BD APC-H7, Texas Red, Lissamine Rhodamine B, X-Rhodamine, TRITC, Cy2, Cy3, Cy3B, Cy3.5, Cy5.5, Cy7, BODIPY-FL, FluorX™, TruRed, Red 613, NMD, Lucifer yellow, Pacific Orange, Pacific Blue, Cascade Blue, Methoxycoumarin, coumarin, hydroxycoumarin, aminocoumarin, 3-azidocoumarin, DyLight 350, DyLight 405, DyLight 488, DyLight™ 550, DyLight 594, DyLight 633, DyLight™ 650, DyLight 680, DyLight 755, DyLight 800, Tracy 645, Tracy 652, Atto 488, Atto 520, Atto 532, Atto Rho6G, Atto 550, Atto 565, Atto 590, Atto 594, Atto 633, Atto Rho11, Atto Rho14, Atto 647, Atto 647N, Atto 655, Atto 680, Atto 700, CF™ 350, CF™ 405S, CF™ 405M, CF™ 488A, CF™ 543, CF™ 555, CF™ 568, CF™ 594, CF™ 620R, CF™ 633, CF™ 640R, CF™ 647, CF™ 660, CF™ 660R, CF™680, CF™ 680R, CF™ 750, CF™ 770, and CF™ 790.

In some embodiments, the imaging agent is a mass-spec label selected from the group consisting of 139La, 141Pr, 142Nd, 143Nd, 144Nd, 145Nd, 146Nd, 147Sm, 148Nd, 149Sm, 150Nd, 151Eu, 152Sm, 153Eu, 154Sm, 156Gd, 158Gd, 159Tb, 160Gd, 162Dy, 164Dy, 165Ho, 166Er, 167Er, 168Er, 169Tm, 170Er, 171Yb, 172Yb, 174Yb, 175Lu, and 176Yb.

In some embodiments, the methods further comprise conjugating two of the modular dendrimer nanoparticles having a precise number of imaging agent conjugation ligands from one or more of the batches with an antibody. In some embodiments, the conjugation with an antibody is at the Fc region of the antibody. In some embodiments, the conjugation with an antibody occurs via a 1,3-dipolar cycloaddition reaction.

The methods are not limited to a particular type of antibody. In some embodiments, the antibody is a monoclonal antibody. In some embodiments, the antibody is a polyclonal antibody. In some embodiments, the antibody is an antibody selected from the group consisting of the antibodies shown in Tables 1 and 2.

In certain embodiments, the present invention provides methods of imaging, comprising administering to a sample one or more of the plurality of antibodies conjugated with two modular dendrimer nanoparticles having a precise number and kind of imaging agents, wherein the antibodies are capable of binding a cell surface antigens associated with the antibodies, and wherein upon binding with the cell surface antigens associated with the antibodies the imaging agents are detected. In some embodiments, the sample is a cell sample. In some embodiments, the sample is within a living subject.

In certain embodiments, the present invention provides methods of imaging a tissue region of interest in a subject, comprising administering to the subject one or more antibodies conjugated with two modular dendrimer nanoparticles having a precise number and kind of imaging agents, wherein the one or more antibodies bind to the tissue region of interest, and wherein upon binding with the tissue region of interest the imaging agents are detected. In some embodiments, the subject is a living mammal. In some embodiments, the imaging is used to characterize the tissue region of interest. In some embodiments, the characterizing is diagnosing the presence or absence of a disorder.

In certain embodiments, the present invention provides methods of imaging a tissue region of interest in a subject, comprising obtaining a sample from a subject, wherein the sample comprises a tissue region of interest in the subject, administering to the sample one or more antibodies conjugated with two modular dendrimer nanoparticles having a precise number and kind of imaging agents, wherein the one or more antibodies bind to the tissue region of interest, and wherein upon binding with the tissue region of interest the imaging agents are detected. In some embodiments, the subject is a living mammal. In some embodiments, imaging is used to characterize the tissue region of interest. In some embodiments, the characterizing is diagnosing the presence or absence of a disorder.

In certain embodiments, the present invention provides methods for imaging different antigens having varying abundance quantities in a manner wherein the detected imaging agent intensity is equated. For example, in some embodiments, different types of antigens have differing levels of in vivo or in vitro abundance. In such embodiments, antibodies directed to the higher abundance antigen are configured to be conjugated with modular dendrimer nanoparticles having fewer imaging agents (e.g., 2 imaging agents) than modular dendrimer nanoparticles conjugated with antibodies directed to the lower abundance antigen (e.g., 16 imaging agents). Such embodiments permit the equating of imaging agent intensity for antigens regardless of the abundance levels of such antigens.

Additional embodiments will be apparent to persons skilled in the relevant art based on the teachings contained herein.

### DESCRIPTION OF THE DRAWINGS

FIG. 1 shows an embodiment of the present invention having a dendrimer scaffold with an antibody conjugation ligand (orthogonal antibody conjugation linker) and an exact number of imaging agent conjugation ligands (dye attachment sites), and the subsequent attachment of imaging agents (dyes) to the imaging agent conjugation ligands on the dendrimer scaffold.

FIG. 2 shows an antibody conjugated with two modular dendrimer nanoparticles having a precise number of imaging agents (DLabel). As shown, the Fc region of the antibody is configured with an azide-modified C-termini.

FIG. 3 shows HPLC elution profiles of dendrimers with precise numbers of alkyne-terminated ligands isolated by Semi-Preparatory HPLC from the distribution of dendrimer-ligand species.

FIG. 4 shows imaging results for samples as described in Example 6.

### DEFINITIONS

To facilitate an understanding of the present invention, a number of terms and phrases are defined below:

As used herein, the term “subject” refers to any animal (e.g., a mammal), including, but not limited to, humans, non-human primates, rodents, and the like, which is to be the recipient of a particular treatment. Typically, the terms “subject” and “patient” are used interchangeably herein in reference to a human subject.

As used herein, the term “non-human animals” refers to all non-human animals including, but not limited to, vertebrates such as rodents, non-human primates, ovines, bovines, ruminants, lagomorphs, porcines, caprines, equines, canines, felines, aves, etc.

As used herein, the term “subject suspected of having cancer” refers to a subject that presents one or more symptoms indicative of a cancer (e.g., a noticeable lump or mass) or is being screened for a cancer (e.g., during a routine physical). A subject suspected of having cancer may also have one or more risk factors. A subject suspected of having cancer has generally not been tested for cancer. However, a “subject suspected of having cancer” encompasses an individual who has received a preliminary diagnosis (e.g., a CT scan showing a mass) but for whom a confirmatory test (e.g., biopsy and/or histology) has not been done or for whom the stage of cancer is not known. The term further includes people who once had cancer (e.g., an individual in remission). A “subject suspected of having cancer” is sometimes diagnosed with cancer and is sometimes found to not have cancer.

As used herein, the term “subject diagnosed with a cancer” refers to a subject who has been tested and found to have cancerous cells. The cancer may be diagnosed using any suitable method, including but not limited to, biopsy, x-ray, blood test, and the diagnostic methods of the present invention.

As used herein, the term “sample” is used in its broadest sense. In one sense, it is meant to include a specimen or culture obtained from any source, as well as biological and environmental samples. Biological samples may be obtained from animals (including humans) and encompass fluids, solids, tissues, and gases. Biological samples include blood products, such as plasma, serum and the like. Environmental samples include environmental material such as surface matter, soil, water, crystals and industrial samples. Such examples are not however to be construed as limiting the sample types applicable to the present invention.

As used herein, the term “drug” is meant to include any molecule, molecular complex or substance administered to an organism for diagnostic or therapeutic purposes, including medical imaging, monitoring, contraceptive, cosmetic, nutraceutical, pharmaceutical and prophylactic applications. The term “drug” is further meant to include any such molecule, molecular complex or substance that is chemically modified and/or operatively attached to a biologic or biocompatible structure.

As used herein, the term “purified” or “to purify” or “compositional purity” refers to the removal of components (e.g., contaminants) from a sample or the level of components (e.g., contaminants) within a sample. For example, unreacted moieties, degradation products, excess reactants, or byproducts are removed from a sample following a synthesis reaction or preparative method.

“Amino acid sequence” and terms such as “polypeptide” or “protein” are not meant to limit the amino acid sequence to the complete, native amino acid sequence associated with the recited protein molecule.

The term “native protein” as used herein to indicate that a protein does not contain amino acid residues encoded by vector sequences; that is, the native protein contains only those amino acids found in the protein as it occurs in nature. A native protein may be produced by recombinant means or may be isolated from a naturally occurring source.

As used herein the term “portion” when in reference to a protein (as in “a portion of a given protein”) refers to fragments of that protein. The fragments may range in size from four amino acid residues to the entire amino acid sequence minus one amino acid.

As used herein, the term “cell culture” refers to any in vitro culture of cells. Included within this term are continuous cell lines (e.g., with an immortal phenotype), primary cell cultures, transformed cell lines, finite cell lines (e.g., non-transformed cells), and any other cell population maintained in vitro.

As used herein, the term “eukaryote” refers to organisms distinguishable from “prokaryotes.” It is intended that the term encompass all organisms with cells that exhibit the usual characteristics of eukaryotes, such as the presence of a true nucleus bounded by a nuclear membrane, within which lie the chromosomes, the presence of membrane-bound organelles, and other characteristics commonly observed in eukaryotic organisms. Thus, the term includes, but is not limited to such organisms as fungi, protozoa, and animals (e.g., humans).

As used herein, the term “in vitro” refers to an artificial environment and to processes or reactions that occur within an artificial environment. In vitro environments can consist of, but are not limited to, test tubes and cell culture. The term “in vivo” refers to the natural environment (e.g., an animal or a cell) and to processes or reaction that occur within a natural environment.

The terms “test compound” and “candidate compound” refer to any chemical entity, pharmaceutical, drug, and the like that is a candidate for use to treat or prevent a disease, illness, sickness, or disorder of bodily function (e.g., cancer). Test compounds comprise both known and potential therapeutic compounds. A test compound can be determined to be therapeutic by screening using screening methods known in the art.

As used herein, the term “nanodevice” or “nanodevices” or “nanoparticle” or “nanoparticles” refer, generally, to compositions comprising dendrimers of the present invention. As such, a nanodevice or nanoparticle may refer to a composition comprising a dendrimer of the present invention that may contain one or more ligands, linkers, and/or functional groups (e.g., a therapeutic agent, a targeting agent, a trigger agent, an imaging agent) conjugated to the dendrimer.

As used herein, the term “degradable linkage,” when used in reference to a polymer refers to a conjugate that comprises a physiologically cleavable linkage (e.g., a linkage that can be hydrolyzed (e.g., in vivo) or otherwise reversed (e.g., via enzymatic cleavage). Such physiologically cleavable linkages include, but are not limited to, ester, carbonate ester, carbamate, sulfate, phosphate, acyloxyalkyl ether, acetal, and ketal linkages (See, e.g., U.S. Pat. No. 6,838,076). Similarly, the conjugate may comprise a cleavable linkage present in the linkage between the dendrimer and functional group, or, may comprise a cleavable linkage present in the polymer itself (See, e.g., U.S. Pat. App. Nos. 20050158273 and 20050181449).

A “physiologically cleavable” or “hydrolysable” or “degradable” bond is a bond that reacts with water (i.e., is hydrolyzed) under physiological conditions. The tendency of a bond to hydrolyze in water will depend not only on the general type of linkage connecting two central atoms but also on the substituents attached to these central atoms. Appropriate hydrolytically unstable or weak linkages include but are not limited to carboxylate ester, phosphate ester, anhydrides, acetals, ketals, acyloxyalkyl ether, imines, orthoesters, peptides and oligonucleotides.

An “enzymatically degradable linkage” means a linkage that is subject to degradation by one or more enzymes.

A “hydrolytically stable” linkage or bond refers to a chemical bond (e.g., typically a covalent bond) that is substantially stable in water (i.e., does not undergo hydrolysis under physiological conditions to any appreciable extent over an extended period of time). Examples of hydrolytically stable linkages include, but are not limited to, carbon-carbon bonds (e.g., in aliphatic chains), ethers, amides, urethanes, and the like.

As used herein, the term “NAALADase inhibitor” refers to any one of a multitude of inhibitors for the neuropeptidase NAALADase (N-acetylated-alpha linked acidic dipeptidase). Such inhibitors of NAALADase have been well characterized. For example, an inhibitor can be selected from the group comprising, but not limited to, those found in U.S. Pat. No. 6,011,021.

As used herein, an “NH₂-terminal blocking agent” is a functional group that prevents the reactivity of NH₂-terminal branches of dendrimers. Such blocking agents include but are not limited to acetyl groups. Blocking of NH₂-terminal dendrimers may be partial or complete.

As used herein, an “ester coupling agent” refers to a reagent that can facilitate the formation of an ester bond between two reactants. The present invention is not limited to any particular coupling agent or agents. Examples of coupling agents include but are not limited to 2-chloro-1-methylpyridium iodide and 4-(dimethylamino)pyridine, or dicyclohexylcarbodiimide and 4-(dimethylamino)pyridine or diethyl azodicarboxylate and triphenylphosphine or other carbodiimide coupling agent and 4-(dimethylamino)pyridine.

As used herein, the term “glycidolate” refers to the addition of a 2,3-dihydroxylpropyl group to a reagent using glycidol as a reactant. In some embodiments, the reagent to which the 2,3-dihydroxylpropyl groups are added is a dendrimer. In some embodiments, the dendrimer is a PAMAM dendrimer. Glycidolation may be used generally to add terminal hydroxyl functional groups to a reagent.

As used herein, the term “amino alcohol” or “amino-alcohol” refers to any organic compound containing both an amino and an aliphatic hydroxyl functional group (e.g., which may be an aliphatic or branched aliphatic or alicyclic or hetero-alicyclic compound containing an amino group and one or more hydroxyl(s)). The generic structure of an amino alcohol may be expressed as NH₂—R—(OH)ₘ wherein m is an integer, and wherein R comprises at least two carbon molecules (e.g., at least 2 carbon molecules, 10 carbon molecules, 25 carbon molecules, 50 carbon molecules).

As used herein, the term “one-pot synthesis reaction” or equivalents thereof, e.g., “1-pot”, “one pot”, etc., refers to a chemical synthesis method in which all reactants are present in a single vessel. Reactants may be added simultaneously or sequentially, with no limitation as to the duration of time elapsing between introduction of sequentially added reactants. In some embodiments, conjugation between a dendrimer (e.g., a terminal arm of a dendrimer) and a functional ligand is accomplished during a “one-pot” reaction. The term “one-pot synthesis reaction” or equivalents thereof, e.g., “1-pot”, “one pot”, etc., refers to a chemical synthesis method in which all reactants are present in a single vessel. Reactants may be added simultaneously or sequentially, with no limitation as to the duration of time elapsing between introduction of sequentially added reactants. In some embodiments, a one-pot reaction occurs wherein a hydroxyl-terminated dendrimer (e.g., HO-PAMAM dendrimer) is reacted with one or more functional ligands (e.g., a therapeutic agent, a pro-drug, a trigger agent, a targeting agent, an imaging agent) in one vessel, such conjugation being facilitated by ester coupling agents (e.g., 2-chloro-1-methylpyridinium iodide and 4-(dimethylamino)pyridine) (see, e.g., U.S. Patent App. No. 61/226,993).

As used herein, the term “solvent” refers to a medium in which a reaction is conducted. Solvents may be liquid but are not limited to liquid form. Solvent categories include but are not limited to nonpolar, polar, protic, and aprotic.

As used herein, the term “dialysis” refers to a purification method in which the solution surrounding a substance is exchanged over time with another solution. Dialysis is generally performed in liquid phase by placing a sample in a chamber, tubing, or other device with a selectively permeable membrane. In some embodiments, the selectively permeable membrane is cellulose membrane. In some embodiments, dialysis is performed for the purpose of buffer exchange. In some embodiments, dialysis may achieve concentration of the original sample volume. In some embodiments, dialysis may achieve dilution of the original sample volume.

As used herein, the term “precipitation” refers to purification of a substance by causing it to take solid form, usually within a liquid context. Precipitation may then allow collection of the purified substance by physical handling, e.g. centrifugation or filtration.

As used herein, the term “Baker-Huang dendrimer” or “Baker-Huang PAMAM dendrimer” refers to a dendrimer comprised of branching units of structure:

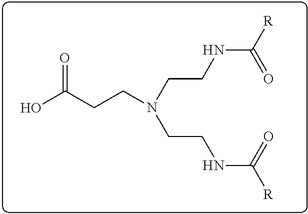

wherein R comprises a carbon-containing functional group (e.g., CF₃). In some embodiments, the branching unit is activated to its HNS ester. In some embodiments, such activation is achieved using TSTU. In some embodiments, EDA is added. In some embodiments, the dendrimer is further treated to replace, e.g., CF₃ functional groups with NH₂ functional groups; for example, in some embodiments, a CF₃-containing version of the dendrimer is treated with K₂CO₃ to yield a dendrimer with terminal NH₂ groups (for example, as shown in U.S. patent application Ser. No. 12/645,081). In some embodiments, terminal groups of a Baker-Huang dendrimer are further derivatized and/or further conjugated with other moieties. For example, one or more functional ligands (e.g., for therapeutic, targeting, imaging, or drug delivery function(s)) may be conjugated to a Baker-Huang dendrimer, either via direct conjugation to terminal branches or indirectly (e.g., through linkers, through other functional groups (e.g., through an OH— functional group)). In some embodiments, the order of iterative repeats from core to surface is amide bonds first, followed by tertiary amines, with ethylene groups intervening between the amide bond and tertiary amines. In preferred embodiments, a Baker-Huang dendrimer is synthesized by convergent synthesis methods.

As used herein, the term “click chemistry” refers to chemistry tailored to generate substances quickly and reliably by joining small modular units together (see. e.g., Kolb et al. (2001) Angewandte Chemie Intl. Ed. 40:2004-2011; Evans (2007) Australian J. Chem. 60:384-395; Carlmark et al. (2009) Chem. Soc. Rev. 38:352-362).

As used herein, the term “alkyne ligand” refers to a ligand bearing an alkyne functional group. In some embodiments, alkyne ligands further comprise an aromatic group.

As used herein, the term “azide ligand” refers to a ligand bearing an azide functional group. In some embodiments, azide ligands further comprise an aromatic group.

As used herein, the term “peak fitting analysis” refers to mathematical determination of the functional form of a curve in a chromatographic trace. In some embodiments, an HPLC trace is used. In some embodiments, a reverse phase HPLC trace is used. In some embodiments, software is used for peak fitting analysis (e.g., graphing software, image analysis software, data analysis software). In some embodiments, the Igor Pro software package is used. Functional forms applied to peaks may include but are not limited to Gaussian, double exponential, polynomial, Lorentzian, linear, exponential, power law, sine, log normal, Hill equation, sigmoid, or a combination thereof. In some embodiments, a Gaussian curve with an exponential decay tail is applied. Fitting peaks may be constrained or not constrained.

As used herein, the term “high performance liquid chromatography” or “high pressure liquid chromatography” or “HPLC” refers to techniques known in the art of macromolecule separation, quantification, and identification. HPLC is used to separate mixtures of molecules on the basis of inherent properties possessed by the molecules including but not limited to size, polarity, ligand affinity, hydrophobicity, and charge. In some embodiments, “reverse phase HPLC” (also referred to as “reversed phase HPLC”, “reverse-phase HPLC”, “reversed-phase HPLC”, “RPC” or “RP-HPLC”) may be used with methods, systems, and synthesis methods of the present invention. Reverse phase HPLC involves a non-polar stationary phase and an aqueous, moderately polar mobile phase. One common stationary phase is a silica which has been treated with RMe₂SiCl, where R is a straight chain alkyl group such as C₁₈H₃₇ or C₈H₁₇. The number of carbons in the straight chain alkyl group can vary (e.g., 2, 3, 4, 5, 6, 7, 8, greater than 8). With these stationary phases, retention time is longer for molecules which are more non-polar, while polar molecules elute more readily. Retention time can be increased by adding more water to the mobile phase; thereby making the affinity of the hydrophobic analyte for the hydrophobic stationary phase stronger relative to the now more hydrophilic mobile phase. Similarly, retention time can be decreased by adding more organic solvent to the eluent.

As used herein, the term “distribution” refers to the variance in the number of different ligands attached to a dendrimer within a population of dendrimers. For example, a dendrimer sample in which the average number of ligands attachments (ligand conjugates) is 5 may have a distribution of 0-10 (i.e., some proportion of the dendrimers in the population have no ligands attached, some proportion of the dendrimers in the population have 10 ligands attached, and other proportions have between 2 and 9 ligands attached.)

As used herein, the term “ligand” refers to any moiety covalently attached (e.g., conjugated) to a dendrimer branch. Some ligands may serve as “linkers” such that they intervene or are intended to intervene in the future between the dendrimer branch terminus and another more terminal ligand. Some ligands have functional utility for specific applications, e.g., for therapeutic, targeting, imaging, or drug delivery function(s). The terms “ligand” and “conjugate” may be used interchangeably.

As used herein, the term “inflammatory disease” refers to any disease characterized by inflammation of tissues or cells. Inflammatory diseases may be acute or chronic, and include but are not limited to eczema, inflammatory bowel disease, ulcerative colitis, multiple sclerosis, myocarditis, rheumatoid arthritis, asthma, psoriasis, ischemia/reperfusion injury, ulcerative colitis, necrotizing enterocolitis, pelvic inflammatory disease, empyema, pleurisy, pyelitis, pharyginitis, acne, urinary tract infection, Crohn disease, systemic lupus erythematosus, and acute respiratory distress syndrome.

As used herein, the term “rheumatoid arthritis” (RA) refers to a chronic systemic inflammatory disease of unknown cause that primarily affects the peripheral joints in a symmetric pattern. Common symptoms include but are not limited to fatigue, malaise, and morning stiffness. Extra-articular involvement of organs such as the skin, heart, lungs, and eyes can be significant. One of ordinary skill in the medical arts appreciate that RA causes joint destruction and thus often leads to considerable morbidity and mortality.

As used herein, the term “structural uniformity” refers to the number of ligand conjugations within a dendrimer device (e.g., dendrimer system, ligand-conjugated dendrimer). In a population of dendrimer compositions with 100% structural uniformity, for example, all dendrimer molecules bear the same number of ligands if one ligand type is present; or the same number of each type of ligand if different ligand types are present. As used herein, high structural uniformity does not preclude variances in dendrimer backbone and/or branches insofar as such variances do not impact the number of ligand attachments.

### DETAILED DESCRIPTION OF THE INVENTION

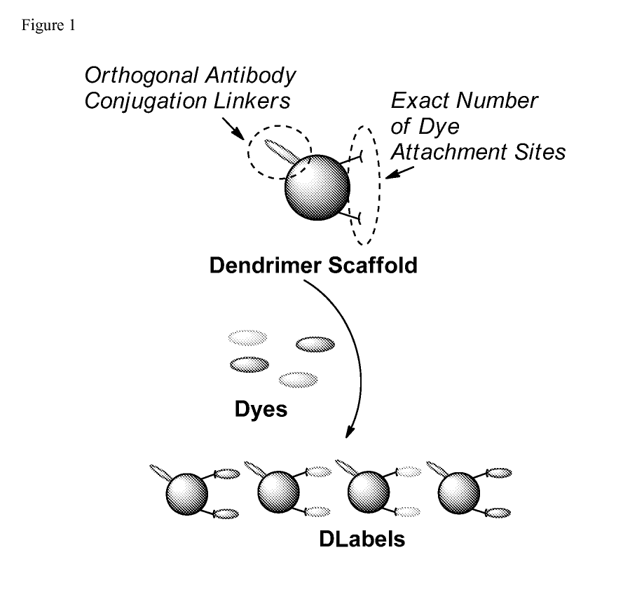

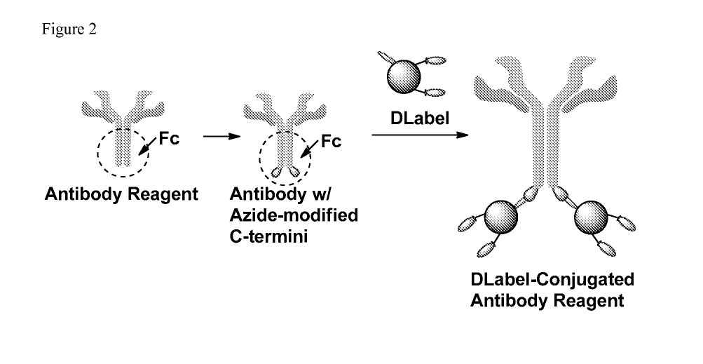

Embodiments of the present invention describe modular dendrimer nanoparticles with precise numbers of imaging agents (e.g., dye molecules) per particle and antibody conjugation ligands (see, e.g., FIG. 1). Such modular dendrimer nanoparticles with precise numbers of imaging agents (e.g., dye molecules) per particle and antibody conjugation ligands are not limited to particular uses. In some embodiments, such modular dendrimer nanoparticles with precise numbers of imaging agents (e.g., dye molecules) per particle and antibody conjugation ligands are used to label antibodies so as to generate antibodies labeled with a quantitative number of imaging agents (e.g., dye molecules) (see, e.g., FIG. 2).

The present invention is not limited to a particular method and/or technique for generating modular dendrimer nanoparticles and/or batches of modular dendrimer nanoparticles. In some embodiments, modular dendrimer nanoparticles having precisenumbers of imaging agent conjugation ligands are isolated (e.g., through HPLC isolation techniques) prior to conjugation with imaging agents (e.g., so as to ensure the generation of a batch of modular dendrimer nanoparticles having precise numbers of imaging agents conjugated to such imaging agent conjugation linkers). In some embodiments, the modular dendrimer nanoparticles are additionally complexed with an antibody conjugation ligand. In some embodiments, imaging agents (e.g., dyes) are conjugated to such modular dendrimer nanoparticles having precise numbers of imaging agent conjugation ligands. Such techniques ensure that a particular batch of modular dendrimer nanoparticles has a precise number of imaging agents (e.g., dyes). In some embodiments, batches of such modular dendrimer nanoparticles having a precise number of imaging agents (e.g., dyes) are complexed with particular antibodies, thereby generating batches of antibodies labeled with precise numbers of imaging agents (e.g., dyes).

The modular dendrimer nanoparticles are not limited to utilizing a particular type of dendrimer nanoparticle. Dendrimeric polymers have been described extensively (see, e.g., Tomalia, Advanced Materials 6:529 (1994); Angew, Chem. Int. Ed. Engl., 29:138 (1990)). Dendrimer polymers are synthesized as defined spherical structures typically ranging from 1 to 20 nanometers in diameter. Methods for manufacturing a G5 PAMAM dendrimer with a protected core are known (U.S. patent application Ser. No. 12/403,179). In preferred embodiments, the protected core diamine is NH₂—CH₂—CH₂—NHPG. Molecular weight and the number of terminal groups increase exponentially as a function of generation (the number of layers) of the polymer. In some embodiments of the present invention, half generation PAMAM dendrimers are used. For example, when an ethylenediamine (EDA) core is used for dendrimer synthesis, alkylation of this core through Michael addition results in a half-generation molecule with ester terminal groups; amidation of such ester groups with excess EDA results in creation of a full-generation, amine-terminated dendrimer (Majoros et al., Eds. (2008) Dendrimer-based Nanomedicine, Pan Stanford Publishing Pte. Ltd., Singapore, p. 42). Different types of dendrimers can be synthesized based on the core structure that initiates the polymerization process. In some embodiments, the PAMAM dendrimers are “Baker-Huang dendrimers” or “Baker-Huang PAMAM dendrimers” (see, e.g., U.S. Provisional Patent Application Ser. No. 61/251,244).

The dendrimer core structures dictate several characteristics of the molecule such as the overall shape, density and surface functionality (See, e.g., Tomalia et al., Chem. Int. Ed. Engl., 29:5305 (1990)). Spherical dendrimers can have ammonia as a trivalent initiator core or ethylenediamine (EDA) as a tetravalent initiator core. Recently described rod-shaped dendrimers (See, e.g., Yin et al., J. Am. Chem. Soc., 120:2678 (1998)) use polyethyleneimine linear cores of varying lengths; the longer the core, the longer the rod. Dendritic macromolecules are available commercially in kilogram quantities and are produced under current good manufacturing processes (GMP) for biotechnology applications.

Dendrimers may be characterized by a number of techniques including, but not limited to, electrospray-ionization mass spectroscopy, ¹³C nuclear magnetic resonance spectroscopy, ¹H nuclear magnetic resonance spectroscopy, size exclusion chromatography with multi-angle laser light scattering, ultraviolet spectrophotometry, capillary electrophoresis and gel electrophoresis. These tests assure the uniformity of the polymer population and are important for monitoring quality control of dendrimer manufacture for GMP applications and in vivo usage.

Numerous U.S. patents describe methods and compositions for producing dendrimers. Examples of some of these patents are given below in order to provide a description of some dendrimer compositions that may be useful in the present invention, however it should be understood that these are merely illustrative examples and numerous other similar dendrimer compositions could be used in the present invention.

U.S. Pat. No. 4,507,466, U.S. Pat. No. 4,558,120, U.S. Pat. No. 4,568,737, and U.S. Pat. No. 4,587,329 each describe methods of making dense star polymers with terminal densities greater than conventional star polymers. These polymers have greater/more uniform reactivity than conventional star polymers, i.e. 3rd generation dense star polymers. These patents further describe the nature of the amidoamine dendrimers and the 3-dimensional molecular diameter of the dendrimers.

U.S. Pat. No. 4,631,337 describes hydrolytically stable polymers. U.S. Pat. No. 4,694,064 describes rod-shaped dendrimers. U.S. Pat. No. 4,713,975 describes dense star polymers and their use to characterize surfaces of viruses, bacteria and proteins including enzymes. Bridged dense star polymers are described in U.S. Pat. No. 4,737,550. U.S. Pat. No. 4,857,599 and U.S. Pat. No. 4,871,779 describe dense star polymers on immobilized cores useful as ion-exchange resins, chelation resins and methods of making such polymers.

U.S. Pat. No. 5,338,532 is directed to starburst conjugates of dendrimer(s) in association with at least one unit of carried agricultural, pharmaceutical or other material. This patent describes the use of dendrimers to provide means of delivery of high concentrations of carried materials per unit polymer, controlled delivery, targeted delivery and/or multiple species such as e.g., drugs antibiotics, general and specific toxins, metal ions, radionuclides, signal generators, antibodies, interleukins, hormones, interferons, viruses, viral fragments, pesticides, and antimicrobials.

U.S. Pat. No. 6,471,968 describes a dendrimer complex comprising covalently linked first and second dendrimers, with the first dendrimer comprising a first agent and the second dendrimer comprising a second agent, wherein the first dendrimer is different from the second dendrimer, and where the first agent is different than the second agent.

Other useful dendrimer type compositions are described in U.S. Pat. No. 5,387,617, U.S. Pat. No. 5,393,797, and U.S. Pat. No. 5,393,795 in which dense star polymers are modified by capping with a hydrophobic group capable of providing a hydrophobic outer shell. U.S. Pat. No. 5,527,524 discloses the use of amino terminated dendrimers in antibody conjugates.

PAMAM dendrimers are highly branched, narrowly dispersed synthetic macromolecules with well-defined chemical structures. PAMAM dendrimers can be easily modified and conjugated with multiple functionalities such as targeting molecules, imaging agents, and drugs (Thomas et al. (2007) Poly(amidoamine) Dendrimer-based Multifunctional Nanoparticles, in Nanobiotechnology: Concepts, Methods and Perspectives, Merkin, Ed., Wiley-VCH). They are water soluble, biocompatible, and cleared from the blood through the kidneys (Peer et al. (2007) Nat. Nanotechnol. 2:751-760) which eliminates the need for biodegradability. Because of these desirable properties, PAMAM dendrimers have been widely investigated for drug delivery (Esfand et al. (2001) Drug Discov. Today 6:427-436; Patri et al. (2002) Curr. Opin. Chem. Biol. 6:466-471; Kukowska-Latallo et al. (2005) Cancer Res. 65:5317-5324; Quintana et al. (2002) Pharmaceutical Res. 19:1310-1316; Thomas et al. (2005) J. Med. Chem. 48:3729-3735), gene therapy (KukowskaLatallo et al. (1996) PNAS 93:4897-4902; Eichman et al. (2000) Pharm. Sci. Technolo. Today 3:232-245; Luo et al. (2002) Macromol. 35:3456-3462), and imaging applications (Kobayashi et al. (2003) Bioconj. Chem. 14:388-394).

The use of dendrimers as metal ion carriers is described in U.S. Pat. No. 5,560,929. U.S. Pat. No. 5,773,527 discloses non-crosslinked polybranched polymers having a comb-burst configuration and methods of making the same. U.S. Pat. No. 5,631,329 describes a process to produce polybranched polymer of high molecular weight by forming a first set of branched polymers protected from branching; grafting to a core; deprotecting first set branched polymer, then forming a second set of branched polymers protected from branching and grafting to the core having the first set of branched polymers, etc.

U.S. Pat. No. 5,902,863 describes dendrimer networks containing lipophilic organosilicone and hydrophilic polyanicloamine nanscopic domains. The networks are prepared from copolydendrimer precursors having PAMAM (hydrophilic) or polyproyleneimine interiors and organosilicon outer layers. These dendrimers have a controllable size, shape and spatial distribution. They are hydrophobic dendrimers with an organosilicon outer layer that can be used for specialty membrane, protective coating, composites containing organic organometallic or inorganic additives, skin patch delivery, absorbants, chromatography personal care products and agricultural products.

U.S. Pat. No. 5,795,582 describes the use of dendrimers as adjuvants for influenza antigen. Use of the dendrimers produces antibody titer levels with reduced antigen dose. U.S. Pat. No. 5,898,005 and U.S. Pat. No. 5,861,319 describe specific immunobinding assays for determining concentration of an analyte. U.S. Pat. No. 5,661,025 provides details of a self-assembling polynucleotide delivery system comprising dendrimer polycation to aid in delivery of nucleotides to target site. This patent provides methods of introducing a polynucleotide into a eukaryotic cell in vitro comprising contacting the cell with a composition comprising a polynucleotide and a dendrimer polycation non-covalently coupled to the polynucleotide.

In some embodiments, the modular dendrimer nanoparticle comprises a PAMAM dendrimer.

The modular dendrimer nanoparticles are not limited to having particular types of imaging agent conjugation ligands. Examples of imaging agent conjugation ligands include, but are not limited to, alkene groups, thiol groups, dieneophile groups, and diene groups. In some embodiments, the imaging agent conjugation ligands are configured for attachment with attachment ligands complexed with imaging agents.

In some embodiments, the present invention is directed towards generating modular dendrimer nanoparticles with high structural uniformity (e.g., modular dendrimer nanoparticles having precise numbers of imaging agent conjugation ligands) (e.g., modular dendrimer nanoparticles having precise numbers of imaging agents conjugated to imaging agent conjugation ligands). For example, in some embodiments, compositions of the present invention comprise ten or more modular dendrimer nanoparticles having imaging agent conjugation ligands wherein approximately 70% or higher (e.g., approximately 60%/o or higher, 63% or higher, 65%, 68%, 70/o, 70-73%, 73-75%, 75-80%, 80-81%, 81-85%, 85-90%, 90-97%, 99.99% or higher) of the modular dendrimer nanoparticles are structurally uniform (e.g., approximately 80% or more of the modular dendrimer nanoparticles have the same number of imaging agent conjugation ligands).

For example, the modular dendrimer nanoparticles are not limited to having a particular number of imaging agent conjugation ligands. In some embodiments, the modular dendrimer nanoparticles have between 1 and 128 imaging agent conjugation ligands. In some embodiments, the modular dendrimer nanoparticles have between 1 and 8 imaging agent conjugation ligands (e.g., 1 imaging agent conjugation ligand, 2 imaging agent conjugation ligands, 3 imaging agent conjugation ligands, 4 imaging agent conjugation ligands, 5 imaging agent conjugation ligands, 6 imaging agent conjugation ligands, 7 imaging agent conjugation ligands, 8 imaging agent conjugation ligands). Indeed, embodiments wherein the modular dendrimer nanoparticles have between 1 and 8 imaging agent conjugation ligands ensures that antibodies conjugated with two of such modular dendrimer nanoparticles (having conjugated imaging agents) will have between 2 and 16 imaging agents (e.g., between 1 and 8 for each modular dendrimer nanoparticle conjugated to each antibody). So as to ensure the generation of batches of modular dendrimer nanoparticles having precise numbers of imaging agent conjugation ligands, following attachment of such imaging agent conjugation ligands with dendrimer nanoparticles, isolation techniques are employed to segregate batches of dendrimer nanoparticles with precise numbers of imaging agent conjugation ligands.

The modular dendrimer nanoparticles of the present invention may be characterized for size and structural uniformity by any suitable analytical techniques. These include, but are not limited to, atomic force microscopy (AFM), electrospray-ionization mass spectroscopy, MALDI-TOF mass spectroscopy, ¹³C nuclear magnetic resonance spectroscopy, high performance liquid chromatography (HPLC), size exclusion chromatography (SEC) (equipped with multi-angle laser light scattering, dual UV and refractive index detectors), gel permeation chromatography (GPC), capillary electrophoresis and get electrophoresis. These analytical methods assure the uniformity of the dendrimer population and are important in the quality control of dendrimer production for eventual use, for example, in in vivo applications. Moreover, studies with dendrimers have shown no evidence of toxicity when administered intravenously (Roberts et al., J. Biomed. Mater. Res., 30:53 (1996) and Boume et al., J. Magnetic Resonance Imaging, 6:305 (1996)).

In certain embodiments, methods of the present invention involve conjugation of imaging agent conjugation ligands to a dendrimer to yield a population of imaging agent conjugation ligand//dendrimers, which are then subjected to high performance liquid chromatography (e.g., HPLC) (e.g., reverse-phase HPLC) to yield subpopulations of imaging agent conjugation ligand//dendrimers (e.g., subpopulations of dendrimer molecules conjugated with particular numbers of imaging agent conjugation ligands). The chromatographic traces from elution of these subpopulations are analyzed, for example, using peak fitting analysis methods to identify subpopulation (e.g., subpopulations of dendrimer molecules conjugated with particular numbers of imaging agent conjugation ligands).

For example, in some embodiments, methods of the present invention involve conjugation of at least one type of ligand to a dendrimer (e.g., conjugation of imaging agent conjugation ligands to a dendrimer) to yield a population of ligand-conjugated dendrimers, which are then subjected to reverse-phase HPLC to yield subpopulations of ligand-conjugated dendrimers. The chromatographic traces from elution of these subpopulations are analyzed, for example, using peak fitting analysis methods to identify subpopulation (e.g., subsamples, eluate fractions) wherein the structural uniformity of ligand conjugates within each subpopulation (e.g., subsample, eluate fraction) is 80% or higher (e.g., 70-73%, 73-75%, 75-80%, 80-81%, 81-85%, 85-90%, 90-97%, 99.99% or higher). Such methods are compatible with other analytical methods for structural determination or molecular analysis, such analytical methods including but not limited to nuclear magnetic resonance (NMR) (e.g., ¹H NMR), gel permeation chromatograph (GPC), mass spectrometry methods (MS) (e.g., MALDI-TOF-MS), and potentiometric titration.

Peak fitting analysis and distribution analysis are also compatible with mathematical modeling methods. Such mathematical modeling methods may include application of a two path kinetic model which allows for deviations from the Poisson distribution by varying the activation energy of the reaction a a function of n ligands on the dendrimer, e.g.,

Rₙ=A₁e^{E}^{ₐ₁}^{/(RT)}+nA₂e^{-E}^{ₐ₂}^{/(RT)} (equation 1)

In some embodiments, skewed-Poisson, Poisson, or Gaussian distribution models may be utilized to analyze dendrimer distributions.

The present invention is also directed towards products synthesized and/or prepared using methods of the present invention, e.g., by conjugation of at least one type of ligand (e.g, imaging agent conjugation ligands) to a dendrimer to yield a population of ligand-conjugated dendrimers, which are then subjected to reverse-phase HPLC to yield subpopulations of ligand-conjugated dendrimers; and analyzing the chromatographic traces from elution of these subpopulations using peak fitting analysis methods to identify subpopulation (e.g., subsamples, eluate fractions) wherein the structural uniformity of ligand conjugates within each subpopulation (e.g., subsample, eluate fraction) is 70% or higher (e.g., approximately 60% or higher, 63% or higher, 65%, 68%, 70%, 73-75%, 75-80%, 80-81%, 81-85%, 85-90%, 90-97%, 99.99% or higher) (e.g., approximately 80% or more of the modular dendrimer nanoparticles have the same number of imaging agent conjugation ligands).

Such methods are compatible with other analytical methods for structural determination or molecular analysis, such analytical methods including but not limited to nuclear magnetic resonance (NMR) (e.g., ¹H NMR), gel permeation chromatograph (GPC), mass spectrometry methods (MS) (e.g., MALDI-TOF-MS), and potentiometric titration.

The modular dendrimer nanoparticles are not limited to conjugation with a particular type of imaging agent. Examples of imaging agents include, but are not limited to, molecular dyes, fluorescein isothiocyanate (FITC), 6-TAMARA, acridine orange, and cis-parinaric acid. In some embodiments, the imaging agents are molecular dyes from the alexa fluor (Molecular Probes) family of molecular dyes. For example, examples of imaging agents include, but are not limited to, Alexa Fluor 350 (blue), Alexa Fluor 405 (violet), Alexa Fluor 430 (green), Alexa Fluor 488 (cyan-green), Alexa Fluor 500 (green), Alexa Fluor 514 (green), Alexa Fluor 532 (green), Alexa Fluor 546 (yellow), Alexa Fluor 555 (yellow-green), Alexa Fluor 568 (orange), Alexa Fluor 594 (orange-red), Alexa Fluor 610 (red), Alexa Fluor 633 (red), Alexa Fluor 647 (red), Alexa Fluor 660 (red), Alexa Fluor 680 (red), Alexa Fluor 700 (red), Alexa Fluor 750 (red), fluorescein isothiocyanate (FITC), 6-TAMARA, acridine orange, cis-parinaric acid, Hoechst 33342, Brilliant Violet™ 421, BD Horizon™ V450, Pacific Blue™, AmCyan, phycoerythrin (PE), Brilliant Violet™ 605, BD Horizon™ PE-CF594, PI, 7-AAD, allophycocyanin (APC), PE-Cy™ 5 S, PerCP, PerCP-Cy™ 5.5, PE-Cy™ 7, APC-Cy7, BD APC-H7, Texas Red, Lissamine Rhodamine B, X-Rhodamine, TRITC, Cy2, Cy3, Cy3B, Cy3.5, Cy5.5, Cy7, BODIPY-FL, FluorX™, TruRed, Red 613, NMD, Lucifer yellow, Pacific Orange, Pacific Blue, Cascade Blue, Methoxycoumarin, coumarin, hydroxycoumarin, aminocoumarin, 3-azidocoumarin, DyLight 350, DyLight 405, DyLight 488, DyLight® 550, DyLight 594, DyLight 633, DyLight® 650, DyLight 680, DyLight 755, DyLight 800, Tracy 645, Tracy 652, Atto 488, Atto 520, Atto 532, Atto Rho6G, Atto 550, Atto 565, Atto 590, Atto 594, Atto 633, Atto Rho11, Atto Rho14, Atto 647, Atto 647N, Atto 655, Atto 680, Atto 700, CF™ 350, CF™ 405S, CF™ 405M, CF™ 488A, CF™ 543, CF™ 555, CF™ 568, CF™ 594, CF™ 620R, CF™ 633, CF™ 640R, CF™ 647, CF™ 660, CF™ 660R, CF™ 680, CF™ 680R, CF™ 750, CF™ 770, and CF™ 790.

In some embodiments, the imaging agent is a mass-spec label selected from the group consisting of 139La, 141Pr, 142Nd, 143Nd, 144Nd, 145Nd, 146Nd, 147Sm, 148Nd, 149Sm, 150Nd, 151Eu, 152Sm, 153Eu, 154Sm, 156Gd, 158Gd, 159Tb, 160Gd, 162Dy, 164Dy, 165Ho, 166Er, 167Er, 168Er, 169Tm, 170Er, 171Yb, 172Yb, 174Yb, 175Lu, and 176Yb.

In some embodiments, the imaging agents are conjugated with linkage agents. Examples of such linkage agents include, but are not limited to, thiol groups, diene groups, dieneophile groups, and alkene groups. In some embodiments, the imaging agents are configured to facilitate attachment with imaging agent conjugation ligands (e.g., imaging agent conjugation ligands attached to modular dendrimer nanoparticles). For example, in some embodiments, the imaging agent linkage agent is a thiol group and the imaging agent conjugation ligand is an alkene group. In some embodiments, the imaging agent linkage agent is an alkene group and the imaging agent conjugation ligand is a thiol group. In some embodiments, the imaging agent linkage agent is a diene group and the imaging agent conjugation ligand is a dieneophile group. In some embodiments, the imaging agent linkage agent is a dieneophile group and the imaging agent conjugation ligand is a diene group.

The modular dendrimer nanoparticles are not limited to conjugation with a particular type of antibody conjugation ligand. Examples of antibody conjugation ligands include, but are not limited to, cyclooctyne groups, fluorinated cyclooctyne groups, and alkyne groups. In some embodiments, the antibody conjugation ligand is any type of ligand that facilitates conjugation with another chemical group via click chemistry. The modular dendrimer nanoparticles are not limited to having a particular number of antibody conjugation ligands. In some embodiments, the modular dendrimer nanoparticles are conjugated with one antibody conjugation ligand.

In certain embodiments, the modular dendrimer nanoparticles having precise numbers of imaging agents are conjugated with antibodies. The present invention is not limited to a particular type of antibody. In some embodiments, the antibody is a monoclonal antibody. In some embodiments, the antibody is a polyclonal antibody.

Examples of antibodies include, but are not limited to, the following antibodies shown in Table 1 and Table 2 (with type, source, and target):

TABLE 1

| Name | Type | Source | Target |
|---|---|---|---|
| 3F8 | mab | mouse | GD2 |
| 8H9 | mab | mouse | B7-H3 |
| Abagovomab | mab | mouse | CA-125 (imitation) |
| Abciximab | Fab | chimeric | CD41 (integrin |
| | | | alpha-IIb) |
| Actoxumab | mab | human | Clostridium difficile |
| Adalimumab | mab | human | TNF-a |
| Adecatumumab | mab | human | EpCAM |
| Afelimomab | F(ab′)₂ | mouse | TNF-a |
| Afutuzumab | mab | humanized | CD20 |
| Alacizumab pegol | F(ab′)₂ | humanized | VEGFR2 |
| ALD518 | ? | humanized | IL-6 |
| Alemtuzumab | mab | humanized | CD52 |
| Alirocumab | mab | human | NADP-1 |
| Altumomab | mab | mouse | CEA |
| pentate | | | |
| Amatuximab | mab | chimeric | MORAb-009 |
| Anatumomab | Fab | mouse | TAG-72 |
| mafenatox | | | |
| Anrukinzumab | mab | humanized | IL-13 |
| (= IMA-638) | | | |
| Apolizumab | mab | humanized | ITLA-DR ? |
| Arcitumomab | Fab′ | mouse | CEA |
| Aselizumab | mab | humanized | L-selectin (CD62L) |
| Atinumab | mab | human | RTN4 |
| Atlizumab | mab | humanized | IL-6 receptor |
| (= tocilizumab) | | | |
| Atorolimumab | mab | human | Rhesus factor |
| Bapineuzumab | mab | humanized | beta amyloid |
| Basiliximab | mab | chimeric | CD25 (α chain of |
| | | | IL-2 receptor) |
| Bavituximab | mab | chimeric | phosphatidylserine |
| Bectumomab | Fab′ | mouse | CD22 |
| Belimumab | mab | human | BAFF |
| Benralizumab | mab | humanized | CD125 |
| Bertilimumab | mab | human | CCL11 (eotaxin-1) |
| Besilesomab | mab | mouse | CEA-related antigen |
| Bevacizumab | mab | humanized | VEGF-A |
| Bezlotoxumab | mab | human | Clostridium difficile |
| Biciromab | Fab′ | mouse | fibrin II, beta chain |
| Bivatuzumab | mab | humanized | C7D44 v6 |
| mertansine | | | |
| Blinatumomab | BiTE | mouse | CD19 |
| Blosozumab | mab | humanized | SOST |
| Brentuximab | mab | chimeric | CD30 (TNFRSF8) |
| vedotin | | | |
| Briakinumab | mab | human | IL-12, IL-23 |
| Brodalumab | mab | human | IL-17 |
| Canakinumab | mab | human | IL-1? |
| Cantuzumab | mab | humanized | mucin CanAg |
| mertansine | | | |
| Cantuzumab | mab | humanized | MUC1 |
| ravtansine | | | |
| Caplacizumab | mab | humanized | VWF |
| Capromab | mab | mouse | prostatic carcinoma cells |
| pendetide | | | |
| Carlumab | mab | human | CNTO888 |
| Catumaxomab | 3funct | rat/mouse hybrid | EpCAM, CD3 |
| CC49 | mab | mouse | TAG-72 |
| Cedelizumab | mab | humanized | CD4 |
| Certolizumab | Fab′ | humanized | TNF-α |
| pegol | | | |
| Cetuximab | mab | chimeric | EGFR |
| Ch.14.18 | mab | chimeric | ??? |
| Citatuzumab | Fab | humanized | EpCAM |
| bogatox | | | |
| Cixutumumab | mab | human | IGF-1 receptor |
| Clazakizumab | mab | humanized | Oryctolagus cuniculus |
| Clenoliximab | mab | chimeric | CD4 |
| Clivatuzumab | mab | humanized | MUC1 |
| tetraxetan | | | |
| Conatumumab | mab | human | TRAIL-R2 |
| CR6261 | mab | human | Influenza A |
| | | | hemagglutinin |
| Crenezumab | mab | humanized | MABT5102A |
| Dacetuzumab | mab | humanized | CD40 |
| Daclizumab | mab | humanized | CD25 (α chain of |
| | | | IL-2 receptor) |
| Dalotuzumab | mab | humanized | insulin-like growth |
| | | | factor I receptor |
| Daratumumab | mab | human | CD38 (cyclic ADP |
| | | | ribose hydrolase) |
| Demcizumab | mab | humanized | DLL4 |
| Denosumab | mab | human | RANKL |
| Detumomab | mab | mouse | B-lymphoma cell |
| Dorlimomab | F(ab′)₂ | mouse | ? |
| aritox | | | |
| Drozitumab | mab | human | DR5 |
| Duligotumab | mab | human | HER3 |
| Dupilumab | mab | human | IL4 |
| Dusigitumab | mab | human | ILGF2 |
| Ecromeximab | mab | chimeric | GD3 ganglioside |
| Eculizumab | mab | humanized | C5 |
| Edobacomab | mab | mouse | endotoxin |
| Edrecolomab | mab | mouse | EpCAM |
| Efalizumab | mab | humanized | LFA-1 (CD11a) |
| Efungumab | scFv | human | Hsp90 |
| Elotuzumab | mab | humanized | SLAMF7 |
| Elsilimomab | mab | mouse | IL-6 |
| Enavatuzumab | mab | humanized | PDL192 |
| Enlimomab pegol | mab | mouse | ICAM-1 (CD54) |
| Enokizumab | mab | humanized | MEDI-528 |
| Enoticumab | mab | human | DLL4 |
| Ensituximab | mab | chimeric | NPC-1C |
| Epitumomab | mab | mouse | episialin |
| cituxetan | | | |
| Epratuzumab | mab | humanized | CD22 |
| Erlizumab | F(ab′)₂ | humanized | ITGB2 (CD18) |
| Ertumaxomab | 3funct | rat/mouse hybrid | HER2/neu, CD3 |
| Etaracizumab | mab | humanized | integrin αᵥβ₃ |
| Etrolizumab | mab | humanized | rhuMAb β₇ |
| Exbivirumab | mab | human | hepatitis B surface |
| | | | antigen |
| Fanolesomab | mab | mouse | CD15 |
| Faralimomab | mab | mouse | interferon receptor |
| Farletuzumab | mab | humanized | folate receptor 1 |
| Fasinumab | mab | human | HNGF |
| FBTA05 | 3funct | rat/mouse hybrid | CD20 |
| Felvizumab | mab | humanized | respiratory syncytial virus |
| Fezakinumab | mab | human | IL-22 |
| Ficlatuzumab | mab | humanized | SCH 900105 |
| Figitumumab | mab | human | IGF-1 receptor |
| Flanvotumab | mab | human | glycoprotein 75 |
| Fontolizumab | mab | humanized | IFN-γ |
| Foralumab | mab | human | CD3 epsilon |
| Foravirumab | mab | human | rabies virus glycoprotein |
| Fresolimumab | mab | human | TGF-β |
| Fulranumab | mab | human | NGF |
| Futuximab | mab | chimeric | EGFR |
| Galiximab | mab | chimeric | CD80 |
| Ganitumab | mab | human | IGF-I |
| Gantenerumab | mab | human | beta amyloid |
| Gavilimomab | mab | mouse | CD147 (basigin) |
| Gemtuzumab | mab | humanized | CD33 |
| ozogamicin | | | |
| Gevokizumab | mab | humanized | IL-1β |
| Girentuximab | mab | chimeric | carbonic anhydrase |
| | | | 9 (CA-IX) |
| Glembatumumab | mab | human | GPNMB |
| vedotin | | | |
| Golimumab | mab | human | TNF-α |
| Gomiliximab | mab | chimeric | CD23 (IgE receptor) |
| GS6624 | mab | ? | ? |
| Ibalizumab | mab | humanized | CD4 |
| Ibritumomab | mab | mouse | CD20 |
| tiuxetan | | | |
| Icrucumab | mab | human | VEGFR-1 |
| Igovomab | F(ab′)₂ | mouse | CA-125 |
| Imciromab | mab | mouse | cardiac myosin |
| Imgatuzumab | mab | humanized | EGFR |
| Inclacumab | mab | human | selectin P |
| Indatuximab | mab | chimeric | SDC1 |
| ravtansine | | | |
| Infliximab | mab | chimeric | TNF-α |
| Inolimomab | mab | mouse | CD25 (α chain of |
| | | | IL-2 receptor) |
| Inotuzumab | mab | humanized | CD22 |
| ozogamicin | | | |
| Intetumumab | mab | human | CD51 |
| Ipilimumab | mab | human | CD152 |
| Iratumumab | mab | human | CD30 (TNFRSF8) |
| Itolizumab | mab | humanized | CD6 |
| Ixekizumab | mab | humanized | IL-17A |
| Keliximab | mab | chimeric | CD4 |
| Labetuzumab | mab | humanized | CEA |
| Lampalizumab | mab | humanized | CFD |
| Lebrikizumab | mab | humanized | IL-13 |
| Lemalesomab | mab | mouse | NCA-90 (granulocyte |
| | | | antigen) |
| Lerdelimumab | mab | human | TGF beta 2 |
| Lexatumumab | mab | human | TRAIL-R2 |
| Libivirumab | mab | human | hepatitis B surface |
| | | | antigen |
| Ligelizumab | mab | humanized | IGHE |
| Lintuzumab | mab | humanized | CD33 |
| Lirilumab | mab | human | KIR2D |
| Lorvotuzumab | mab | humanized | CD56 |
| mertansine | | | |
| Lucatumumab | mab | human | CD40 |
| Lumiliximab | mab | chimeric | CD23 (IgE receptor) |
| Mapatumumab | mab | human | TRAIL-R1 |
| Maslimomab | ? | mouse | T-cell receptor |
| Matuzumab | mab | humanized | EGFR |
| Mavrilimuntab | mab | human | CAM-3001 |
| Mepolizumab | mab | humanized | IL-5 |
| Metelimumab | mab | human | TGF beta 1 |
| Milatuzumab | mab | humanized | CD74 |
| Minretumomab | mab | mouse | TAG-72 |
| Mitumomab | mab | mouse | GD3 ganglioside |
| Mogamulizumab | mab | humanized | CCR4 |
| Morolimumab | mab | human | Rhesus factor |
| Motavizumab | mab | humanized | respiratory syncytial |
| | | | virus |
| Moxetumomab | mab | mouse | CD22 |
| pasudotox | | | |
| Muromonab-CD3 | mab | mouse | CD3 |
| Nacolomab | Fab | mouse | C242 antigen |
| tafenatox | | | |
| Namilumab | mab | human | CSF2 |
| Naptumomab | Fab | mouse | 5T4 |
| estafenatox | | | |
| Narnatumab | mab | human | RON |
| Natalizumab | mab | humanized | integrin α₄ |
| Nebacumab | mab | human | endotoxin |
| Necitumumab | mab | human | EGFR |
| Nerelimomab | mab | mouse | TNF-α |
| Nesvacumab | mab | human | angiopoietin 2 |
| Nimotuzumab | mab | humanized | EGFR |
| Nivolumab | mab | human | IgG4 |
| Nofetumomab | Fab | mouse | ? |
| merpentan | | | |
| Ocaratuzumab | mab | humanized | CD20 |
| Ocrelizumab | mab | humanized | CD20 |
| Odulimomab | mab | mouse | LFA-1 (CD11a) |
| Ofatumumab | mab | human | CD20 |
| Olaratumab | mab | human | PDGF-R α |
| Olokizumab | mab | humanized | IL6 |
| Omalizumab | mab | humanized | IgE Fc region |
| Onartuzumab | mab | humanized | human scatter factor |
| | | | receptor kinase |
| Oportuzumab | scFv | humanized | EpCAM |
| monatox | | | |
| Oregovomab | mab | mouse | CA-125 |
| Orticumab | mab | human | oxLDL |
| Otelixizumab | mab | chimeric/humanized | CD3 |
| Oxelumab | mab | human | OX-40 |
| Ozanezumab | mab | humanized | NOGO-A |
| Ozoralizumab | mab | humanized | Lama glama |
| Pagibaximab | mab | chimeric | lipoteichoic acid |
| Palivizumab | mab | humanized | F protein of |
| | | | respiratory syncytial |
| | | | virus |
| Panitumumab | mab | human | EGFR |
| Panobacumab | mab | human | Pseudomonas |
| | | | aeruginosa |
| Parsatuzumab | mab | human | EGFL7 |
| Pascolizumab | mab | humanized | IL-4 |
| Pateclizumab | mab | humanized | LTA |
| Patritumab | mab | human | HER3 |
| Pemtumomab | ? | mouse | MUC1 |
| Perakizumab | mab | humanized | IL17A |
| Pertuzumab | mab | humanized | HER2/neu |
| Pexelizumab | scFv | humanized | C5 |
| Pidilizumab | mab | humanized | PD-1 |
| Pintumomab | mab | mouse | adenocarcinoma antigen |
| Placulumab | mab | human | human TNF |
| Ponezumab | mab | humanized | human beta-amyloid |
| Priliximab | mab | chimeric | CD4 |
| Pritumumab | mab | human | vimentin |
| PRO 140 | ? | humanized | CCR5 |
| Quilizumab | mab | humanized | IGHE |
| Racotumomab | mab | mouse | N-glycolylneuraminic |
| | | | acid |
| Radretumab | mab | human | fibronectin extra |
| | | | domain-B |
| Rafivirumab | mab | human | rabies virus glycoprotein |
| Ramucirumab | mab | human | VEGFR2 |
| Ranibizumab | Fab | humanized | VEGF-A |
| Raxibacumab | mab | human | anthrax toxin, |
| | | | protective antigen |
| Regavirumab | mab | human | cytomegalovirus |
| | | | glycoprotein B |
| Reslizumab | mab | humanized | IL-5 |
| Rilotumumab | mab | human | HGF |
| Rituximab | mab | chimeric | CD20 |
| Robatumumab | mab | human | IGF-1 receptor |
| Roledumab | mab | human | RHD |
| Romosozumab | mab | humanized | scleroscin |
| Rontalizumab | mab | humanized | IFN-α |
| Rovelizumab | mab | humanized | CD11, CD18 |
| Ruplizumab | mab | humanized | CD154 (CD40L) |
| Samalizumab | mab | humanized | CD200 |
| Sarilumab | mab | human | IL6 |
| Satumomab | mab | mouse | TAG-72 |
| pendetide | | | |
| Secukinumab | mab | human | IL-17A |
| Sevirumab | ? | human | cytomegalovirus |
| Sibrotuzumab | mab | humanized | FAP |
| Sifalimumab | mab | humanized | IFN-α |
| Siltuximab | mab | chimeric | IL-6 |
| Simtuzumab | mab | humanized | LOXL2 |
| Siplizumab | mab | humanized | CD2 |
| Sirukumab | mab | human | IL-6 |
| Solanezumab | mab | humanized | beta amyloid |
| Solitomab | mab | mouse | EPCAM |
| Sonepcizumab | ? | humanized | sphingosine-1-phosphate |
| Sontuzumab | mab | humanized | episialin |
| Stamulumab | mab | human | myostatin |
| Sulesomab | Fab′ | mouse | NCA-90 (granulocyte |
| | | | antigen) |
| Suvizumab | mab | humanized | HIV-1 |
| Tabalumab | mab | human | BAFF |
| Tacatuzumab | mab | humanized | alpha-fetoprotein |
| tetraxetan | | | |
| Tadocizumab | Fab | humanized | integrin α_{IIb}β₃ |
| Talizumab | mab | humanized | IgE |
| Tanezumab | mab | humanized | NGF |
| Taplitumomab | mab | mouse | CD19 |
| paptox | | | |
| Tefibazumab | mab | humanized | clumping factor A |
| Telimomab aritox | Fab | mouse | ? |
| Tenatumomab | mab | mouse | tenascin C |
| Teneliximab | mab | chimeric | CD40 |
| Teplizumab | mab | humanized | CD3 |
| Teprotumumab | mab | human | CD221 |
| TGN1412 | ? | humanized | CD28 |
| Ticilimumab (= | mab | human | CTLA-4 |
| tremelimumab) | | | |
| Tigatuzumab | mab | humanized | TRAIL-R2 |
| Tildrakizumab | mab | humanized | IL23 |
| TNX-650 | ? | humanized | IL-13 |
| Tocilizumab | mab | humanized | IL-6 receptor |
| (= atlizumab) | | | |
| Toralizumab | mab | humanized | CD154 (CD40L) |
| Tositumomab | ? | mouse | CD20 |
| Tralokinumab | mab | human | IL-13 |
| Trastuzumab | mab | humanized | HER2/neu |
| TRBS07 | 3funct | ? | GD2 |
| Tregalizumab | mab | humanized | CD4 |
| Tremelimumab | mab | human | CTLA-4 |
| Tucotuzumab | mab | humanized | EpCAM |
| celmoleukin | | | |
| Tuvirumab | ? | human | hepatitis B virus |
| Ublituximab | mab | chimeric | MS4A1 |
| Urelumab⁰ | mab | human | 4-1BB |
| Urtoxazumab | mab | humanized | Escherichia coli |
| Ustekinumab | mab | human | IL-12, IL-23 |
| Vapaliximab | mab | chimeric | AOC3 (VAP-1) |
| Vatelizumab | mab | humanized | ITGA2 |
| Vedolizumab | mab | humanized | integrin α₄β₇ |
| Veltuzumab | mab | humanized | CD20 |
| Vepalimomab | mab | mouse | AOC3 (VAP-1) |
| Vesencumab | mab | human | NRP1 |
| Visilizumab | mab | humanized | CD3 |
| Volociximab | mab | chimeric | integrin α₅β₁ |
| Vorsetuzumab | mab | humanized | cancer |
| mafodotin | | | |
| Votumumab | mab | human | tumor antigen |
| | | | CTAA16.88 |
| Zalutumumab | mab | human | EGFR |
| Zanolimumab | mab | human | CD4 |
| Zatuximab | mab | chimeric | HER1 |
| Ziralimumab | mab | human | CD147 (basigin) |
| Zolimomab aritox | mab | mouse | CD5 |

(mab: whole monoclonal antibody) (Fab: fragment, antigen-binding (one arm) (F(ab′)₂: fragment, antigen-binding, including hinge region (both arms)) (Fab′: fragment, antigen-binding, including hinge region (one arm)) (scFv: single-chain variable fragment) (di-scFv: dimeric single-chain variable fragment) (sdAb: single-domain antibody) (3funct: trifunctional antibody) (BiTE: bi-specific T-cell engager)

TABLE 2

| Name | Type | Source | Target |
|---|---|---|---|
| Mouse | mab | mouse | Bovine IgG |
| anti- | | | |
| Bovine | | | |
| IgG, LC | | | |
| Mouse Bv | | | |
| Secondary | | | |
| Antibody | | | |
| (IVA285-1) | | | |
| Mouse anti-Bovine | mab | Mouse | |
| IgM Secondary | | | |
| Antibody (G9) | | | |
| Rabbit anti-Bovine | Mab | Rabbit | |
| IgG Secondary | | | |
| Antibody | | | |
| Mouse anti-Bovine Ig | Mab | Mouse | |
| Secondary Antibody | | | |
| (BIG10-101.6) | | | |
| Mouse anti-Bovine Ig | mab | Mouse | |
| Secondary Antibody | | | |
| (BIG10-123.1) | | | |
| Mouse anti-Bovine Ig | mab | mouse | |
| Secondary Antibody | | | |
| (BIG10-137.4) | | | |
| Rabbit anti-Bovine | mab | Rabbit | |
| IgG/IgM/IgA | | | |
| Secondary Antibody | | | |
| Mouse anti-Canine Ig | | Mouse | |
| Secondary Antibody | | | |
| (DIG11-124.1) | | | |
| Mouse anti-Canine Ig | | Mouse | |
| Secondary Antibody | | | |
| (DIG12-223.3) | | | |
| Rabbit anti-Chicken | Polyclonal | Rabbit | Chicken IgY |
| IgY (H + L) Secondary | | | (H + L) |
| Antibody | | | |
| Goat anti-Chicken | Polyclonal | Goat | Chicken IgY |
| IgY Secondary | | | |
| Antibody | | | |
| Mouse anti-Chicken | mab | Mouse | Chicken Ig |
| Ig Secondary | | | |
| Antibody (CIG10- | | | |
| 196.101) | | | |
| Goat anti-Chicken | polyclonal | Goat | Chicken IgG |
| IgG, H&L chains | | | (H + L) |
| Secondary Antibody | | | |
| Mouse anti-Chicken | Mab | Mouse | Chicken IgG |
| IgG Secondary | | | |
| Antibody (409-3.1) | | | |
| Mouse anti-Chicken | mab | Mouse | Chicken |
| IgG/IgM/IgA | | | IgG/IgM/IgA |
| Secondary Antibody | | | |
| (408-6.1) | | | |
| Mouse anti-Chicken | mab | Mouse | Chicken IgM |
| IgM Secondary | | | |
| Antibody (408-5.1) | | | |
| Goat anti-Chicken | Polyclonal | Goat | Chicken IgY |
| IgY Secondary | | | |
| Antibody | | | |
| Donkey anti-Chicken | Polyclonal | Donkey | Chicken IgY |
| IgY Secondary | | | |
| Antibody | | | |
| Goat anti-Chicken | Polyclonal | Goat | Chicken IgY Fab |
| IgY, Fab Secondary | | | |
| Antibody | | | |
| Bovine anti-Chicken | Polyclonal | Bovine | Chicken IgY |
| IgY Secondary | | | |
| Antibody | | | |
| Goat anti-Donkey | Polyclonal | Goat | Donkey IgG |
| IgG Secondary | | | |
| Antibody | | | |
| Rabbit anti-Donkey | Polyclonal | Rabbit | Donkey IgG, H&L |
| IgG, H&L chains | | | chains |
| Secondary Antibody | | | |
| Mouse anti-Feline Ig | Mab | Mouse | Feline Ig |
| Secondary Antibody | | | |
| (FIG10-102 .8) | | | |
| Mouse anti-Feline Ig | mab | Mouse | Feline Ig |
| Secondary Antibody | | | |
| (FIG10-118.1) | | | |
| Mouse anti-Feline Ig | mab | Mouse | Feline Ig |
| Secondary Antibody | | | |
| (FIG11-207.2) | | | |
| Goat anti-Feline IgG | Polyclonal | Goat | Feline Ig |
| Secondary Antibody | | | |
| Rabbit anti-Goat IgG | Polyclonal | Rabbit | Goat IgG, H&L |
| (H + L) Secondary | | | chains |
| Antibody | | | |
| Mouse anti-Goat IgG | Polyclonal | Mouse | Goat IgG, H&L |
| (H + L) Cross | | | chains |
| Adsorbed Secondary | | | |
| Antibody | | | |
| F(ab′)2-Rabbit anti- | Poly | Rabbit | Goat IgG, H&L |
| Goat IgG (H + L) | | | chains |
| Cross Adsorbed | | | |
| Secondary Antibody | | | |
| Rabbit anti-Goat IgG | Poly | Rabbit | Goat IgG, Fc |
| (Fc) Secondary | | | |
| Antibody | | | |
| Donkey anti-Goat | Poly | Donkey | Goat IgG |
| IgG Secondary | | | |
| Antibody | | | |
| Mouse anti-Goat Ig | Mab | Mouse | Goat Ig |
| Secondary Antibody | | | |
| (GIG10-115.25) | | | |
| Mouse anti-Guinea | mab | Mouse | Guinea pig IgG |
| pig IgG Secondary | | | |
| Antibody (MsGp3) | | | |
| Rabbit anti-Guinea | Poly | Rabbit | Guinea pig IgG |
| Pig IgG Secondary | | | |
| Antibody | | | |
| Goat anti-Guinea Pig | Poly | Goat | Guinea pig IgG |
| IgG Secondary | | | |
| Antibody | | | |
| Goat anti-Hamster | poly | Goat | Hamster IgG, H&L |
| IgG (H + L) | | | chains |
| Secondary Antibody | | | |
| Rabbit anti-Hamster | Poly | Rabbit | Hamster IgG, H&L |
| IgG, H&L chains | | | chains |
| Secondary Antibody | | | |
| Goat anti-Human | Poly | Goat | Human IgG Gamma |
| Gamma Chain | | | Chain |
| Secondary Antibody | | | |
| Goat anti-Human IgG | Poly | Goat | Human IgG, H&L |
| (H + L) Cross | | | Chains |
| Adsorbed Secondary | | | |
| Antibody | | | |
| Goat anti-Human | Poly | Goat | Human IgG |
| IgG F(ab′)2 | | | F(ab′)2 |
| Secondary Antibody | | | |
| Goat anti-Human | Poly | Goat | Human IgM |
| IgM Secondary | | | |
| Antibody | | | |
| Goat anti-Human IgG | poly | Goat | Human IgG |
| Cross Adsorbed | | | |
| Secondary Antibody | | | |
| Goat anti-Human IgA + | Poly | Goat | Human IgA, IgG, |
| + IgG + IgM (H + L) | | | IgM, H&L chains |
| Secondary Antibody | | | |
| Goat anti-Human | Poly | Goat | Human IgG Kappa |
| Kappa Chain | | | Chain |
| Secondary Antibody | | | |
| Goat anti-Human IgG | Poly | Goat | Human IgG |
| Cross Adsorbed | | | |
| Secondary Antibody | | | |
| Mouse anti-Human | Poly | Mouse | Human IgG H&L |
| IgG (H + L) Cross | | | chains |
| Adsorbed Secondary | | | |
| Antibody | | | |
| Goat anti-Human | Poly | Goat | Human IgA |
| IgA (a) Secondary | | | |
| Antibody | | | |
| Rabbit anti-Human | Poly | Rabbit | Human IgG Fc |
| IgG (Fc) Secondary | | | |
| Antibody | | | |
| Rabbit anti-Human | Poly | Rabbit | Human IgG H&L |
| IgG (H + L) | | | chains |
| Secondary Antibody | | | |
| F(ab′)2-Goat anti- | Poly | Goat | Human IgG, Fc- |
| Human IgG (FC- | | | gamma |
| gamma) Secondary | | | |
| Antibody | | | |
| F(ab′)2-Goat anti- | Poly | Goat | Human IgG H&L |
| Human IgG (H + L) | | | chains |
| Secondary Antibody | | | |
| Duck anti-Human | Poly | Duck | Human IgG |
| IgG Secondary | | | |
| Antibody | | | |
| Mouse anti-Human | Mono | Mouse | Human IgM |
| IgM Secondary | | | |
| Antibody (13A11) | | | |
| Mouse anti-Human | Mono | Mouse | Human IgA |
| IgA Secondary | | | |
| Antibody (47C12) | | | |
| Mouse anti-IgG (Fc) | Mono | Mouse | Human IgG Fc |
| Secondary Antibody | | | |
| (EM-07) | | | |
| Mouse anti-Human | Mono | Mouse | Human IgG3, |
| IgG3, hinge region | | | hinge |
| Secondary Antibody | | | region |
| (HP6050) | | | |
| Mouse anti-Human | Mono | Mouse | Human IgG1 |
| IgG1 Secondary | | | |
| Antibody (2C11) | | | |
| Mouse anti-Human | Mono | Mouse | Human IgG2 |
| IgG2, Fab Secondary | | | |
| Antibody (HP6014) | | | |
| Mouse anti-Human Ig | Mono | Mouse | Human Ig Light |
| Light chain | | | Chain |
| Secondary Antibody | | | |
| (7A9) | | | |
| Mouse anti-Human | Mono | Mouse | Human Ig |
| Ig, kappa LC | | | Kappa LC |
| Secondary Antibody | | | |
| (2B7) | | | |
| Mouse anti-Human | Mono | Mouse | Human IgE |
| IgE Secondary | | | |
| Antibody (BL-E9) | | | |
| Mouse anti-Human | Mono | Mouse | Human IgG4 |
| IgG4 Secondary | | | |
| Antibody (BL-G4/1) | | | |
| Mouse anti-Human | Mono | Mouse | Human IgA HC |
| IgA, HC Secondary | | | |
| Antibody (Mc24- | | | |
| 2E11) | | | |
| Mouse anti-Human | Mono | Mouse | Human and |
| IgG4 Secondary | | | Chimpanzee IgG4 |
| Antibody (HP6025) | | | |
| Mouse anti-Human | Mono | Mouse | Human IgG1 Fc |
| IgG1, Fc Secondary | | | |
| Antibody (8c/6-39 | | | |
| (HP6091)) | | | |
| Mouse anti-Human | Mono | Mouse | Human IgG Fc |
| IgG, Fc Secondary | | | |
| Antibody (8A4) | | | |
| Mouse anti-Human | Mono | Mouse | Human IgE |
| IgE Secondary | | | |
| Antibody (E411 | | | |
| (5H2)) | | | |
| Mouse anti-Human | Mono | Mouse | Human IgG1 Fc |
| IgG-1, Fc Secondary | | | |
| Antibody (2C11) | | | |
| Mouse anti-Human | Mono | Mouse | Human IgG2 |
| IgG2 Secondary | | | |
| Antibody (3C7) | | | |
| Mouse anti-Human | Mono | Mouse | Human IgG3 |
| IgG3 Secondary | | | |
| Antibody (5G12) | | | |
| Mouse anti-Human | Mono | Mouse | Human IgG3 |
| IgG CH3 domain | | | domain |
| Secondary Antibody | | | |
| (A57H) | | | |
| Mouse anti-Human | Mono | Mouse | Human IgG1 Fc |
| IgG1, Fc Secondary | | | |
| Antibody (8c/6-39) | | | |
| Rat anti-Human | Mono | Rat | Human IgG2a |
| IgG2a Secondary | | | |
| Antibody (LO-DNP- | | | |
| 16) | | | |
| Mouse anti-Human | Mono | Mouse | Human IgD |
| IgD Secondary | | | |
| Antibody (IgD26) | | | |
| Mouse anti-Human | Mono | Mouse | Human IgA alpha- |
| IgA (alpha-Heavy | | | heavy chain |
| Chain) Secondary | | | |
| Antibody (GA01) | | | |
| Mouse anti-human | Mono | Mouse | Human IgG |
| IgG Secondary | | | |
| Antibody | | | |
| (4D2D9G8) | | | |
| Goat anti-Human | Poly | Goat | Human IgG Fc |
| IgG, Fc Secondary | | | |
| Antibody | | | |
| Rabbit anti-Human | Poly | Rabbit | Human IgA alpha- |
| IgA (alpha-Heavy | | | Heavy Chain |
| Chain) Secondary | | | |
| Antibody | | | |
| Rabbit anti-Human | Poly | Rabbit | Human IgE |
| IgE (epsilon-Heavy | | | epsilon- |
| Chain) Secondary | | | Heavy Chain |
| Antibody | | | |
| Rabbit anti-Human | Poly | Rabbit | Human IgD delta- |
| IgD (delta-Heavy | | | Heavy Chain |
| Chain) Secondary | | | |
| Antibody | | | |
| Sheep anti-Human | Poly | Sheep | Human IgA |
| IgA Secondary | | | |
| Antibody | | | |
| Chicken anti-Human | Poly | Chicken | Human IgE |
| IgE Secondary | | | |
| Antibody | | | |
| Chicken anti-Human | Poly | Chicken | Human IgA |
| IgA Secondary | | | |
| Antibody | | | |
| Mouse anti-Human Ig | Mono | Mouse | Haman Ig |
| Secondary Antibody | | | |
| (HIG10-101.1.19) | | | |
| Mouse anti-Human | Mono | Mouse | Human IgG |
| IgG, lambda LC | | | lambda-LC |
| Secondary Antibody | | | |
| (ICO-106) | | | |
| Mouse anti-Human | Mono | Mouse | Human Ig |
| Ig, kappa LC | | | kappa LC |
| Secondary Antibody | | | |
| (MEM-09) | | | |
| Mouse anti-Human | N ono | Mouse | Human Ig lambda |
| Ig, lambda LC | LC | | |
| Secondary Antibody | | | |
| (Rs4) | | | |
| Mouse anti-Human | Mono | Mouse | Human IgG Fab′2 |
| IgG, Fab′2 Secondary | | | |
| Antibody (4A11) | | | |
| Mouse anti-Human | Mono | Mouse | Human IgM |
| IgG, Fab′2 Secondary | | | |
| Antibody (4A11) | | | |
| Mouse anti-Human | Mono | Mouse | Human IgM |
| IgM Secondary | | | |
| Antibody (MA2) | | | |
| Mouse anti-Human | Mono | Mouse | Human IgA |
| IgA Secondary | | | |
| Antibody (AD3) | | | |
| Mouse anti-Human | Mono | Mouse | Human IgE |
| IgE Secondary | | | |
| Antibody (BE5) | | | |
| Mouse anti-Human | Mono | Mouse | Human IgE |
| IgE Secondary | | | |
| Antibody (4G7) | | | |
| Mouse anti-Human | Mono | Mouse | Human IgE |
| IgE Secondary | | | |
| Antibody (4H10) | | | |
| Mouse anti-Human | Mono | Mouse | Human IgM |
| IgM Secondary | | | |
| Antibody (2A6) | | | |
| Mouse anti-Human | Mono | Mouse | Human IgM |
| IgM Secondary | | | |
| Antibody (MA2) | | | |
| Mouse anti-Human | Mono | Mouse | Human IgM |
| IgM Secondary | | | |
| Antibody (ICL-931) | | | |
| Mouse anti-Human | Mono | Mouse | Human IgG |
| IgG Secondary | | | |
| Antibody (EFE-565) | | | |
| Mouse anti-Human | Mono | Mouse | Human IgE |
| IgE Secondary | | | |
| Antibody (MH25/1) | | | |
| Mouse anti-Human | Mono | Mouse | Human IgM |
| IgM Secondary | | | |
| Antibody (MH15-1) | | | |
| Goat anti-Human IgG | Poly | Goat | Human IgG |
| Secondary Antibody | | | |
| Goat anti-Human | Poly | Goat | Human IgG/ |
| IgG/IgM/IgA | | | IgM/IgA |
| Secondary Antibody | | | |
| Rabbit anti-Human | Poly | Rabbit | Human IgM |
| IgM Secondary | | | |
| Antibody | | | |
| Rabbit anti-Human | Poly | Rabbit | Human IgG Fe |
| IgG, Fc gamma | | | gamma |
| Secondary Antibody | | | |
| Chicken anti-Human | Poly | Chicken | Human IgG |
| IgG Secondary | | | |
| Antibody | | | |
| Chicken anti-Human | Poly | Chicken | Human IgG Fc |
| IgG, Fc Secondary | | | |
| Antibody | | | |
| Chicken anti-Human | Poly | Chicken | Human IgM |
| IgM Secondary | | | |
| Antibody | | | |
| Mouse anti-Human | Mono | Mouse | Human IgA |
| IgA Sccondary | | | |
| Antibody (KT13) | | | |
| Mouse anti-Human | Mono | Mouse | Human IgM |
| IgM Secondary | | | |
| Antibody (KTI6) | | | |
| Bovine anti-Human | Poly | Bovine | Human IgG |
| IgG Secondary | | | |
| Antibody | | | |
| Rabbit anti-Equine | Poly | Rabbit | Horse IgG/ |
| IgG/IgM/IgA | | | IgM/IgA |
| Secondary Antibody | | | |
| Mouse anti-Monkey | Mono | Mouse | Monkey IgG |
| IgG Secondary | | | |
| Antibody (5C12.D4) | | | |
| Mouse anti-Monkey | Mono | Mouse | Monkey IgG |
| IgG Secondary | | | |
| Antibody (4D8.B10) | | | |
| Goat anti-Monkey | Poly | Goat | Monkey IgG H&L, |
| IgG, H&L chains | | | chains |
| Secondary Antibody | | | |
| Goat anti-Mouse IgG (H + L) | Poly | Goat | Mouse IgG H&L |
| Secondary Antibody | | | chains |
| Goat anti-Mouse IgG (H + L) | Poly | Goat | Mouse IgG H&L |
| Cross Adsorbed Secondary | | | |
| Antibody | | | |
| Goat anti-Mouse IgG | Poly | Goat | Mouse IgG F(ab′)2 |
| F(ab′)2 Secondary Antibody | | | |
| Goat anti-Mouse IgG (Fc) | Poly | Goat | Mouse IgG Fc |
| Secondary Antibody | | | |
| Goat anti-Mouse IgA Cross | Poly | Goat | Mouse IgA |
| Adsorbed Secondary | | | |
| Antibody | | | |
| Goat anti-Mouse IgG (Fc) | Poly | Goat | Mouse IgG Fc |
| Cross Adsorbed Secondary | | | |
| Antibody | | | |
| Goat anti-Mouse IgM | Poly | Goat | Mouse IgM |
| Secondary Antibody | | | |
| F(ab′)2-Goat anti-Mouse | Poly | F(ab′)2- | Mouse IgM |
| IgM (μ) Secondary | | Goat | |
| Antibody | | | |
| Horse anti-Mouse IgG | Poly | Horse | Mouse IgG H&L |
| (H + L) Secondary Antibody | | | chains |
| Goat anti-Mouse IgG + IgM | Poly | Goat | Mouse IgG, IgM |
| (H + L) Secondary Antibody | | | H&L chains |
| F(ab′)2-Goat anti-Mouse | Poly | F(ab′)2- | Mouse IgG H&L |
| IgG (H + L) Cross Adsorbed | | Goat | chains |
| Secondary Antibody | | | |
| F(ab′)2-Goat anti-Mouse | Poly | F(ab′)2- | Mouse IgM |
| IgM Cross Adsorbed | | Goat | |
| Secondary Antibody | | | |
| Rabbit anti-Mouse IgG | Poly | Rabbit | Mouse IgG H&L |
| (H + L) Secondary Antibody | | | chains |
| Rabbit anti-Mouse IgG | Poly | Rabbit | Mouse IgG H&L |
| (H + L) Cross Adsorbed | | | chains |
| Secondary Antibody | | | |
| Rabbit anti-Mouse IgG | Poly | Rabbit | Mouse IgG |
| F(ab′)2 Secondary Antibody | | | F(ab′)2 |
| Rabbit anti-Mouse IgG (Fc) | Poly | Rabbit | Mouse IgG Fc |
| Secondary Antibody | | | |
| Rabbit anti-Mouse IgM | Poly | Rabbit | Mouse IgM |
| Secondary Antibody | | | |
| Rabbit anti-Mouse IgG + | Poly | Rabbit | Mouse IgG, IgM |
| IgM (H + L) Secondary | | | H&L chains |
| Antibody | | | |
| Goat anti-Mouse | Poly | Goat | Mouse IgG1 |
| IgG1 + 2a + 2b + 3 Cross | | | IgG2a, IgG2b, |
| Adsorbed Secondary | | | IgG3 |
| Antibody | | | |
| Goat anti-Mouse IgG1 | Poly | Goat | Mouse IgG1 |
| Cross Adsorbed Secondary | | | |
| Antibody | | | |
| Goat anti-Mouse IgG2a | Poly | Goat | Mouse IgG2a |
| Cross Adsorbed Secondary | | | |
| Antibody | | | |
| Rat anti-Mouse IgG, LC | Mono | Rat | Mouse IgG, Lc |
| Secondary Antibody (LO- | | | |
| MK-1) | | | |
| Rat anti-Mouse IgG, HC | Mono | Rat | Mouse IgG, HC |
| Secondary Antibody | | | |
| (Cocktail) | | | |
| Goat anti-Mouse IgG | Poly | Goat | Mouse IgG |
| Secondary Antibody | | | |
| Goat anti-Mouse IgG2a | Poly | Goat | Mouse IgG2a |
| Secondary Antibody | | | |
| Goat anti-Mouse IgG2c | Poly | Goat | Mouse IgG2c |
| Secondary Antibody | | | |
| Goat anti-Mouse IgE | Poly | Goat | Mouse IgE |
| Secondary Antibody | | | |
| Goat anti-Mouse IgA | Poly | Goat | Mouse IgA |
| Secondary Antibody | | | |
| Rabbit anti-Mouse IgA | Poly | Rabbit | Rabbit IgA |
| Secondary Antibody | | | |
| Goat anti-Mouse | Poly | Goat | Mouse IgG, IgM |
| IgG/IgM/IgA, H&L chains | | | IgA H&L chains |
| Secondary Antibody | | | |
| Goat anti-Mouse IgG, Fab | Poly | Goat | Mouse IgG Fab |
| Secondary Antibody | | | |
| Rat anti-Mouse IgG3, | Mono | Rat | Mouse IgG3 |
| Heavy chain Secondary | | | heavy |
| Antibody (LO-MG3-13) | | | chain |
| Rat anti-Mouse IgG2a | Mono | Rat | Mouse IgG2a |
| Secondary Antibody (LO- | | | |
| MG2a-2) | | | |
| Rat anti-Mouse Ig, kappa | Mono | Rat | Mouse Ig kappa |
| LC Secondary Antibody | | | light chain |
| (OX-20) | | | |
| Rat anti-Mouse IgA | Mono | Rat | Mouse IgA |
| Secondary Antibody (LO- | | | |
| MA-7) | | | |
| Rat anti-Mouse IgE | Mono | Rat | Mouse IgE |
| Secondary Antibody (LO- | | | |
| ME-3) | | | |
| Rat anti-Mouse IgG | Mono | Rat | Mouse IgG |
| Secondary Antibody (LO- | | | |
| MG-7) | | | |
| Rat anti-Mouse IgG1 | Mono | Rat | Mouse IgG1 |
| Secondary Antibody (LO- | | | |
| MG1-2) | | | |
| Rat anti-Mouse IgG2a | Mono | Rat | Mouse IgG2a |
| Secondary Antibody (LO- | | | |
| MG2a-2) | | | |
| Rat anti-Mouse IgG2b | Mono | Rat | Mouse IgG2b |
| Secondary Antibody (LO- | | | |
| MG2b-2 | | | |
| Rat anti-Mouse IgG3 | Mono | Rat | Mouse IgG3 |
| Secondary Antibody (LO- | | | |
| MG3-7) | | | |
| Rat anti-Mouse IgM | Mono | Rat | Mouse IgM |
| Secondary Antibody (LO- | | | |
| MM-9) | | | |
| Chicken anti-Mouse IgG | Mono | Chicken | Mouse IgG |
| Secondary Antibody | | | |
| Chicken anti-Mouse IgG, | Poly | Chicken | Mouse IgG Fab |
| Fab Secondary Antibody | | | |
| Chicken anti-Mouse IgG, Fc | Poly | Chicken | Mouse IgG Fc |
| Secondary Antibody | | | |
| Bovine anti-Mouse IgG | Poly | Bovine | Mouse IgG |
| Secondary Antibody | | | |
| Donkey anti-Mouse IgG | Poly | Donkey | Mouse IgG |
| Secondary Antibody | | | |
| Goat anti-Rabbit IgG (H + L) | Poly | Goat | Rabbit IgG H&L |
| Secondary Antibody | | | chains |
| Goat anti-Rabbit IgG (H + L) | Poly | Goat | Rabbit IgG H&L |
| Cross Adsorbed Secondary | | | chains |
| Antibody | | | |
| Mouse anti-Rabbit IgG | Poly | Mouse | Rabbit IgG H&L |
| (H + L) Cross Adsorbed | | | chains |
| Secondary Antibody | | | |
| Goat anti-Rabbit IgG (Fc) | Poly | Goat | Rabbit IgG Fc |
| Secondary Antibody | | | |
| Goat anti-Rabbit IgG | Poly | Goat | Rabbit IgG |
| F(ab′)2 Secondary Antibody | | | F(ab′)2 |
| Donkey anti-Rabbit IgG | Poly | Donkey | Rabbit IgG H&L |
| (H + L) Cross Adsorbed | | | chains |
| Secondary Antibody | | | |
| F(ab′)2-Goat anti-Rabbit | Poly | F(ab′)2- | Rabbit IgG H&L |
| IgG (H + L) Cross Adsorbed | | Goat | chains |
| Secondary Antibody | | | |
| Goat anti-Rabbit IgM | Poly | Goat | Rabbit IgM |
| Secondary Antibody | | | |
| Rabbit anti-Rat IgG (H + L) | Poly | Rabbit | Rat IgG H&L |
| Secondary Antibody | | | chains |
| Rabbit anti-Rat IgG (H + L) | Poly | Rabbit | Rat IgG H&L |
| Cross Adsorbed Secondary | | | chains |
| Antibody | | | |
| Goat anti-Rat IgG (H + L) | Poly | Goat | Rat IgG H&L |
| Secondary Antibody | | | chains |
| Goat anti-Rat IgG (Fc) | Poly | Goat | Rat IgG Fc |
| Secondary Antibody | | | |
| Mouse anti-Rat IgG1 | Mono | Mouse | Rat IgG1 |
| Secondary Antibody | | | |
| (W3/25) | | | |
| Mouse anti-Rat IgG1 | Mono | Mouse | Rat IgG1 |
| Secondary Antibody | | | |
| (W3/25) | | | |
| Mouse anti-Rat IgE | Mono | Mouse | Rat IgE |
| Secondary Antibody | | | |
| (MARE-1) | | | |
| Chicken anti-Rat IgG | Poly | Chicken | Rat IgG |
| Secondary Antibody | | | |
| Goat anti-Rat IgG | Poly | Goat | Rat IgG |
| Secondary Antibody | | | |
| Sheep anti-Rat IgG | Poly | Sheep | Rat IgG |
| Secondary Antibody | | | |
| Goat anti-Rat IgE | Poly | Goat | Rat IgE |
| Secondary Antibody | | | |
| Sheep anti-Rat IgE | Poly | Sheep | Rat IgE |
| Secondary Antibody | | | |
| Rabbit anti-Rat IgG | Poly | Rabbit | Rat IgG |
| Secondary Antibody | | | |
| Goat anti-Rat IgG2a | Poly | Goat | Rat IgG2a |
| Secondary Antibody | | | |
| Mouse anti-Rat Ig | Mono | Mouse | Rat Ig |
| Secondary Antibody | | | |
| (RATIG12-306.21) | | | |
| Mouse anti-Rat IgG1 | Mono | Mouse | Rat IgG1 |
| Secondary Antibody | | | |
| (MARG1-2) | | | |
| Mouse anti-Rat IgG2c | Mono | Mouse | Rat IgG2c |
| Secondary Antibody | | | |
| (MARG2c-5) | | | |
| Rabbit anti-Rat | Poly | Rabbit | Rat IgG/ |
| IgG/IgM/IgA Secondary | | | IgM/IgA |
| Antibody | | | |
| Mouse anti-Rat Ig | Mono | Mouse | Rat Ig |
| Secondary Antibody (OX- | | | |
| 12) | | | |

Definition of Terms: Poly—polyclonal; Mono—monoclonal; H—heavy; L—light

In some embodiments, the antibodies recognize, for example, tumor-specific epitopes (e.g., TAG-72 (See, e.g., Kjeldsen et al., Cancer Res. 48:2214-2220 (1988); U.S. Pat. Nos. 5,892,020; 5,892,019; and 5,512,443); human carcinoma antigen (See, e.g., U.S. Pat. Nos. 5,693,763; 5,545,530; and 5,808,005); TP1 and TP3 antigens from osteocarcinoma cells (See, e.g., U.S. Pat. No. 5,855,866); Thomsen-Friedenreich (TF) antigen from adenocarcinoma cells (See, e.g., U.S. Pat. No. 5,110,911); “KC-4 antigen” from human prostrate adenocarcinoma (See, e.g., U.S. Pat. Nos. 4,708,930 and 4,743,543); a human colorectal cancer antigen (See, e.g., U.S. Pat. No. 4,921,789); CA125 antigen from cystadenocarcinoma (See, e.g., U.S. Pat. No. 4,921,790); DF3 antigen from human breast carcinoma (See, e.g., U.S. Pat. Nos. 4,963,484 and 5,053,489); a human breast tumor antigen (See. e.g., U.S. Pat. No. 4,939,240); p97 antigen of human melanoma (See, e.g., U.S. Pat. No. 4,918,164); carcinoma or orosomucoid-related antigen (CORA)(See, e.g., U.S. Pat. No. 4,914,021); a human pulmonary carcinoma antigen that reacts with human squamous cell lung carcinoma but not with human small cell lung carcinoma (See. e.g., U.S. Pat. No. 4,892,935); T and Tn haptens in glycoproteins of human breast carcinoma (See, e.g., Springer et al., Carbohydr. Res. 178:271-292 (1988)), MSA breast carcinoma glycoprotein termed (See. e.g., Tjandra et al., Br. J. Surg. 75:811-817 (1988)); MFGM breast carcinoma antigen (See, e.g., Ishida et al., Tumor Biol. 10:12-22 (1989)); DU-PAN-2 pancreatic carcinoma antigen (See, e.g., Lan et al., Cancer Res. 45:305-310 (1985)); CA125 ovarian carcinoma antigen (See, e.g., Hanisch et al., Carbohydr. Res. 178:29-47 (1988)); YH206 lung carcinoma antigen (See, e.g., Hinoda et al., (1988) Cancer J. 42:653-658 (1988)).

Various procedures known in the art are used for the production of polyclonal antibodies. For the production of antibody, various host animals can be immunized by injection with the peptide corresponding to the desired epitope including but not limited to rabbits, mice, rats, sheep, goats, etc. In a preferred embodiment, the peptide is conjugated to an immunogenic carrier (e.g., diphtheria toxoid, bovine serum albumin (BSA), or keyhole limpet hemocyanin (KLH)). Various adjuvants are used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (Bacille Calmette-Guerin) and Corynebacterium parvum.

For preparation of monoclonal antibodies, any technique that provides for the production of antibody molecules by continuous cell lines in culture may be used (See e.g., Harlow and Lane. Antibodies: A Laboratory Manual. Cold Spring Harbor Laboratory Press, Cold Spring Harbor, N.Y.). These include, but are not limited to, the hybridoma technique originally developed by Kohler and Milstein (Kohler and Milstein, Nature 256:495-497 (1975)), as well as the trioma technique, the human B-cell hybridoma technique (See e.g., Kozbor et al. Immunol. Today 4:72 (1983)), and the EBV-hybridoma technique to produce human monoclonal antibodies (Cole et al., in Monoclonal Antibodies and Cancer Therapy, Alan R. Liss, Inc., pp. 77-96 (1985)).

In an additional embodiment of the invention, monoclonal antibodies can be produced in germ-free animals utilizing recent technology (See e.g., PCT/US90/02545). According to the invention, human antibodies may be used and can be obtained by using human hybridomas (Cote et al., Proc. Natl. Acad. Sci. U.S.A. 80:2026-2030 (1983)) or by transforming human B cells with EBV virus in vitro (Cole et al., in Monoclonal Antibodies and Cancer Therapy, Alan R. Liss, pp. 77-96 (1985)).

According to the invention, techniques described for the production of single chain antibodies (see, e.g., U.S. Pat. No. 4,946,778) can be adapted to produce specific single chain antibodies. An additional embodiment of the invention utilizes the techniques described for the construction of Fab expression libraries (Huse et al., Science 246:1275-1281 (1989)) to allow rapid and easy identification of monoclonal Fab fragments with the desired specificity.

Antibody fragments that contain the idiotype (antigen binding region) of the antibody molecule can be generated by known techniques. For example, such fragments include but are not limited to: the F(ab′)2 fragment that can be produced by pepsin digestion of the antibody molecule; the Fab′ fragments that can be generated by reducing the disulfide bridges of the F(ab′)2 fragment, and the Fab fragments that can be generated by treating the antibody molecule with papain and a reducing agent.

In the production of antibodies, screening for the desired antibody can be accomplished by techniques known in the art (e.g., radioimmunoassay. ELISA (enzyme-linked immunosorbant assay), “sandwich” immunoassays, immunoradiometric assays, gel diffusion precipitin reactions, immunodiffusion assays, in situ immunoassays (using colloidal gold, enzyme or radioisotope labels, for example), Western Blots, precipitation reactions, agglutination assays (e.g., gel agglutination assays, hemagglutination assays, etc.), complement fixation assays, immunofluorescence assays, protein A assays, and immunoelectrophoresis assays, etc.).

The modular dendrimer nanoparticles having precise numbers of imaging agents are not limited to a particular manner of conjugation with an antibody. In some embodiments, the antibodies are configured to conjugate with a modular dendrimer nanoparticle having an antibody conjugation ligand. For example, in some embodiments, the antibody is configured to conjugate with a modular dendrimer nanoparticle via a linkage with the antibody conjugation ligand. The present invention is not limited to a particular configuration of the antibody which facilitates such a conjugation with modular dendrimer nanoparticle having an antibody conjugation ligand. In some embodiments, a modular dendrimer nanoparticle having precise numbers of imaging agents and an antibody conjugation ligand is introduced to one of the two carboxylic acid groups at the c-termini of the antibody Fc region. In some embodiments, the antibody Fc region is modified such that one or more of the c-termini have thereon a dendrimer conjugation ligand. In some embodiments, the antibody Fc region is modified such that both of the c-termini have thereon a dendrimer conjugation ligand. In some embodiments, the antibody Fc region is modified such one or more of the carboxylic groups at the c-termini are modified into dendrimer conjugation ligands. In some embodiments, the antibody Fc region is modified such that both of the carboxylic groups at the c-termini are modified into dendrimer conjugation ligands. The present invention is not limited to a particular type or kind of dendrimer conjugation ligand. In some embodiments, the dendrimer conjugation ligand is configured to facilitate conjugation with a modular dendrimer nanoparticle having precise numbers of imaging agents and an antibody conjugation ligand.

In some embodiments, the dendrimer conjugation ligand is configured to facilitate conjugation with a modular dendrimer nanoparticle having precise numbers of imaging agents and an antibody conjugation ligand through use of click chemistry (e.g., a 1,3-dipolar cycloaddition reaction). Click chemistry involves, for example, the coupling of two different moieties (e.g., a therapeutic agent and a functional group) (e.g., a first functional group and a second functional group) (e.g., a dendrimer conjugation ligand and an antibody conjugation ligand) via a 1,3-dipolar cycloaddition reaction between an alkyne moiety (or equivalent thereof) on the surface of the first moeity and an azide moiety (or equivalent thereof) (or any active end group such as, for example, a primary amine end group, a hydroxyl end group, a carboxylic acid end group, a thiol end group, etc.) on the second moiety. Click chemistry is an attractive coupling method because, for example, it can be performed with a wide variety of solvent conditions including aqueous environments. For example, the stable triazole ring that results from coupling the alkyne with the azide is frequently achieved at quantitative yields and is considered to be biologically inert (see, e.g., Rostovtsev, V. V.; et al., Angewandte Chemie-International Edition 2002, 41, (14), 2596; Wu, P.; et al., Angewandte Chemie-International Edition 2004, 43, (30), 3928-3932). As examples of antibody conjugation ligands include, but are not limited to, alkyne groups (e.g., cyclooctyne, fluorinated cyclooctyne, alkyne), in some embodiments, the dendrimer conjugation ligand is an azide group (e.g., for purposes of facilitating a 1,3-dipolar cycloaddition reaction between the dendrimer conjugation ligand and the antibody conjugation ligand). As such, in some embodiments, the antibody Fc region is modified such that both of the carboxylic groups at the c-termini are modified into azide groups.

The present invention is not limited to a having a particular number of modular dendrimer nanoparticles having precise numbers of imaging agents conjugated with an antibody. In some embodiments, one modular dendrimer nanoparticle having precise numbers of imaging agents is conjugated with an antibody. In some embodiments, two modular dendrimer nanoparticles having precise numbers of imaging agents are conjugated with an antibody. In some embodiments, one modular dendrimer nanoparticles having precise numbers of imaging agents is conjugated with an antibody at one antibody Fe region. In some embodiments, two modular dendrimer nanoparticles having precise numbers of imaging agents are conjugated with an antibody at each antibody Fc region. Indeed, embodiments wherein the modular dendrimer nanoparticles have between 1 and 8 imaging agent conjugation ligands ensures that antibodies conjugated with two of such modular dendrimer nanoparticles (having conjugated imaging agents) will have between 2 and 16 imaging agents (e.g., between 1 and 8 for each modular dendrimer nanoparticle conjugated to each antibody).

In certain embodiments, the present invention provides methods for imaging different antigens having varying abundance quantities in a manner wherein the detected imaging agent intensity is equated. For example, in some embodiments, different types of antigens have differing levels of in vivo or in vitro abundance. In such embodiments, antibodies directed to the higher abundance antigen are configured to be conjugated with modular dendrimer nanoparticles having fewer imaging agents (e.g., 2 imaging agents) than modular dendrimer nanoparticles conjugated with antibodies directed to the lower abundance antigen (e.g., 16 imaging agents). Such embodiments permit the equating of imaging agent intensity for antigens regardless of the abundance levels of such antigens.

Antibodies conjugated with modular dendrimer nanoparticles having precise numbers of imaging agents represent a significant improvement within imaging application. For example, by controlling both the number and position of imaging agents loaded to an antibody, antibodies conjugated with such modular dendrimer nanodevices achieve higher consistency and reliability than currently available reagents, and lead to more consistent and reliable results in biological experiments. Furthermore, because antibodies conjugated with such modular dendrimer nanodevices offer a range of a number of imaging agents per antibody (e.g., 2-16 imaging agents), researchers have the ability to balance the fluorescence levels of different targets in multi-dye experiments, even when very “dim” antibody targets such as CD19 or CD26L are involved. This superior loading range additionally improves sensitivity, a feature that is especially important for low abundance biomolecules. In addition, the quantitative labeling of antibody reagents permits subtle but reproducible differences in target quantities to be detected, for example, for morphogen gradients. In addition, the ease of use and reliability of the labeling process with modular dendrimer nanoparticles enables a significant number of researchers to consistently label primary antibodies with the dye and dye number of their choice, and to eliminate dependence on secondary antibodies.

For clinical applications the consistency and reliability of reagents is paramount, and antibodies conjugated with such modular dendrimer nanodevices having precise numbers of imaging agents greatly reduces the risk of incorrect diagnoses as the result of reagent variability. In addition, some clinical assays, such as those for AIDS, require multi-time point measurements and thus multiple lots of the antibody reagent; these inter-batch measurements are more reliable with antibodies conjugated with such modular dendrimer nanodevices having precise numbers of imaging agents, due to batch-to-batch consistency. Finally, because of the high dye loadings and increased sensitivity with antibodies conjugated with such modular dendrimer nanodevices having precise numbers of imaging agents, earlier detection of diseases and pre-disease states is facilitated, leading to improved treatment outcomes.

Antibodies conjugated with such modular dendrimer nanodevices having precise numbers of imaging agents provide additional benefits through increased efficiency in the manufacturing process, as every antibody can be labeled using the same method. For example, even if reagent manufacturers only used antibodies conjugated with such modular dendrimer nanodevices having precise numbers of imaging agents to replace current repertoire of labeled antibodies, antibodies conjugated with such modular dendrimer nanodevices having precise numbers of imaging agents permits the accomplishment more easily and with fewer resources. In addition, due to the modularity of the antibodies conjugated with such modular dendrimer nanodevices having precise numbers of imaging agents with respect to both imaging agents and number of imaging agents, manufacturers have the option to easily conjugate any of a wide range of dyes—in different defined quantities—using the same universal reaction scheme.

In some embodiments, the modular dendrimer nanoparticles comprise additional functional agents (e.g., targeting agents, therapeutic agents, trigger agents, and additional imaging agents). The present invention is not limited to particular method for conjugating modular dendrimer nanoparticles with additional functional agents (see, e.g., U.S. Pat. Nos. 6,471,968, 7,078,461; U.S. patent application Ser. Nos. 09/940,243, 10/431,682, 11,503,742, 11,661,465, 11/523,509, 12/403,179, 12/106,876, 11/827,637, 10/039,393, 10/254,126, 09/867,924, 12/570,977, and 12/645,081; U.S. Provisional Patent Application Ser. Nos. 61/256,699, 61/226,993, 61/140,480, 61/091,608, 61/097,780, 61/101,461, 61/251,244, 60/604,321, 60/690,652, 60/707,991, 60/208,728, 60/718,448, 61/035,949, 60/830,237, and 60/925,181; and International Patent Application Nos. PCT/US2010/051835, PCT/US2010/050893; PCT/US2010/042556, PCT/US2001/015204, PCT/US2005/030278, PCT/US2009/069257, PCT/US2009/036992, PCT/US2009/059071, PCT/US2007/015976, and PCT/US2008/061023).

In some embodiments, conjugation between a modular dendrimer nanoparticle (e.g., a terminal arm of a dendrimer) and an additional functional ligand is accomplished during a “one-pot” reaction. The term “one-pot synthesis reaction” or equivalents thereof, e.g., “1-pot”, “one pot”, etc., refers to a chemical synthesis method in which all reactants are present in a single vessel. Reactants may be added simultaneously or sequentially, with no limitation as to the duration of time elapsing between introduction of sequentially added reactants. In some embodiments, a one-pot reaction occurs wherein a hydroxyl-terminated dendrimer (e.g., HO-PAMAM dendrimer) is reacted with one or more functional ligands (e.g., a therapeutic agent, a pro-drug, a trigger agent, a targeting agent, an imaging agent) in one vessel, such conjugation being facilitated by ester coupling agents (e.g., 2-chloro-1-methylpyridinium iodide and 4-(dimethylamino)pyridine) (see, e.g., U.S. Provisional Patent App. No. 61/226,993).

In some embodiments, conjugation between a modular dendrimer nanoparticle (e.g., a terminal arm of a dendrimer) and an additional functional ligand is accomplished via a 1,3-dipolar cycloaddition reaction (“click chemistry”). Click chemistry involves, for example, the coupling of two different moieties (e.g., a therapeutic agent and a functional group) (e.g., a first functional group and a second functional group) via a 1,3-dipolar cycloaddition reaction between an alkyne moiety (or equivalent thereof) on the surface of the first moeity and an azide moiety (or equivalent thereof) (or any active end group such as, for example, a primary amine end group, a hydroxyl end group, a carboxylic acid end group, a thiol end group, etc.) on the second moiety. Click chemistry is an attractive coupling method because, for example, it can be performed with a wide variety of solvent conditions including aqueous environments. For example, the stable triazole ring that results from coupling the alkyne with the azide is frequently achieved at quantitative yields and is considered to be biologically inert (see. e.g., Rostovtsev, V. V.; et al., Angewandte Chemie-International Edition 2002, 41, (14), 2596; Wu. P.; et al., Angewandte Chemie-International Edition 2004, 43, (30), 3928-3932).

In some embodiments, the additional functional group(s) is attached with the modular dendrimer nanoparticle via a linker. The present invention is not limited to a particular type or kind of linker. In some embodiments, the linker comprises a spacer comprising between 1 and 8 straight or branched carbon chains. In some embodiments, the straight or branched carbon chains are unsubstituted. In some embodiments, the straight or branched carbon chains are substituted with alkyls.

In some embodiments, the additional functional agent is a therapeutic agent. A wide range of therapeutic agents find use with the present invention. In some embodiments, the therapeutic agents are effective in treating autoimmune disorders and/or inflammatory disorders (e.g., arthritis). Examples of such therapeutic agents include, but are not limited to, disease-modifying antirheumatic drugs (e.g., leflunomide, methotrexate, sulfasalazine, hydroxychloroquine), biologic agents (e.g., rituximab, infliximab, etanercept, adalimumab, golimumab), nonsteroidal anti-inflammatory drugs (e.g., ibuprofen, celecoxib, ketoprofen, naproxen, piroxicam, diclofenac), analgesics (e.g., acetaminophen, tramadol), immunomodulators (e.g., anakinra, abatacept), and glucocorticoids (e.g., prednisone, methylprednisone), TNF-α inhibitors (e.g., adalimumab, certolizumab pegol, etanercept, golimumab, infliximab). IL-1 inhibitors, and metalloprotease inhibitors. In some embodiments, the therapeutic agents include, but are not limited to, infliximab, adalimumab, etanercept, parenteral gold or oral gold.

In some embodiments, the therapeutic agent is an agent configured for treating rheumatoid arthritis. Examples of agents configured for treating rheumatoid arthritis include, but are not limited to, disease-modifying antirheumatic drugs (e.g., leflunomide, methotrexate, sulfasalazine, hydroxychloroquine), biologic agents (e.g., rituximab, infliximab, etanercept, adalimumab, golimumab), nonsteroidal anti-inflammatory drugs (e.g., ibuprofen, celecoxib, ketoprofen, naproxen, piroxicam, diclofenac), analgesics (e.g., acetaminophen, tramadol), immunomodulators (e.g., anakinra, abatacept), and glucocorticoids (e.g., prednisone, methylprednisone).

In some embodiments, the therapeutic agent is a pain relief agent. Examples of pain relief agents include, but are not limited to, analgesic drugs and respective antagonists. Examples of analgesic drugs include, but are not limited to, paracetamol and Non-steroidal anti-inflammatory drugs (NSAIDs), COX-2 inhibitors, opiates and morphonimimetics, and specific analgesic agents.

In some embodiments, the therapeutic agent includes, but is not limited to, a chemotherapeutic agent, an anti-oncogenic agent, an anti-angiogenic agent, a tumor suppressor agent, and/or an anti-microbial agent, although the present invention is not limited by the nature of the therapeutic agent.

In some embodiments, the chemotherapeutic agent is selected from a group consisting of, but not limited to, platinum complex, verapamil, podophylltoxin, carboplatin, procarbazine, mechloroethamine, cyclophosphamide, camptothecin, ifosfamide, melphalan, chlorambucil, bisulfan, nitrosurea, adriamycin, dactinomycin, daunorubicin, doxorubicin, bleomycin, plicomycin, mitomycin, bleomycin, etoposide, tamoxifen, paclitaxel, taxol, transplatinum, 5-fluorouracil, vincristin, vinblastin, bisphosphonate (e.g., CB3717), chemotherapeutic agents with high affinity for folic acid receptors, ALIMTA (Eli Lilly), and methotrexate.

Examples of anti-angiogenic agents include, but not limited to, Batimastat, Marimastat, AG3340, Neovastat, PEX, TIMP-1, -2, -3, -4, PAI-1, -2, uPA Ab, uPAR Ab, Amiloride, Minocycline, tetracyclines, steroids, cartilage-derived TIMP, αvβ3 Ab: LM609 and Vitaxin, RGD containing peptides, αvβ5 Ab, Endostatin, Angiostatin, aaAT, IFN-α, IFN-γ, IL-12, nitric oxide synthase inhibitors, TSP-1, TNP-470, Combretastatin A4, Thalidomide, Linomide, IFN-α, PF-4, prolactin fragment, Suramin and analogues, PPS, distamycin A analogues, FGF-2 Ab, antisense-FGF-2, Protamine, SU5416, soluble Flt-1, dominant-negative Flk-1, VEGF receptor ribosymes, VEGF Ab, Aspirin, NS-398, 6-AT, 6A5BU, 7-DX, Genistein, Lavendustin A, Ang-2, batimastat, marimastat, anti-αvβ3 monoclonal antibody (LM609) thrombospondin-1 (TSP-1) Angiostatin, endostatin, TNP-470, Combretastatin A-4, Anti-VEGF antibodies, soluble Flk-1, Fit-1 receptors, inhibitors of tyrosine kinase receptors, SU5416, heparin-binding growth factors, pentosan polysulfate, platelet-derived endothelial cell growth factor/Thymidine phosphorylase (PD-ECGF/TP), cox (e.g., cox-1 an cox-2) inhibitors (e.g., Celebrex and Vioxx), DT385, Tissue inhibitor of metalloprotease (TIMP-1, TIMP-2), Zinc, Plasminogen activator-inhibitor-1 (PAI-1), p53 Rb, Interleukin-10 Interleukin-12, Angiopoietin-2, Angiotensin, Angiotensin II (AT2 receptor), Caveolin-1, caveolin-2, Angiopoictin-2, Angiotensin, Angiotensin II (AT2 receptor), Caveolin-1, caveolin-2, Endostatin, Interferon-alpha, Isoflavones, Platelet factor-4, Prolactin (16 Kd fragment), Thrombospondin, Troponin-1, Bay 12-9566, AG3340, CGS 27023A, CGS 27023A, COL-3, (Neovastat), BMS-275291, Penicillamine, TNP-470 (fumagillin derivative), Squalamine, Combretastatin, Endostatin, Penicillamine, Farnesyl Transferase Inhibitor (FTI), -L-778,123, -SCH66336, -R115777, anti-VEGF antibody, Thalidomide, SU5416, Ribozyme, Angiozyme, SU6668, PTK787/ZK22584, Interferon-alpha, Interferon-alpha, Suramin, Vitaxin, EMD121974, Penicillamine, Tetrathiomolybdate, Captopril, serine protease inhibitors, CAI, ABT-627, CM101/ZDO101, Interleukin-12, IM862, PNU-145156E, those described in U.S. Patent App. No. 20050123605, and fragments or portions of the above that retain anti-angiogenic (e.g., angiostatic or inhibitory properties).

In some embodiments of the present invention, a dendrimer conjugate comprises one or more agents that directly cross-link nucleic acids (e.g., DNA) to facilitate DNA damage leading to, for example, synergistic, antineoplastic agents of the present invention. Agents such as cisplatin, and other DNA alkylating agents may be used. Cisplatin has been widely used to treat cancer, with efficacious doses used in clinical applications of 20 mg/M² for 5 days every three weeks for a total of three courses. The dendrimers may be delivered via any suitable method, including, but not limited to, injection intravenously, subcutaneously, intratumorally, intraperitoneally, or topically (e.g., to mucosal surfaces).

Agents that damage DNA also include compounds that interfere with DNA replication, mitosis and chromosomal segregation. Such chemotherapeutic compounds include adriamycin, also known as doxorubicin, etoposide, verapamil, podophyllotoxin, and the like. Widely used in a clinical setting for the treatment of neoplasms, these compounds are administered through bolus injections intravenously at doses ranging from 25-75 Mg/M² at 21 day intervals for adriamycin, to 35-50 Mg/M² for etoposide intravenously or double the intravenous dose orally.

Agents that disrupt the synthesis and fidelity of nucleic acid precursors and subunits also lead to DNA damage and find use as chemotherapeutic agents in the present invention. A number of nucleic acid precursors have been developed. Particularly useful are agents that have undergone extensive testing and are readily available. As such, agents such as 5-fluorouracil (5-FU) are preferentially used by neoplastic tissue, making this agent particularly useful for targeting to neoplastic cells. The doses delivered may range from 3 to 15 mg/kg/day, although other doses may vary considerably according to various factors including stage of disease, amenability of the cells to the therapy, amount of resistance to the agents and the like.

Photodynamic therapeutic agents may also be used as therapeutic agents in the present invention. In some embodiments, the dendrimer conjugates of the present invention containing photodynamic compounds are illuminated, resulting in the production of singlet oxygen and free radicals that diffuse out of the fiberless radiative effector to act on the biological target (e.g., tumor cells or bacterial cells).

Other photodynamic compounds useful in the present invention include those that cause cytotoxity by a different mechanism than singlet oxygen production (e.g., copper benzochlorin, Selman, et al., Photochem. Photobiol., 57:681-85 (1993). Examples of photodynamic compounds that find use in the present invention include, but are not limited to Photofrin 2, phtalocyanins (See e.g., Brasseur et al., Photochem. Photobiol., 47:705-11 (1988)), benzoporphyrin, tetrahydroxyphenylporphyrins, naphtalocyanines (See e.g., Firey and Rodgers, Photochem. Photobiol., 45:535-38 (1987)), sapphyrins (See. e.g., Sessler et al., Proc. SPIE, 1426:318-29 (1991)), porphinones (See, e.g., Chang et al., Proc. SPIE, 1203:281-86 (1990)), tin etiopurpurin, ether substituted porphyrins (See, e.g., Pandey et al., Photochem. Photobiol., 53:65-72 (1991)), and cationic dyes such as the phenoxazines (See e.g., Cincotta et al., SPIE Proc., 1203:202-10 (1990)).

In some embodiments, the therapeutic complexes of the present invention comprise a photodynamic compound and a targeting agent that is administered to a patient. In some embodiments, the targeting agent is then allowed a period of time to bind the “target” cell (e.g. about 1 minute to 24 hours) resulting in the formation of a target cell-target agent complex. In some embodiments, the therapeutic complexes comprising the targeting agent and photodynamic compound are then illuminated (e.g., with a red laser, incandescent lamp, X-rays, or filtered sunlight). In some embodiments, the light is aimed at the jugular vein or some other superficial blood or lymphatic vessel. In some embodiments, the singlet oxygen and free radicals diffuse from the photodynamic compound to the target cell (e.g. cancer cell or pathogen) causing its destruction.

In some embodiments, the therapeutic agent is conjugated to a trigger agent. The present invention is not limited to particular types or kinds of trigger agents.

In some embodiments, sustained release (e.g., slow release over a period of 24-48 hours) of the therapeutic agent is accomplished through conjugating the therapeutic agent (e.g., directly) (e.g., indirectly through one or more additional functional groups) to a trigger agent that slowly degrades in a biological system (e.g., amide linkage, ester linkage, ether linkage). In some embodiments, constitutively active release of the therapeutic agent is accomplished through conjugating the therapeutic agent to a trigger agent that renders the therapeutic agent constitutively active in a biological system (e.g., amide linkage, ether linkage).

In some embodiments, release of the therapeutic agent under specific conditions is accomplished through conjugating the therapeutic agent (e.g., directly) (e.g., indirectly through one or more additional functional groups) to a trigger agent that degrades under such specific conditions (e.g., through activation of a trigger molecule under specific conditions that leads to release of the therapeutic agent). For example, once a conjugate (e.g., a therapeutic agent conjugated with a trigger agent and a targeting agent) arrives at a target site in a subject (e.g., a tumor, or a site of inflammation), components in the target site (e.g., a tumor associated factor, or an inflammatory or pain associated factor) interact with the trigger agent thereby initiating cleavage of the therapeutic agent from the trigger agent. In some embodiments, the trigger agent is configured to degrade (e.g., release the therapeutic agent) upon exposure to a tumor-associated factor (e.g., hypoxia and pH, an enzyme (e.g., glucuronidase and/or plasmin), a cathepsin, a matrix metalloproteinase, a hormone receptor (e.g., integrin receptor, hyaluronic acid receptor, luteinizing hormone-releasing hormone receptor, etc.), cancer and/or tumor specific DNA sequence), an inflammatory associated factor (e.g., chemokine, cytokine, etc.) or other moiety.

In some embodiments, the present invention provides a therapeutic agent conjugated with a trigger agent that is sensitive to (e.g., is cleaved by) hypoxia (e.g., indolequinone). Hypoxia is a feature of several disease states, including cancer, inflammation and rheumatoid arthritis, as well as an indicator of respiratory depression (e.g., resulting from analgesic drugs).

Advances in the chemistry of bioreductive drug activation have led to the design of various hypoxia-selective drug delivery systems in which the pharmacophores of drugs are masked by reductively cleaved groups. In some embodiments, the trigger agent is utilizes a quinone, N-oxide and/or (hetero)aromatic nitro groups. For example, a quinone present in a conjugate is reduced to phenol under hypoxia conditions, with spontaneous formation of lactone that serves as a driving force for drug release. In some embodiments, a heteroaromatic nitro compound present in a conjugate (e.g., a therapeutic agent conjugated (e.g., directly or indirectly) with a trigger agent) is reduced to either an amine or a hydroxylamine, thereby triggering the spontaneous release of a therapeutic agent. In some embodiments, the trigger agent degrades upon detection of reduced pO₂ concentrations (e.g., through use of a redox linker).

The concept of pro-drug systems in which the pharmacophores of drugs are masked by reductively cleavable groups has been widely explored by many research groups and pharmaceutical companies (see, e.g., Beall, H. D., et al., Journal of Medicinal Chemistry, 1998. 41(24): p. 4755-4766; Ferrer. S., D. P. Naughton, and M. D. Threadgill, Tetrahedron, 2003. 59(19): p. 3445-3454; Naylor. M. A., et al., Journal of Medicinal Chemistry, 1997. 40(15): p. 2335-2346; Phillips, R. M., et al., Journal of Medicinal Chemistry, 1999. 42(20): p. 4071-4080; Zhang, Z., et al., Organic & Biomolecular Chemistry, 2005. 3(10): p. 1905-1910). Several such hypoxia activated pro-drugs have been advanced to clinical investigations, and work in relevant oxygen concentrations to prevent cerebral damage. The present invention is not limited to particular hypoxia-activated trigger agents. In some embodiments, the hypoxia-activated trigger agents include, but are not limited to, indolequinones, nitroimidazoles, and nitroheterocycles (see, e.g., Damen, E. W. P., et al., Bioorganic & Medicinal Chemistry, 2002. 10(1): p. 71-77; Hay, M. P., et al., Journal of Medicinal Chemistry, 2003. 46(25): p. 5533-5545; Hay, M. P., et al., Journal of the Chemical Society-Perkin Transactions 1, 1999(19): p. 2759-2770).

In some embodiments, the trigger agent is sensitive to (e.g., is cleaved by) and/or associates with a tumor-associated enzyme. For example, in some embodiments, the trigger agent that is sensitive to (e.g., is cleaved by) and/or associates with a glucuronidase. Glucuronic acid can be attached to several anticancer drugs via various linkers. These anticancer drugs include, but are not limited to, doxorubicin, paclitaxel, docetaxel, 5-fluorouracil, 9-aminocamtothecin, as well as other drugs under development. These pro-drugs are generally stable at physiological pH and are significantly less toxic than the parent drugs.

In some embodiments, the trigger agent is sensitive to (e.g., is cleaved by) and/or associates with brain enzymes. For example, trigger agents such as indolequinone are reduced by brain enzymes such as, for example, diaphorase (DT-diaphorase) (see. e.g., Damen, E. W. P., et al., Bioorganic & Medicinal Chemistry, 2002. 10(1): p. 71-77). For example, in such embodiments, the antagonist is only active when released during hypoxia to prevent respiratory failure.

In some embodiments, the trigger agent is sensitive to (e.g., is cleaved by) and/or associates with a protease. The present invention is not limited to any particular protease. In some embodiments, the protease is a cathepsin. In some embodiments, a trigger comprises a Lys-Phe-PABC moiety (e.g., that acts as a trigger). In some embodiments, a Lys-Phe-PABC moiety linked to doxorubicin, mitomycin C, and paclitaxel are utilized as a trigger-therapeutic conjugate in a conjugated dendrimer provided herein (e.g., that serve as substrates for lysosomal cathepsin B or other proteases expressed (e.g., overexpressed) in tumor cells). In some embodiments, utilization of a 1,6-elimination spacer/linker is utilized (e.g., to permit release of therapeutic drug post activation of trigger).

In some embodiments, the trigger agent is sensitive to (e.g., is cleaved by) and/or associates with plasmin. The serine protease plasmin is over expressed in many human tumor tissues. Tripeptide specifiers (e.g., including, but not limited to, Val-Leu-Lys) have been identified and linked to anticancer drugs through elimination or cyclization linkers.

In some embodiments, the trigger agent is sensitive to (e.g., is cleaved by) and/or associates with a matrix metalloprotease (MMP). In some embodiments, the trigger agent is sensitive to (e.g., is cleaved by) and/or that associates with β-Lactamase (e.g., a β-Lactamase activated cephalosporin-based pro-drug).

In some embodiments, the trigger agent is sensitive to (e.g., is cleaved by) and/or activated by a receptor (e.g., expressed on a target cell (e.g., a tumor cell)).

In some embodiments, the trigger agent that is sensitive to (e.g., is cleaved by) and/or activated by a nucleic acid. Nucleic acid triggered catalytic drug release can be utilized in the design of chemotherapeutic agents. Thus, in some embodiments, disease specific nucleic acid sequence is utilized as a drug releasing enzyme-like catalyst (e.g., via complex formation with a complimentary catalyst-bearing nucleic acid and/or analog). In some embodiments, the release of a therapeutic agent is facilitated by the therapeutic component being attached to a labile protecting group, such as, for example, cisplatin or methotrexate being attached to a photolabile protecting group that becomes released by laser light directed at cells emitting a color of fluorescence (e.g., in addition to and/or in place of target activated activation of a trigger component of a conjugated dendrimer of the present invention. In some embodiments, the therapeutic device also may have a component to monitor the response of the tumor to therapy. For example, where a therapeutic agent of the dendrimer induces apoptosis of a target cell (e.g., a cancer cell (e.g., a prostate cancer cell)), the caspase activity of the cells may be used to activate a green fluorescence. This allows apoptotic cells to turn orange, (combination of red and green) while residual cells remain red. Any normal cells that are induced to undergo apoptosis in collateral damage fluoresce green.

In some embodiments, in addition to antibodies, the modular dendrimer nanoparticles further comprise a targeting agent. For example, in some embodiments, a number of different expressed cell surface receptors find use as targets for the binding and uptake of a dendrimer conjugate. Such receptors include, but are not limited to, EGF receptor, folate receptor, FGR receptor 2, and the like.

FA has a high affinity for the folate receptor which is overexpressed in many epithelial cancer cells, including breast, ovary, endometrium, kidney, lung, head and neck, brain, and myeloid cancers (Weitman et al. (1992) Cancer Res. 52:6708-6711; Campbell et al. (1991) Cancer Res. 51:5329-5338; Weitman et al. (1992) Cancer Res. 73:2432-2443; Ross et al. (1994) Cancer 73:2432-2443), and is internalized into cells after ligand binding (Antony et al. (1985) J. Biol. Chem. 260:4911-4917). Tumor-selective targeting has been achieved by FA-conjugated liposomes encapsulting an antineoplastic drug (Lee et al. (1995) Bioochem. Biophys. Acta-Biomembranes 1233:134-144) or an antisense olignucleotides (Wang et al. (1995) PNAS 92:3318-3322), FA-conjugated protein toxin (Leamon et al. (1994) J. Drug Targeting 2:101-112), and FA-derivatized antibodies or their Fab/scFv fragments binding to the T-cell receptor (Rund et al. (1999) Intl. J. Cancer 83:141-149). In vivo studies have shown that the administration of multivalent, folate-targeted dendrimer-methotrexate conjugates resulted in significantly lower toxicity and a ten-fold enhancement in efficacy compared to free methotrexate at an equal cumulative dose (see, e.g., Kukowska-Latallo et al. (2005) Cancer Res. 65:5317-5324; Hong et al. (2007) Chem. & Biol. 14:107-115).

In some embodiments of the present invention, changes in gene expression associated with chromosomal abborations are the signature component. For example, Burkitt lymphoma results from chromosome translocations that involve the Myc gene. A chromosome translocation means that a chromosome is broken, which allows it to associate with parts of other chromosomes. The classic chromosome translocation in Burkitt lymophoma involves chromosome 8, the site of the Myc gene. This changes the pattern of Myc expression, thereby disrupting its usual function in controlling cell growth and proliferation.

In other embodiments, gene expression associated with colon cancer are identified as the signature component. Two key genes are known to be involved in colon cancer: MSH2 on chromosome 2 and MLH1 on chromosome 3. Normally, the protein products of these genes help to repair mistakes made in DNA replication. If the MSH2 and MLH1 proteins are mutated, the mistakes in replication remain unrepaired, leading to damaged DNA and colon cancer. MEN1 gene, involved in multiple endocrine neoplasia, has been known for several years to be found on chromosome 11, was more finely mapped in 1997, and serves as a signature for such cancers. In preferred embodiments of the present invention, an antibody specific for the altered protein or for the expressed gene to be detected is complexed with nanodevices of the present invention.

In yet another embodiment, adenocarcinoma of the colon has defined expression of CEA and mutated p53, both well-documented tumor signatures. The mutations of p53 in some of these cell lines are similar to that observed in some of the breast cancer cells and allows for the sharing of a p53 sensing component between the two nanodevices for each of these cancers (i.e., in assembling the nanodevice, dendrimers comprising the same signature identifying agent may be used for each cancer type). Both colon and breast cancer cells may be reliably studied using cell lines to produce tumors in nude mice, allowing for optimization and characterization in animals.

From the discussion above it is clear that there are many different tumor signatures that find use with the present invention, some of which are specific to a particular type of cancer and others which are promiscuous in their origin. The present invention is not limited to any particular tumor signature or any other disease-specific signature. For example, tumor suppressors that find use as signatures in the present invention include, but are not limited to, p53, Mucl, CEA, p16, p21, p27, CCAM, RB, APC, DCC, NF-1, NF-2, WT-1, MEN-1, MEN-II, p73, VHL, FCC and MCC.

In some embodiments, targeting agents are conjugated to the therapeutic agents for delivery of the dendrimer to desired body regions (e.g., to the central nervous system (CNS); to a tissue region associated with an inflammatory disorder and/or an autoimmune disorder (e.g., arthritis)). The targeting agents are not limited to targeting specific body regions.

In some embodiments, the targeting agent is a moiety that has affinity for a tumor associated factor. For example, a number of targeting agents are contemplated to be useful in the present invention including, but not limited to, RGD sequences, low-density lipoprotein sequences, a NAALADase inhibitor, epidermal growth factor, and other agents that bind with specificity to a target cell (e.g., a cancer cell)).

The present invention is not limited to cancer and/or tumor targeting agents. Indeed, conjugated dendrimers of the present invention can be targeted (e.g., via a linker conjugated to the dendrimer wherein the linker comprises a targeting agent) to a variety of target cells or tissues (e.g., to a biologically relevant environment) via conjugation to an appropriate targeting agent. For example, in some embodiments, the targeting agent is a moiety that has affinity for an inflammatory factor (e.g., a cytokine or a cytokine receptor moiety (e.g., TNF-α receptor)). In some embodiments, the targeting agent is a sugar, peptide, antibody or antibody fragment, hormone, hormone receptor, or the like.

In some embodiments of the present invention, the targeting agent includes but is not limited to an antibody, receptor ligand, hormone, vitamin, and antigen; however, the present invention is not limited by the nature of the targeting agent. In some embodiments, the antibody is specific for a disease-specific antigen. In some embodiments, the disease-specific antigen comprises a tumor-specific antigen. In some embodiments, the receptor ligand includes, but is not limited to, a ligand for CFTR, EGFR, estrogen receptor. FGR2, folate receptor, IL-2 receptor, glycoprotein, and VEGFR. In some embodiments, the receptor ligand is folic acid.

In some embodiments of the present invention, targeting groups are conjugated to dendrimers and/or linkers conjugated to the dendrimers with either short (e.g., direct coupling), medium (e.g. using small-molecule bifunctional linkers such as SPDP, sold by PIERCE CHEMICAL Company), or long (e.g., PEG bifunctional linkers, sold by NEKTAR, Inc.) linkages. Since dendrimers have surfaces with a large number of functional groups, more than one targeting group and/or linker may be attached to each dendrimer. As a result, multiple binding events may occur between the dendrimer conjugate and the target cell. In these embodiments, the dendrimer conjugates have a very high affinity for their target cells via this “cooperative binding” or polyvalent interaction effect. In preferred embodiments, at least two different ligand types are attached to the dendrimer, with or without linkers. In particularly preferred embodiments, the two different ligands are attached to the dendrimer through ester bonds.

For steric reasons, in some embodiments, the smaller the ligands, the more can be attached to the surface of a dendrimer and/or linkers attached thereto. Recently, Wiener reported that dendrimers with attached folic acid would specifically accumulate on the surface and within tumor cells expressing the high-affinity folate receptor (hFR) (See. e.g., Wiener et al., Invest. Radiol., 32:748 (1997)). The hFR receptor is expressed or upregulated on epithelial tumors, including breast cancers. Control cells lacking hFR showed no significant accumulation of folate-derivatized dendrimers. Folic acid can be attached to full generation PAMAM dendrimers via a carbodiimide coupling reaction. Folic acid is a good targeting candidate for the dendrimers, with its small size and a simple conjugation procedure.

In some embodiments, the targeting agents target the central nervous system (CNS). In some embodiments, where the targeting agent is specific for the CNS, the targeting agent is transferrin (see, e.g., Daniels, T. R., et al., Clinical Immunology, 2006. 121(2): p. 159-176; Daniels, T. R., et al., Clinical Immunology, 2006. 121(2): p. 144-158). Transferrin has been utilized as a targeting vector to transport, for example, drugs, liposomes and proteins across the blood-brain barrier (BBB) by receptor mediated transcytosis (see, e.g., Smith, M. W. and M. Gumbleton, Journal of Drug Targeting, 2006. 14(4): p. 191-214). In some embodiments, the targeting agents target neurons within the central nervous system (CNS). In some embodiments, where the targeting agent is specific for neurons within the CNS, the targeting agent is a synthetic tetanus toxin fragment (e.g., a 12 amino acid peptide (Tet 1) (HLNILSTLWKYR)) (SEQ ID NO: 2) (see, e.g., Liu, J. K., et al., Neurobiology of Disease, 2005. 19(3): p. 407-418).

In some embodiments of the present invention, additional imaging is based on the passive or active observation of local differences in density of selected physical properties of the investigated complex matter. These differences may be due to a different shape (e.g., mass density detected by atomic force microscopy), altered composition (e.g. radiopaques detected by X-ray), distinct light emission (e.g., fluorochromes detected by spectrophotometry), different diffraction (e.g., electron-beam detected by TEM), contrasted absorption (e.g., light detected by optical methods), or special radiation emission (e.g., isotope methods), etc. Thus, quality and sensitivity of imaging depend on the property observed and on the technique used. The imaging techniques for cancerous cells have to provide sufficient levels of sensitivity to allow observation of small, local concentrations of selected cells. The earliest identification of cancer signatures requires high selectivity (i.e., highly specific recognition provided by appropriate targeting) and the highest possible sensitivity.

Dendrimers have already been employed as biomedical imaging agents, perhaps most notably for magnetic resonance imaging (MRI) contrast enhancement agents (See e.g., Wiener et al., Mag. Reson. Med. 31:1 (1994); an example using PAMAM dendrimers). These agents are typically constructed by conjugating chelated paramagnetic ions, such as Gd(III)-diethylenetriaminepentaacetic acid (Gd(III)-DTPA), to water-soluble dendrimers. Other paramagnetic ions that may be useful in this context include, but are not limited to, gadolinium, manganese, copper, chromium, iron, cobalt, erbium, nickel, europium, technetium, indium, samarium, dysprosium, ruthenium, ytterbium, yttrium, and holmium ions and combinations thereof. In some embodiments of the present invention, a dendrimer conjugate is also conjugated to a targeting group, such as epidermal growth factor (EGF), to make the conjugate specifically bind to the desired cell type (e.g., in the case of EGF, EGFR-expressing tumor cells). In a preferred embodiment of the present invention, DTPA is attached to dendrimers via the isothiocyanate of DTPA as described by Wiener (Wiener et al., Mag. Reson. Med. 31:1 (1994)).

Dendrimeric MRI agents are particularly effective due to the polyvalency, size and architecture of dendrimers, which results in molecules with large proton relaxation enhancements, high molecular relativity, and a high effective concentration of paramagnetic ions at the target site. Dendrimeric gadolinium contrast agents have even been used to differentiate between benign and malignant breast tumors using dynamic MRI, based on how the vasculature for the latter type of tumor images more densely (Adam et al., Ivest. Rad. 31:26 (1996)). Thus, MRI provides a particularly useful imaging system of the present invention.

Some modular dendrimer nanoparticles of the present invention allow functional microscopic imaging of tumors and provide improved methods for imaging. The methods find use in vivo, in vitro, and ex vivo. For example, in one embodiment of the present invention, modular dendrimer nanoparticles of the present invention are designed to emit light or other detectable signals upon exposure to light. Although the labeled modular dendrimer nanoparticles may be physically smaller than the optical resolution limit of the microscopy technique, they become self-luminous objects when excited and are readily observable and measurable using optical techniques. In some embodiments of the present invention, sensing fluorescent biosensors in a microscope involves the use of tunable excitation and emission filters and multiwavelength sources (See, e.g., Farkas et al., SPEI 2678:200 (1997)). In embodiments where the imaging agents are present in deeper tissue, longer wavelengths in the Near-infrared (NMR) are used (See e.g., Lester et al., Cell Mol. Biol. 44:29 (1998)). Dendrimeric biosensing in the Near-IR has been demonstrated with dendrimeric biosensing antenna-like architectures (See, e.g., Shortreed et al., J. Phys. Chem., 101:6318 (1997)). Biosensors that find use with the present invention include, but are not limited to, fluorescent dyes and molecular beacons.

In some embodiments of the present invention, in vivo imaging is accomplished using functional imaging techniques. Functional imaging is a complementary and potentially more powerful technique as compared to static structural imaging. Functional imaging is best known for its application at the macroscopic scale, with examples including functional Magnetic Resonance Imaging (fMRI) and Positron Emission Tomography (PET). However, functional microscopic imaging may also be conducted and find use in in vivo and ex vivo analysis of living tissue. Functional microscopic imaging is an efficient combination of 3-D imaging, 3-D spatial multispectral volumetric assignment, and temporal sampling: in short a type of 3-D spectral microscopic movie loop. Interestingly, cells and tissues autofluoresce. When excited by several wavelengths, providing much of the basic 3-D structure needed to characterize several cellular components (e.g., the nucleus) without specific labeling. Oblique light illumination is also useful to collect structural information and is used routinely. As opposed to structural spectral microimaging, functional spectral microimaging may be used with biosensors, which act to localize physiologic signals within the cell or tissue. For example, in some embodiments of the present invention, biosensor-comprising dendrimers of the present invention are used to image upregulated receptor families such as the folate or EGF classes. In such embodiments, functional biosensing therefore involves the detection of physiological abnormalities relevant to carcinogenesis or malignancy, even at early stages. A number of physiological conditions may be imaged using the compositions and methods of the present invention including, but not limited to, detection of nanoscopic dendrimeric biosensors for pH, oxygen concentration, Ca²⁺ concentration, and other physiologically relevant analytes.

In some embodiments, the present invention provides modular dendrimer nanoparticles having a biological monitoring component. The biological monitoring or sensing component of a dendrimer is one that can monitor the particular response in a target cell (e.g., tumor cell) induced by an agent (e.g., a therapeutic agent provided by a conjugated dendrimer). While the present invention is not limited to any particular monitoring system, the invention is illustrated by methods and compositions for monitoring cancer treatments. In preferred embodiments of the present invention, the agent induces apoptosis in cells and monitoring involves the detection of apoptosis. In some embodiments, the monitoring component is an agent that fluoresces at a particular wavelength when apoptosis occurs. For example, in a preferred embodiment, caspase activity activates green fluorescence in the monitoring component. Apoptotic cancer cells, which have turned red as a result of being targeted by a particular signature with a red label, turn orange while residual cancer cells remain red. Normal cells induced to undergo apoptosis (e.g., through collateral damage), if present, will fluoresce green.

In these embodiments, fluorescent groups such as fluorescein are employed in the imaging agent. Fluorescein is easily attached to the dendrimer surface via the isothiocyanate derivatives, available from MOLECULAR PROBES, Inc. This allows the modular dendrimer nanoparticle to be imaged with the cells via confocal microscopy. Sensing of the effectiveness of modular dendrimer nanoparticle or components thereof is preferably achieved by using fluorogenic peptide enzyme substrates. For example, apoptosis caused by the therapeutic agent results in the production of the peptidase caspase-1 (ICE). CALBIOCHEM sells a number of peptide substrates for this enzyme that release a fluorescent moiety. A particularly useful peptide for use in the present invention is: MCA-Tyr-Glu-Val-Asp-Gly-Trp-Lys-(DNP)-NH₂ (SEQ ID NO: 1) where MCA is the (7-methoxycoumarin-4-yl)acetyl and DNP is the 2,4-dinitrophenyl group (See, e.g., Talanian et al., J. Biol. Chem., 272: 9677 (1997)). In this peptide, the MCA group has greatly attenuated fluorescence, due to fluorogenic resonance energy transfer (FRET) to the DNP group. When the enzyme cleaves the peptide between the aspartic acid and glycine residues, the MCA and DNP are separated, and the MCA group strongly fluoresces green (excitation maximum at 325 nm and emission maximum at 392 nm). In some embodiments, the lysine end of the peptide is linked to pro-drug complex, so that the MCA group is released into the cytosol when it is cleaved. The lysine end of the peptide is a useful synthetic handle for conjugation because, for example, it can react with the activated ester group of a bifunctional linker such as Mal-PEG-OSu. Thus the appearance of green fluorescence in the target cells produced using these methods provides a clear indication that apoptosis has begun (if the cell already has a red color from the presence of aggregated quantum dots, the cell turns orange from the combined colors).

Additional fluorescent dyes that find use with the present invention include, but are not limited to, acridine orange, reported as sensitive to DNA changes in apoptotic cells (see, e.g., Abrams et al., Development 117:29 (1993)) and cis-parinaric acid, sensitive to the lipid peroxidation that accompanies apoptosis (see, e.g., Hockenbery et al., Cell 75:241 (1993)). It should be noted that the peptide and the fluorescent dyes are merely exemplary. It is contemplated that any peptide that effectively acts as a substrate for a caspase produced as a result of apoptosis finds use with the present invention.

In some embodiments of the present invention, the lysine end of the peptide is linked to the modular dendrimer nanoparticle, so that the MCA group is released into the cytosol when it is cleaved. The lysine end of the peptide is a useful synthetic handle for conjugation because, for example, it can react with the activated ester group of a bifunctional linker such as Mal-PEG-OSu. Thus the appearance of green fluorescence in the target cells produced using these methods provides a clear indication that apoptosis has begun (if the cell already has a red color from the presence of aggregated quantum dots, the cell turns orange from the combined colors).

Additional fluorescent dyes that find use with the present invention include, but are not limited to, acridine orange, reported as sensitive to DNA changes in apoptotic cells (Abrams et al., Development 117:29 (1993)) and cis-parinaric acid, sensitive to the lipid peroxidation that accompanies apoptosis (Hockenbery et al., Cell 75:241 (1993)). It should be noted that the peptide and the fluorescent dyes are merely exemplary. It is contemplated that any peptide that effectively acts as a substrate for a caspase produced as a result of apoptosis finds use with the present invention.

As described above, another component of the present invention is that the dendrimer conjugate compositions are able to specifically target a particular cell type (e.g., tumor cell). In some embodiments, the dendrimer conjugate targets neoplastic cells through a cell surface moiety and is taken into the cell through receptor mediated endocytosis.

Where clinical applications are contemplated, in some embodiments of the present invention, the antibody//modular dendrimer nanoparticles are prepared as part of a pharmaceutical composition in a form appropriate for the intended application. Generally, this entails preparing compositions that are essentially free of pyrogens, as well as other impurities that could be harmful to humans or animals. However, in some embodiments of the present invention, a straight antibody//modular dendrimer nanoparticles formulation may be administered using one or more of the routes described herein.

In preferred embodiments, the antibody//modular dendrimer nanoparticles are used in conjunction with appropriate salts and buffers to render delivery of the compositions in a stable manner to allow for uptake by target cells. Buffers also are employed when the dendrimer conjugates are introduced into a patient. Aqueous compositions comprise an effective amount of the dendrimer conjugates to cells dispersed in a pharmaceutically acceptable carrier or aqueous medium. Such compositions also are referred to as inocula. The phrase “pharmaceutically or pharmacologically acceptable” refer to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. Except insofar as any conventional media or agent is incompatible with the vectors or cells of the present invention, its use in therapeutic compositions is contemplated. Supplementary active ingredients may also be incorporated into the compositions.

In some embodiments of the present invention, the active compositions include classic pharmaceutical preparations. Administration of these compositions according to the present invention is via any common route so long as the target tissue is available via that route. This includes oral, nasal, buccal, rectal, vaginal or topical. Alternatively, administration may be by orthotopic, intradermal, subcutaneous, intramuscular, intraperitoneal or intravenous injection.

The active antibody//modular dendrimer nanoparticles may also be administered parenterally or intraperitoneally or intratumorally. Solutions of the active compounds as free base or pharmacologically acceptable salts are prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

In some embodiments, a therapeutic agent is released from a antibody//modular dendrimer nanoparticle within a target cell (e.g., within an endosome). This type of intracellular release (e.g., endosomal disruption of a linker-therapeutic conjugate) is contemplated to provide additional specificity for the compositions and methods of the present invention. In some embodiments, the antibody//modular dendrimer nanoparticles of the present invention contain between 100-150 primary amines on the surface. Thus, the present invention provides dendrimers with multiple (e.g., 100-150) reactive sites for the conjugation of linkers and/or functional groups comprising, but not limited to, therapeutic agents, targeting agents, imaging agents and biological monitoring agents.

The compositions and methods of the present invention are contemplated to be equally effective whether or not the dendrimer conjugates of the present invention comprise a fluorescein (e.g. FITC) imaging agent. Thus, each functional group present in a dendrimer composition is able to work independently of the other functional groups. Thus, the present invention provides dendrimer conjugates that can comprise multiple combinations of targeting, therapeutic, imaging, and biological monitoring functional groups.

The present invention also provides a very effective and specific method of delivering molecules (e.g., therapeutic and imaging functional groups) to the interior of target cells (e.g., cancer cells). Thus, in some embodiments, the present invention provides methods of therapy that comprise or require delivery of molecules into a cell in order to function (e.g., delivery of genetic material such as siRNAs).

The pharmaceutical forms suitable for injectable use include sterile aqueous solutions or dispersions and sterile powders for the extemporaneous preparation of sterile injectable solutions or dispersions. The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial an antifungal agents, for example, parabens, chlorobutanol, phenol, sorbic acid, thimerosal, and the like. In many cases, it may be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Upon formulation, antibody//modular dendrimer nanoparticles are administered in a manner compatible with the dosage formulation and in such amount as is therapeutically effective. The formulations are easily administered in a variety of dosage forms such as injectable solutions, drug release capsules and the like. For parenteral administration in an aqueous solution, for example, the solution is suitably buffered, if necessary, and the liquid diluent first rendered isotonic with sufficient saline or glucose. These particular aqueous solutions are especially suitable for intravenous, intramuscular, subcutaneous and intraperitoneal administration. For example, one dosage could be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). In some embodiments of the present invention, the active particles or agents are formulated within a therapeutic mixture to comprise about 0.0001 to 1.0 milligrams, or about 0.001 to 0.1 milligrams, or about 0.1 to 1.0 or even about 10 milligrams per dose or so. Multiple doses may be administered.

Additional formulations that are suitable for other modes of administration include vaginal suppositories and pessaries. A rectal pessary or suppository may also be used. Suppositories are solid dosage forms of various weights and shapes, usually medicated, for insertion into the rectum, vagina or the urethra. After insertion, suppositories soften, melt or dissolve in the cavity fluids. In general, for suppositories, traditional binders and carriers may include, for example, polyalkylene glycols or triglycerides; such suppositories may be formed from mixtures containing the active ingredient in the range of 0.5% to 10%, preferably 1%-2%. Vaginal suppositories or pessaries are usually globular or oviform and weighing about 5 g each. Vaginal medications are available in a variety of physical forms, e.g., creams, gels or liquids, which depart from the classical concept of suppositories. In addition, suppositories may be used in connection with colon cancer. The dendrimer conjugates also may be formulated as inhalants for the treatment of lung cancer and such like.

It is contemplated that components of antibody//modular dendrimer nanoparticles of the present invention provide therapeutic benefits to patients suffering from medical conditions and/or diseases (e.g., cancer, inflammatory disease, chronic pain, autoimmune disease, etc.).

Indeed, in some embodiments of the present invention, methods and compositions are provided for the treatment of inflammatory diseases (e.g., antibody//modular dendrimer nanoparticles conjugated with therapeutic agents configured for treating inflammatory diseases). Inflammatory diseases include but are not limited to arthritis, rheumatoid arthritis, psoriatic arthritis, osteoarthritis, degenerative arthritis, polymyalgia rheumatic, ankylosing spondylitis, reactive arthritis, gout, pseudogout, inflammatory joint disease, systemic lupus erythematosus, polymyositis, and fibromyalgia. Additional types of arthritis include achilles tendinitis, achondroplasia, acromegalic arthropathy, adhesive capsulitis, adult onset Still's disease, anserine bursitis, avascular necrosis, Behcet's syndrome, bicipital tendinitis, Blount's disease, brucellar spondylitis, bursitis, calcaneal bursitis, calcium pyrophosphate dihydrate deposition disease (CPPD), crystal deposition disease, Caplan's syndrome, carpal tunnel syndrome, chondrocalcinosis, chondromalacia patellae, chronic synovitis, chronic recurrent multifocal osteomyelitis, Churg-Strauss syndrome, Cogan's syndrome, corticosteroid-induced osteoporosis, costosternal syndrome, CREST syndrome, cryoglobulinemia, degenerative joint disease, dermatomyositis, diabetic finger sclerosis, diffuse idiopathic skeletal hyperostosis (DISH), discitis, discoid lupus erythematosus, drug-induced lupus, Duchenne's muscular dystrophy, Dupuytren's contracture, Ehlers-Danlos syndrome, enteropathic arthritis, epicondylitis, erosive inflammatory osteoarthritis, exercise-induced compartment syndrome. Fabry's disease, familial Mediterranean fever. Farber's lipogranulomatosis, Felty's syndrome, Fifth's disease, flat feet, foreign body synovitis, Freiberg's disease, fungal arthritis, Gaucher's disease, giant cell arteritis, gonococcal arthritis, Goodpasture's syndrome, granulomatous arteritis, hemarthrosis, hemochromatosis. Henoch-Schonlein purpura, Hepatitis B surface antigen disease, hip dysplasia, Hurler syndrome, hypermobility syndrome, hypersensitivity vasculitis, hypertrophic osteoarthropathy, immune complex disease, impingement syndrome, Jaccoud's arthropathy, juvenile ankylosing spondylitis, juvenile dermatomyositis, juvenile rheumatoid arthritis, Kawasaki disease, Kienbock's disease, Legg-Calve-Perthes disease, Lesch-Nyhan syndrome, linear scleroderma, lipoid dermatoarthritis, Lofgren's syndrome, Lyme disease, malignant synovioma, Marfan's syndrome, medial plica syndrome, metastatic carcinomatous arthritis, mixed connective tissue disease (MCTD), mixed cryoglobulinemia, mucopolysaccharidosis, multicentric reticulohistiocytosis, multiple epiphyseal dysplasia, mycoplasmal arthritis, myofascial pain syndrome, neonatal lupus, neuropathic arthropathy, nodular panniculitis, ochronosis, olecranon bursitis, Osgood-Schlatter's disease, osteoarthritis, osteochondromatosis, osteogenesis imperfecta, osteomalacia, osteomyelitis, osteonecrosis, osteoporosis, overlap syndrome, pachydermoperiostosis Paget's disease of bone, palindromic rheumatism, patellofemoral pain syndrome, Pellegrini-Stieda syndrome, pigmented villonodular synovitis, piriformis syndrome, plantar fasciitis, polyarteritis nodos, Polymyalgia rheumatic, polymyositis, popliteal cysts, posterior tibial tendinitis, Pott's disease, prepatellar bursitis, prosthetic joint infection, pseudoxanthoma elasticum, psoriatic arthritis, Raynaud's phenomenon, reactive arthritis/Reiter's syndrome, reflex sympathetic dystrophy syndrome, relapsing polychondritis, retrocalcaneal bursitis, rheumatic fever, rheumatoid vasculitis, rotator cuff tendinitis, sacroiliitis, salmonella osteomyelitis, sarcoidosis, saturnine gout, Scheuermann's osteochondritis, scleroderma, septic arthritis, seronegative arthritis, shigella arthritis, shoulder-hand syndrome, sickle cell arthropathy, Sjogren's syndrome, slipped capital femoral epiphysis, spinal stenosis, spondylolysis, staphylococcus arthritis, Stickler syndrome, subacute cutaneous lupus, Sweet's syndrome, Sydenham's chorea, syphilitic arthritis, systemic lupus erythematosus (SLE), Takayasu's arteritis, tarsal tunnel syndrome, tennis elbow. Tietse's syndrome, transient osteoporosis, traumatic arthritis, trochanteric bursitis, tuberculosis arthritis, arthritis of Ulcerative colitis, undifferentiated connective tissue syndrome (UCTS), urticarial vasculitis, viral arthritis, Wegener's granulomatosis, Whipple's disease, Wilson's disease, and yersinial arthritis.

In some embodiments, antibody//modular dendrimer nanoparticles of the present invention configured for treating autoimmune disorders and/or inflammatory disorders (e.g., rheumatoid arthritis) are co-administered to a subject (e.g., a human suffering from an autoimmune disorder and/or an inflammatory disorder) a therapeutic agent configured for treating autoimmune disorders and/or inflammatory disorders (e.g., rheumatoid arthritis). Examples of such agents include, but are not limited to, disease-modifying antirheumatic drugs (e.g., leflunomide, methotrexate, sulfasalazine, hydroxychloroquine), biologic agents (e.g., rituximab, infliximab, etanercept, adalimumab, golimumab), nonsteroidal anti-inflammatory drugs (e.g., ibuprofen, celecoxib, ketoprofen, naproxen, piroxicam, diclofenac), analgesics (e.g., acetaminophen, tramadol), immunomodulators (e.g., anakinra, abatacept), and glucocorticoids (e.g., prednisone, methylprednisone).

In some embodiments, the medical condition and/or disease is pain (e.g., chronic pain, mild pain, recurring pain, severe pain, etc.). In some embodiments, the conjugated dendrimers of the present invention are configured to deliver pain relief agents to a subject. In some embodiments, the dendrimer conjugates are configured to deliver pain relief agents and pain relief agent antagonists to counter the side effects of pain relief agents. The dendrimer conjugates are not limited to treating a particular type of pain and/or pain resulting from a disease. Examples include, but are not limited to, pain resulting from trauma (e.g., trauma experienced on a battlefield, trauma experienced in an accident (e.g., car accident)). In some embodiments, the dendrimer conjugates of the present invention are configured such that they are readily cleared from the subject (e.g., so that there is little to no detectable toxicity at efficacious doses).

In some embodiments, the disease is cancer. The present invention is not limited by the type of cancer treated using the compositions and methods of the present invention. Indeed, a variety of cancer can be treated including, but not limited to, prostate cancer, colon cancer, breast cancer, lung cancer and epithelial cancer. Similarly, the present invention is not limited by the type of inflammatory disease and/or chronic pain treated using the compositions of the present invention. Indeed, a variety of diseases can be treated including, but not limited to, arthritis (e.g., osteoarthritis, rheumatoid arthritis, etc.), inflammatory bowel disease (e.g., colitis, Crohn's disease, etc.), autoimmune disease (e.g., lupus erythematosus, multiple sclerosis, etc.), inflammatory pelvic disease, etc.

In some embodiments, the disease is a neoplastic disease, selected from, but not limited to, leukemia, acute leukemia, acute lymphocytic leukemia, acute myelocytic leukemia, myeloblastic, promyelocytic, myelomonocytic, monocytic, erythroleukemia, chronic leukemia, chronic myelocytic, (granulocytic) leukemia, chronic lymphocytic leukemia, Polycythemia vera, lymphoma, Hodgkin's disease, non-Hodgkin's disease, Multiple myeloma, Waldenstrom's macroglobulinemia, Heavy chain disease, solid tumors, sarcomas and carcinomas, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, uterine cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, and neuroblastomaretinoblastoma. In some embodiments, the disease is an inflammatory disease selected from the group consisting of, but not limited to, eczema, inflammatory bowel disease, rheumatoid arthritis, asthma, psoriasis, ischemia/reperfusion injury, ulcerative colitis and acute respiratory distress syndrome. In some embodiments, the disease is a viral disease selected from the group consisting of, but not limited to, viral disease caused by hepatitis B, hepatitis C, rotavirus, human immunodeficiency virus type I (HIV-I), human immunodeficiency virus type II (HIV-II), human T-cell lymphotropic virus type I (HTLV-I), human T-cell lymphotropic virus type II (HTLV-II), AIDS, DNA viruses such as hepatitis type B and hepatitis type C virus; parvoviruses, such as adeno-associated virus and cytomegalovirus; papovaviruses such as papilloma virus, polyoma viruses, and SV40; adenoviruses; herpes viruses such as herpes simplex type I (HSV-I), herpes simplex type II (HSV-II), and Epstein-Barr virus; poxviruses, such as variola (smallpox) and vaccinia virus; and RNA viruses, such as human immunodeficiency virus type I (HIV-I), human immunodeficiency virus type II (HIV-II), human T-cell lymphotropic virus type I (HTLV-I), human T-cell lymphotropic virus type II (HTLV-II), influenza virus, measles virus, rabies virus, Sendai virus, picornaviruses such as poliomyelitis virus, coxsackieviruses, rhinoviruses, reoviruses, togaviruses such as rubella virus (German measles) and Semliki forest virus, arboviruses, and hepatitis type A virus.

It is contemplated that the antibody//modular dendrimer nanoparticles of the present invention can be employed in the treatment of any pathogenic disease for which a specific signature has been identified or which can be targeted for a given pathogen. Examples of pathogens contemplated to be treatable with the methods of the present invention include, but are not limited to, Legionella peomophilia, Mycobacterium tuberculosis, Clostridium tetani, Hemophilus influenzae, Neisseria gonorrhoeae, Treponmema pallidum, Bacillus anthracis, Vibrio cholerae, Borrelia burgdorferi, Cornebacterium diphtheria, Staphylococcus aureus, human papilloma virus, human immunodeficiency virus, rubella virus, polio virus, and the like.

The present invention also includes methods involving co-administration of the antibody//modular dendrimer nanoparticles of the present invention with one or more additional active agents. Indeed, it is a further aspect of this invention to provide methods for enhancing prior art therapies and/or pharmaceutical compositions by co-administering conjugated dendrimers of this invention. In co-administration procedures, the agents may be administered concurrently or sequentially. In some embodiments, the conjugated dendrimers described herein are administered prior to the other active agent(s). The agent or agents to be co-administered depends on the type of condition being treated. For example, when the condition being treated is arthritis, the additional agent can be an agent effective in treating arthritis (e.g., TNF-α inhibitors such as anti-TNF α monoclonal antibodies (such as REMICADE®, CDP-870 and HUMIRA™ (adalimumab) and TNF receptor-immunoglobulin fusion molecules (such as ENBREL®)(entanercept), IL-1 inhibitors, receptor antagonists or soluble IL-1R a (e.g. KINERET™ or ICE inhibitors), nonsteroidal anti-inflammatory agents (NSAIDS), piroxicam, diclofenac, naproxen, flurbiprofen, fenoprofen, ketoprofen ibuprofen, fenamates, mefenamic acid, indomethacin, sulindac, apazone, pyrazolones, phenylbutazone, aspirin, COX-2 inhibitors (such as CELEBREX® (celecoxib), VIOXX® (rofecoxib), BEXTRA® (valdecoxib) and etoricoxib, (preferably MMP-13 selective inhibitors), NEUROTIN®, pregabalin, sulfasalazine, low dose methotrexate, leflunomide, hydroxychloroquine, d-penicillamine, auranofin or parenteral or oral gold). The additional agents to be co-administered can be any of the well-known agents in the art, including, but not limited to, those that are currently in clinical use. The determination of appropriate type and dosage of radiation treatment is also within the skill in the art or can be determined with relative ease.

In some embodiments, the composition is co-administered with an anti-cancer agent (e.g., Acivicin; Aclarubicin; Acodazole Hydrochloride; Acronine; Adozelesin; Adriamycin; Aldesleukin; Alitretinoin; Allopurinol Sodium; Altretamine; Ambomycin; Ametantrone Acetate; Aminoglutethimide; Amsacrine; Anastrozole; Annonaceous Acetogenins; Anthramycin; Asimicin; Asparaginase; Asperlin; Azacitidine; Azetepa; Azotomycin; Batimastat; Benzodepa; Bexarotene; Bicalutamide; Bisantrene Hydrochloride; Bisnafide Dimesylate; Bizelesin; Bleomycin Sulfate; Brequinar Sodium; Bropirimine; Bullatacin; Busulfan; Cabergoline; Cactinomycin; Calusterone; Caracemide; Carbetimer; Carboplatin; Carmustine; Carubicin Hydrochloride; Carzelesin; Cedefingol; Celecoxib; Chlorambucil; Cirolemycin; Cisplatin; Cladribine; Crisnatol Mesylate; Cyclophosphamide; Cytarabine; Dacarbazine; DACA (N-[2-(Dimethyl-amino)ethyl]acridine-4-carboxamide); Dactinomycin; Daunorubicin Hydrochloride; Daunomycin; Decitabine; Denileukin Diftitox; Dexormaplatin; Dezaguanine; Dezaguanine Mesylate; Diaziquone; Docetaxel; Doxorubicin; Doxorubicin Hydrochloride; Droloxifene; Droloxifene Citrate; Dromostanolone Propionate; Duazomycin; Edatrexate; Eflornithine Hydrochloride; Elsamitrucin; Enloplatin; Enpromate; Epipropidine; Epirubicin Hydrochloride; Erbulozole; Esorubicin Hydrochloride; Estramustine; Estramustine Phosphate Sodium; Etanidazole; Ethiodized Oil I 131; Etoposide; Etoposide Phosphate; Etoprine; Fadrozole Hydrochloride; Fazarabine; Fenretinide; Floxuridine; Fludarabine Phosphate; Fluorouracil; 5-FdUMP; Flurocitabine; Fosquidone; Fostriecin Sodium; FK-317; FK-973; FR-66979; FR-900482; Gemcitabine; Geimcitabine Hydrochloride; Gemtuzumab Ozogamicin; Gold Au 198; Goserelin Acetate; Guanacone; Hydroxyurea; Idarubicin Hydrochloride; Ifosfamide; Ilmofosine; Interferon Alfa-2a; Interferon Alfa-2b; Interferon Alfa-n1; Interferon Alfa-n3; Interferon Beta-la; Interferon Gamma-1b; Iproplatin; Irinotecan Hydrochloride; Lanreotide Acetate; Letrozole; Leuprolide Acetate; Liarozole Hydrochloride; Lometrexol Sodium; Lomustine; Losoxantrone Hydrochloride; Masoprocol; Maytansine; Mechlorethamine Hydrochloride; Megestrol Acetate; Melengestrol Acetate; Melphalan; Menogaril; Mercaptopurine; Methotrexate; Methotrexate Sodium; Methoxsalen; Metoprine; Meturedepa; Mitindomide; Mitocarcin; Mitocromin; Mitogillin; Mitomalcin; Mitomycin; Mytomycin C; Mitosper; Mitotane; Mitoxantrone Hydrochloride; Mycophenolic Acid; Nocodazole; Nogalamycin; Oprelvekin; Ormaplatin; Oxisuran; Paclitaxel; Pamidronate Disodium; Pegaspargase; Peliomycin; Pentamustine; Peplomycin Sulfate; Perfosfamide; Pipobroman; Piposulfan; Piroxantrone Hydrochloride; Plicamycin; Plomestane; Porfimer Sodium; Porfiromycin; Prednimustine; Procarbazine Hydrochloride; Puromycin; Puromycin Hydrochloride; Pyrazofurin; Riboprine; Rituximab; Rogletimide; Rolliniastatin; Safingol; Safingol Hydrochloride; Samarium/Lexidronam; Semustine; Simtrazene; Sparfosate Sodium; Sparsomycin; Spirogermanium Hydrochloride; Spiromustine; Spiroplatin; Squamocin; Squamotacin; Streptonigrin; Streptozocin; Strontium Chloride Sr 89; Sulofenur; Talisomycin; Taxane; Taxoid; Tecogalan Sodium; Tegafur; Teloxantrone Hydrochloride; Temoporfin; Teniposide; Teroxirone; Testolactone; Thiamiprine; Thioguanine; Thiotepa; Thymitaq; Tiazofurin; Tirapazamine; Tomudex; TOP-53; Topotecan Hydrochloride; Toremifene Citrate; Trastuzumab; Trestolone Acetate; Triciribine Phosphate; Trimetrexate; Trimetrexate Glucuronate; Triptorelin; Tubulozole Hydrochloride; Uracil Mustard; Uredepa; Valrubicin; Vapreotide; Verteporfin; Vinblastine; Vinblastine Sulfate; Vincristine; Vincristine Sulfate; Vindesine; Vindesine Sulfate; Vinepidine Sulfate; Vinglycinate Sulfate; Vinleurosine Sulfate; Vinorelbine Tartrate; Vinrosidine Sulfate; Vinzolidine Sulfate; Vorozole; Zeniplatin; Zinostatin; Zorubicin Hydrochloride; 2-Chlorodeoxyadenosine; 2′-Deoxyformycin; 9-aminocamptothecin; raltitrexed; N-propargyl-5,8-dideazafolic acid; 2-chloro-2′-arabino-fluoro-2′-deoxyadenosine; 2-chloro-2′-deoxyadenosine; anisomycin; trichostatin A; hPRL-G129R; CEP-751; linomide; sulfur mustard; nitrogen mustard (mechlorethamine); cyclophosphamide; melphalan; chlorambucil; ifosfamide; busulfan; N-methyl-N-nitrosourea (MNU); N,N′-Bis(2-chloroethyl)-N-nitrosourea (BCNU); N-(2-chloroethyl)-N′-cyclohex-yl-N-nitrosourea (CCNU); N-(2-chloroethyl)-N′-(trans-4-methylcyclohexyl-N-nitrosourea (MeCCNU); N-(2-chloroethyl)-N′-(diethyl)ethylphosphonate-N-nit-rosourea (fotemustine); streptozotocin; diacarbazine (DTIC); mitozolomide; temozolomide; thiotepa; mitomycin C; AZQ; adozelesin; Cisplatin; Carboplatin; Ormaplatin; Oxaliplatin; C1-973; DWA 2114R; JM216; JM335; Bis (platinum); tomudex; azacitidine; cytarabine; gemcitabine; 6-Mercaptopurine; 6-Thioguanine; Hypoxanthine; teniposide; 9-amino camptothecin; Topotecan; CPT-11; Doxorubicin; Daunomycin; Epirubicin; darubicin; mitoxantrone; losoxantrone; Dactinomycin (Actinomycin D); amsacrine; pyrazoloacridine; all-trans retinol; 14-hydroxy-retro-retinol; all-trans retinoic acid; N-(4-Hydroxyphenyl) retinamide; 13-cis retinoic acid; 3-Methyl TTNEB; 9-cis retinoic acid; fludarabine (2-F-ara-AMP); and 2-chlorodeoxyadenosine (2-Cda). Other anti-cancer agents include, but are not limited to, Antiproliferative agents (e.g., Piritrexim Isothionate), Antiprostatic hypertrophy agent (e.g., Sitogluside), Benign prostatic hyperplasia therapy agents (e.g., Tamsulosin Hydrochloride), Prostate growth inhibitor agents (e.g., Pentomone), and Radioactive agents; Fibrinogen I 125; Fludeoxyglucose F 18; Fluorodopa F 18; Insulin I 125; Insulin I 131; Iobenguane I 123; Iodipamide Sodium I 131; Iodoantipyrine I 131; Iodocholesterol I 131; Iodohippurate Sodium I 123; Iodohippurate Sodium I 125; Iodohippurate Sodium I 131; Iodopyracet I 125; Iodopyracet I 131; Iofetamine Hydrochloride I 123; Iomethin I 125; Iomethin I 131; lothalamate Sodium I 125; lothalamate Sodium I 131; Iotyrosine I 131; Liothyronine I 125; Liothyronine I 131; Merisoprol Acetate Hg 197; Merisoprol Acetate Hg 203; Merisoprol Hg 197; Selenomethionine Se 75; Technetium Tc 99m Antimony Trisulfide Colloid; Technetium Tc 99m Bicisate; Technetium Tc 99m Disofenin; Technetium Tc 99m Etidronate; Technetium Tc 99m Exametazime; Technetium Tc 99m Furifosmin; Technetium Tc 99m Gluceptate; Technetium Tc 99m Lidofenin; Technetium Tc 99m Mebrofenin; Technetium Tc 99m Medronate; Technetium Tc 99m Medronate Disodium; Technetium Tc 99m Mertiatide; Technetium Tc 99m Oxidronate; Technetium Tc 99m Pentetate; Technetium Tc 99m Pentetate Calcium Trisodium; Technetium Tc 99m Sestamibi; Technetium Tc 99m Siboroxime; Technetium Tc 99m Succimer; Technetium Tc 99m sulfur Colloid; Technetium Tc 99m Teboroxime; Technetium Tc 99m Tetrofosmin; Technetium Tc 99m Tiatide; Thyroxine I 125; Thyroxine I 131; Tolpovidone I 131; Triolein I 125; and Triolein I 131).

Additional anti-cancer agents include, but are not limited to anti-cancer Supplementary Potentiating Agents; Tricyclic anti-depressant drugs (e.g., imipramine, desipramine, amitryptyline, clomipramine, trimipramine, doxepin, nortriptyline, protriptyline, amoxapine and maprotiline); non-tricyclic anti-depressant drugs (e.g., sertraline, trazodone and citalopram); Ca⁺⁺ antagonists (e.g., verapamil, nifedipine, nitrendipine and caroverine); Calmodulin inhibitors (e.g., prenylamine, trifluoroperazine and clomipramine); Amphotericin B; Triparanol analogues (e.g., tamoxifen); antiarrhythmic drugs (e.g., quinidine); antihypertensive drugs (e.g., reserpine); Thiol depleters (e.g., buthionine and sulfoximine) and Multiple Drug Resistance reducing agents such as Cremaphor EL. Still other anticancer agents include, but are not limited to, annonaceous acetogenins; asimicin; rolliniastatin; guanacone, squamocin, bullatacin; squamotacin; taxanes; paclitaxel; gemcitabine; methotrexate FR-900482; FK-973; FR-66979; FK-317; 5-FU; FUDR; FdUMP; Hydroxyurea; Docetaxel; discodermolide; epothilones; vincristine; vinblastine; vinorelbine; meta-pac; irinotecan; SN-38; 10-OH campto; topotecan; etoposide; adriamycin; flavopiridol; Cis-Pt; carbo-Pt; bleomycin; mitomycin C; mithramycin; capecitabine; cytarabine; 2-C1-2′deoxyadenosine; Fludarabine-PO₄; mitoxantrone; mitozolomide; Pentostatin; and Tomudex. One particularly preferred class of anticancer agents are taxanes (e.g., paclitaxel and docetaxel). Another important category of anticancer agent is annonaceous acetogenin.

In some embodiments, the composition is co-administered with a pain relief agent. In some embodiments, the pain relief agents include, but are not limited to, analgesic drugs, anxiolytic drugs, anesthetic drugs, antipsychotic drugs, hypnotic drugs, sedative drugs, and muscle relaxant drugs.

In some embodiments, the analgesic drugs include, but are not limited to, non-steroidal anti-inflammatory drugs, COX-2 inhibitors, and opiates. In some embodiments, the non-steroidal anti-inflammatory drugs are selected from the group consisting of Acetylsalicylic acid (Aspirin), Amoxiprin, Benorylate/Benorilate, Choline magnesium salicylate, Diflunisal, Ethenzamide, Faislamine, Methyl salicylate, Magnesium salicylate, Salicyl salicylate, Salicylamide, arylalkanoic acids, Diclofenac, Aceclofenac, Acemethacin, Alclofenac, Bromfenac, Etodolac, Indometacin, Nabumetone, Oxametacin, Proglumetacin, Sulindac, Tolmetin, 2-arylpropionic acids, Ibuprofen, Alminoprofen, Benoxaprofen, Carprofen, Dexibuprofen, Dexketoprofen, Fenbufen, Fenoprofen, Flunoxaprofen, Flurbiprofen, Ibuproxam, Indoprofen, Ketoprofen, Ketorolac, Loxoprofen, Naproxen, Oxaprozin, Pirprofen, Suprofen, Tiaprofenic acid), N-arylanthranilic acids, Mefenamic acid, Flufenamic acid, Meclofenamic acid, Tolfenamic acid, pyrazolidine derivatives, Phenylbutazone, Ampyrone, Azapropazone, Clofezone, Kebuzone, Metamizole, Mofebutazone, Oxyphenbutazone, Phenazone, Sulfinpyrazone, oxicams, Piroxicam, Droxicam, Lornoxicam, Meloxicam, Tenoxicam, sulphonanilides, nimesulide, licofelone, and omega-3 fatty acids. In some embodiments, the COX-2 inhibitors are selected from the group consisting of Celecoxib, Etoricoxib, Lumiracoxib, Parecoxib, Rofecoxib, and Valdecoxib. In some embodiments, the opiate drugs are selected from the group consisting of natural opiates, alkaloids, morphine, codeine, thebaine, semi-synthetic opiates, hydromorphone, hydrocodone, oxycodone, oxymorphone, desomorphine, diacetylmorphine (Heroin), nicomorphine, dipropanoylmorphine, diamorphine, benzylmorphine, Buprenorphine, Nalbuphine, Pentazocine, meperidine, diamorphine, ethylmorphine, fully synthetic opioids, fentanyl, pethidine, Oxycodone, Oxymorphone, methadone, tramadol, Butorphanol, Levorphanol, propoxyphene, endogenous opioid peptides, endorphins, enkephalins, dynorphins, and endomorphins.

In some embodiments, the anxiolytic drugs include, but are not limited to, benzodiazepines, alprazolam, bromazepam (Lexotan), chlordiazepoxide (Librium), Clobazam, Clonazepam, Clorazepate, Diazepam, Midazolam, Lorazepam, Nitrazepam, temazepam, nimetazepam, Estazolam, Flunitrazepam, oxazepam (Serax), temazepam (Restoril, Normison, Planum, Tenox, and Temaze, Triazolam, serotonin 1A agonists, Buspirone (BuSpar), barbituates, amobarbital (Amytal), pentobarbital (Nembutal), secobarbital (Seconal), Phenobarbital, Methohexital, Thiopental, Methylphenobarbital, Metharbital, Barbexaclone), hydroxyzine, cannabidiol, valerian, kava (Kava Kava), chamomile, Kratom, Blue Lotus extracts, Sceletium tortuosum (kanna) and bacopa monniera.

In some embodiments, the anesthetic drugs include, but are not limited to, local anesthetics, procaine, amethocaine, cocaine, lidocaine, prilocaine, bupivacaine, levobupivacaine, ropivacaine, dibucaine, inhaled anesthetics, Desflurane, Enflurane, Halothanc, Isoflurane, Nitrous oxide, Sevoflurane, Xenon, intravenous anesthetics, Barbiturates, amobarbital (Amytal), pentobarbital (Nembutal), secobarbital (Seconal), Phenobarbital, Methohexital, Thiopental, Methylphenobarbital, Metharbital, Barbexaclone)), Benzodiazepines, alprazolam, bromazepam (Lexotan), chlordiazepoxide (Librium), Clobazam, Clonazepam, Clorazepate, Diazepam, Midazolam, Lorazepam, Nitrazepam, temazepam, nimetazepam, Estazolam, Flunitrazepam, oxazepam (Serax), temazepam (Restoril, Normison, Planum, Tenox, and Temaze), Triazolam, Etomidate, Ketamine, and Propofol.

In some embodiments, the antipsychotic drugs include, but are not limited to, butyrophenones, haloperidol, phenothiazines, Chlorpromazine (Thorazine), Fluphenazine (Prolixin), Perphenazine (Trilafon), Prochlorperazine (Compazine), Thioridazine (Mellaril), Trifluoperazine (Stelazine), Mesoridazine, Promazine, Triflupromazine (Vesprin), Levomepromazine (Nozinan), Promethazine (Phenergan)), thioxanthenes, Chlorprothixene, Flupenthixol (Depixol and Fluanxol), Thiothixene (Navane), Zuclopenthixol (Clopixol & Acuphase)), clozapine, olanzapine, Risperidone (Risperdal), Quetiapine (Seroquel), Ziprasidone (Geodon), Amisulpride (Solian), Paliperidone (Invega), dopamine, bifeprunox, norclozapine (ACP-104), Aripiprazole (Abilify), Tetrabenazine, and Cannabidiol.

In some embodiments, the hypnotic drugs include, but are not limited to, Barbiturates, Opioids, benzodiazepines, alprazolam, bromazepam (Lexotan), chlordiazepoxide (Librium), Clobazam, Clonazepam, Clorazepate, Diazepam, Midazolam, Lorazepam, Nitrazepam, temazepam, nimetazepam, Estazolam, Flunitrazepam, oxazepam (Serax), temazepam (Restoril, Normison, Planum, Tenox, and Temaze), Triazolam, nonbenzodiazepines, Zolpidem, Zaleplon, Zopiclone, Eszopiclone, antihistamines, Diphenhydramine, Doxylamine, Hydroxyzine, Promethazine, gamma-hydroxybutyric acid (Xyrem), Glutethimide, Chloral hydrate, Ethchlorvynol, Levomepromazine, Chlormethiazole, Melatonin, and Alcohol.

In some embodiments, the sedative drugs include, but are not limited to, barbituates, amobarbital (Amytal), pentobarbital (Nembutal), secobarbital (Seconal), Phenobarbital, Methohexital, Thiopental, Methylphenobarbital, Metharbital, Barbexaclone), benzodiazepines, alprazolam, bromazepam (Lexotan), chlordiazepoxide (Librium), Clobazam, Clonazepam, Clorazepate, Diazepam, Midazolam, Lorazepam, Nitrazepam, temazepam, nimetazepam, Estazolam, Flunitrazepam, oxazepam (Serax), temazepam (Restoril, Normison, Planum, Tenox, and Temaze), Triazolam, herbal sedatives, ashwagandha, catnip, kava (Piper methysticum), mandrake, marijuana, valerian, solvent sedatives, chloral hydrate (Noctec), diethyl ether (Ether), ethyl alcohol (alcoholic beverage), methyl trichloride (Chloroform), nonbenzodiazepine sedatives, eszopiclonc (Lunesta), zaleplon (Sonata), zolpidem (Ambien), zopiclone (Imovane, Zimovane)), clomethiazole (clomethiazole), gamma-hydroxybutyrate (GHB), Thalidomide, ethchlorvynol (Placidyl), glutethimide (Doriden), ketamine (Ketalar, Ketaset), methaqualone (Sopor, Quaalude), methyprylon (Noludar), and ramelteon (Rozerem).

In some embodiments, the muscle relaxant drugs include, but are not limited to, depolarizing muscle relaxants, Succinylcholine, short acting non-depolarizing muscle relaxants, Mivacurium, Rapacuronium, intermediate acting non-depolarizing muscle relaxants, Atracurium, Cisatracurium, Rocuronium, Vecuronium, long acting non-depolarizing muscle relaxants, Alcuronium, Doxacurium, Gallamine, Metocurine, Pancuronium, Pipecuronium, and d-Tubocurarine.

In some embodiments, the composition is co-administered with a pain relief agent antagonist. In some embodiments, the pain relief agent antagonists include drugs that counter the effect of a pain relief agent (e.g., an anesthetic antagonist, an analgesic antagonist, a mood stabilizer antagonist, a psycholeptic drug antagonist, a psychoanaleptic drug antagonist, a sedative drug antagonist, a muscle relaxant drug antagonist, and a hypnotic drug antagonist). In some embodiments, pain relief agent antagonists include, but are not limited to, a respiratory stimulant, Doxapram, BIMU-8, CX-546, an opiod receptor antagonist, Naloxone, naltrexone, nalorphine, levallorphan, cyprodime, naltrindole, norbinaltorphimine, buprenorphine, a benzodiazepine antagonist, flumazenil, a non-depolarizing muscle relaxant antagonist, and neostigmine.

### EXAMPLES

The following examples are provided in order to demonstrate and further illustrate certain preferred embodiments and aspects of the present invention and are not to be construed as limiting the scope thereof.

### Example 1

Previous experiments involving dendrimer related technologies are located in U.S. Pat. Nos. 6,471,968, 7,078,461; U.S. patent application Ser. Nos. 09/940,243, 10/431,682, 11,503,742, 11,661,465, 11/523,509, 12/403,179, 12/106,876, 11/827,637, 10/039,393, 10/254,126, 09/867,924, 12/570,977, and 12/645,081; U.S. Provisional Patent Application Ser. Nos. 61/256,699, 61/226,993, 61/140,480, 61/091,608, 61/097,780, 61/101,461, 61/251,244, 60/604,321, 60/690,652, 60/707,991, 60/208,728, 60/718,448, 61/035,949, 60/830,237, and 60/925,181; and International Patent Application Nos. PCT/US2010/051835, PCT/US2010/050893; PCT/US2010/042556, PCT/US2001/015204, PCT/US2005/030278, PCT/US2009/069257, PCT/US2009/036992, PCT/US2009/059071, PCT/US2007/015976, and PCT/US2008/061023.

### Example 2

This example describes the synthesis of modular dendrimer nanoparticles having precise numbers of imaging agents, and the synthesis of antibodies conjugated with modular dendrimer nanoparticles having precise numbers of imaging agents.

A general strategy for synthesis of modular dendrimer nanoparticles having precise numbers of imaging agents and antibody conjugation ligands is shown in scheme 1:

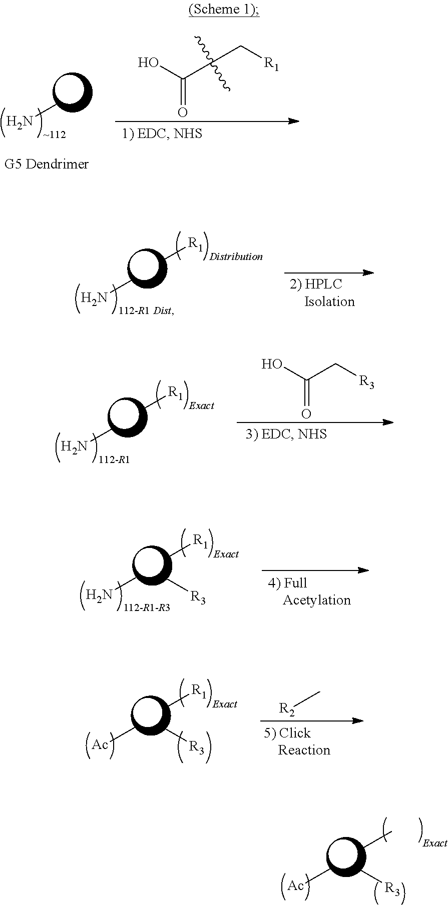

wherein R1 is alkene, thiol, diene, cyclooctyne, fluorinated cyclooctyne, alkyne or azide; wherein R2 is thiol, alkene, dieneophile, azide or alkyne; and R3 is cyclooctyne, fluorinated cyclooctyne, alkyne, alkene, thiol or diene. The dendrimer in this scheme is represented by the circular sphere with the mean number of terminal arms denoted (mean of 112 primary amines per dendrimer for the parent structure). The functional group (e.g., dye molecule, therapeutic agent) is represented with an oval shape.

As shown in Scheme 1, synthesis of the modular dendrimer nanoparticle having a precise number of imaging agents and an antibody conjugation ligand is divided into two sections: 1) isolation of dendrimer with exact numbers of imaging agent conjugation ligands and 2) imaging agent conjugation via the imaging agent conjugation ligands.

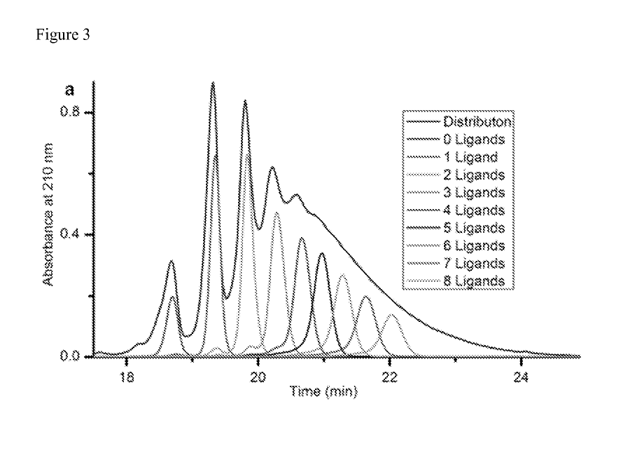

Semi-preparatory HPLC with fractionation is used to isolate dendrimers with exact numbers of alkyne-terminated ligands from stochastically produced dendrimer-ligand conjugates (see, e.g., FIG. 3) (see, e.g., Mullen, D. G.; et al., Chemistry—a European Journal 2010, 16, (35), 10675-10678). The range of isolated dendrimer-ligand species was from 0 to 8 ligands per dendrimer, produced at a minimum of 80% purity. In addition, isolated dendrimer-ligand species have been obtained at scales of tens of mg per batch and applied the isolation technology to ligands with terminal azide, alkene, thiol and cyclooctyne groups.

A strategy for the conjugation of an exact number of imaging agents (e.g., dyes) to the dendrimer is shown in Scheme 2. This process can be divided into two sections: 1) isolation of dendrimers with exact numbers of imaging agent conjugation ligands; and 2) conjugation of imaging agents (e.g., dyes) to dendrimers with exact numbers of imaging agent conjugation ligands. The isolation protocol uses a generation 5 PAMAM dendrimer with alkene-terminated isolation ligands and a gradient elution of water and acetonitrile (with 0.14% trifluoroacetic acid). Fractionation and collection with a semi-preparative HPLC obtains isolated dendrimer particles with exact numbers of isolation ligands per particle (n=1, 2, 3 . . . 9). In a second step, conjugation of a thiol-modified AF488 to the dendrimer with exact numbers of alkynes is based on previously published conditions for UV-catalyzed thiol-ene ‘click’ chemistry (see, e.g., Killops, K. L.; et al., Journal of the American Chemical Society 2008, 130, (15), 5062). An excess of imaging agent (e.g., dye) is used to drive the conversion of the alkene groups. The purity of the PAMAM dendrimer with exact numbers of alkene ligands is assessed by HPLC and ¹H NMR. PAMAM dendrimer with exact numbers of AF488 are also characterized by HPLC and NMR as well as by fluorimetry, and UV-vis.

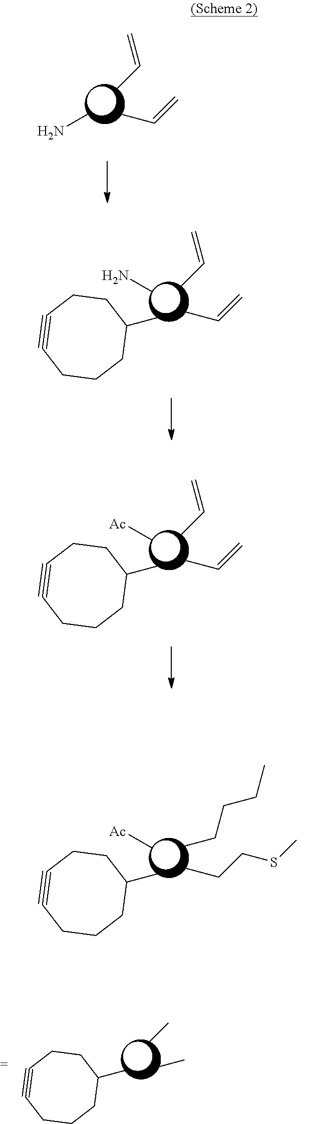

HPLC characterization of the dendimer-imaging agent (e.g., dye) conjugates combined with a peak fitting method (see, e.g., Mullen. D. G.; et al., Acs Nano 2010, 4, (2), 657-670) are used to determine the purity of the dendrimer-dye conjugates. This information is independently confirmed by NMR, fluorimetry, and UV-vis characterization which provide an averaged dye/dendrimer ratio.

A general approach for producing antibodies conjugated with modular dendrimer nanoparticles having exact numbers of imaging agnets (e.g., dye molecules) is shown in Schemes 3 and 4,

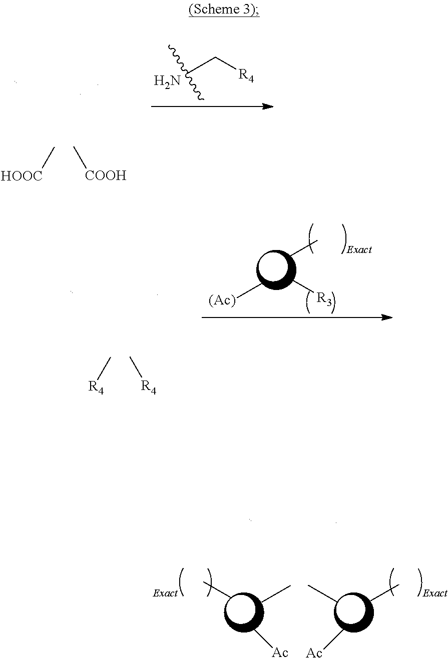

wherein R3 a ligand is configured to facilitate conjugation with another chemical group via click chemistry (e.g., cyclooctyne group, a fluorinated cyclooctyne group, and an alkyne group); wherein R4 is an azide group.

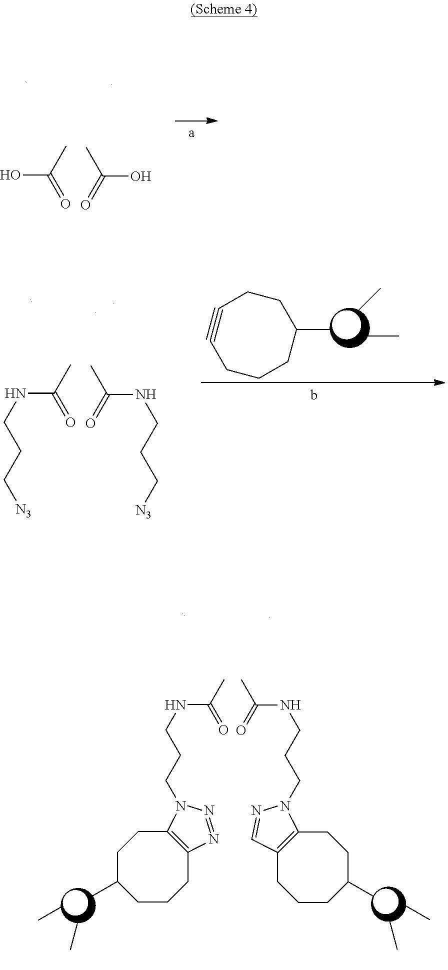

The approach shown in Schemes 3 and 4 is utilized as to precisely control the number and location of conjugated dendrimer and to preserve the specific antigen-binding function of the antibody. The two carboxylic acid groups located at the c-termini of the antibody Fe region are utilized as unique conjugation sites. Although other carboxylic acid groups are present at other regions of the antibody (in aspartic acid and glutamic acid groups and at the c-termini of the hinge region), these groups are considered unreactive either due to post-transcriptional modifications or due to steric blockage. As such, in some embodiments, modification of these c-termini carboxylic acid groups with an azide-terminated linker provides an orthogonal site for controlling antibody-dendrimer conjugation.

Alternative types of orthogonal coupling are shown in Table 2 and include copper catalyzed alkyne-azide ‘click’ reaction. In addition, spacer molecules can be used to reduce imaging agent (e.g., dye molecule) self-quenching.

TABLE 2
Examples of R groups.

| Design # | R1 | R2 | R3 | R4 |
|---|---|---|---|---|
| 1 | Alkene 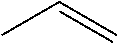 | Thiol 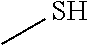 | Cyclooctyne 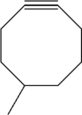 | Azide 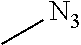 |
| 2 | Alkene 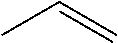 | Thiol 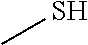 | Fluorinated Cycooctyne 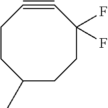 | Azide 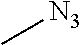 |
| 3 | Alkene 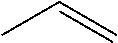 | Thiol 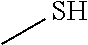 | Alkyne 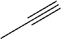 | Azide 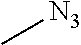 |
| 4 | Thiol 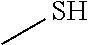 | Alkene 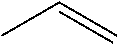 | Cyclooctyne 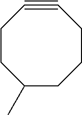 | Azide 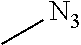 |
| 5 | Thiol 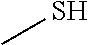 | Alkene 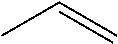 | Fluorinated Cycooctyne 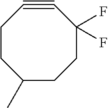 | Azide 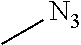 |
| 6 | Thiol 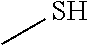 | Alkene 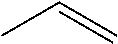 | Alkyne 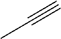 | Azide 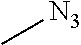 |
| 7 | Diene 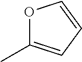 | Dienophile 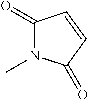 | Cyclooctyne 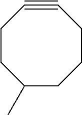 | Azide 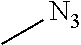 |
| 8 | Diene 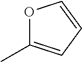 | Dienophile 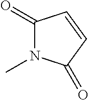 | Fluorinated Cycooctyne 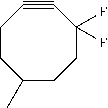 | Azide 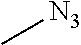 |
| 9 | Diene 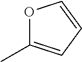 | Dienophile 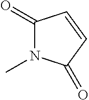 | Alkyne 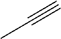 | Azide 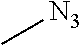 |

### Example 3

This example the synthesis of an antibody conjugated with two modular dendrimer nanoparticles having precise numbers of imaging agents.

A monocolonal anti-CD4 antibody is used in this example. The monoclonal antibody is modified with an azido-amine linker using TSTU-mediated coupling chemistry. To avoid side-reactions with the antibody primary amines, a 1000 fold excess of the azido-amine linker is used. Unreacted linker and coupling agents are removed using a size exclusion column and conjugation of the dendrimer to the antibody is achieved using ring-strain promoted ‘click’ chemistry. The antibody-dye ratio is determined by fluorimetry and UV-vis. Purity of the conjugate is determined by SDS-PAGE and identification of the antibody conjugation region is determined by a fragmentation method (see, e.g., Pierce FAB Preparation Kit-44985. Pierce Biotechnology Product Instructions 2011). Specificity of the antibodies conjugated with two modular dendrimer nanoparticles having precise numbers of imaging agents is determined by flow cytometry with a co-culture of CD4+ and − cells. Batch consistency is measured using fluorimetry and the flow cytometry assay with CD4+/− cells. Finally, the quantitative differences between antibody conjugates with 2, 4, and 6 AF488 dyes is determined by fluorimetry and the flow cytometry assay with with CD4+/− cells. In each characterization of the antibodies conjugated with two modular dendrimer nanoparticles having precise numbers of imaging agents, classically-labeled antibodies serve as controls.

### Example 5

This example describes the synthesis of modular dendrimer nanoparticles having precise numbers of imaging agents, and the synthesis of antibodies conjugated with modular dendrimer nanoparticles having precise numbers of imaging agents.

A general strategy for synthesis of modular dendrimer nanoparticles having precise numbers of imaging agents and antibody conjugation ligands is shown in scheme 5:

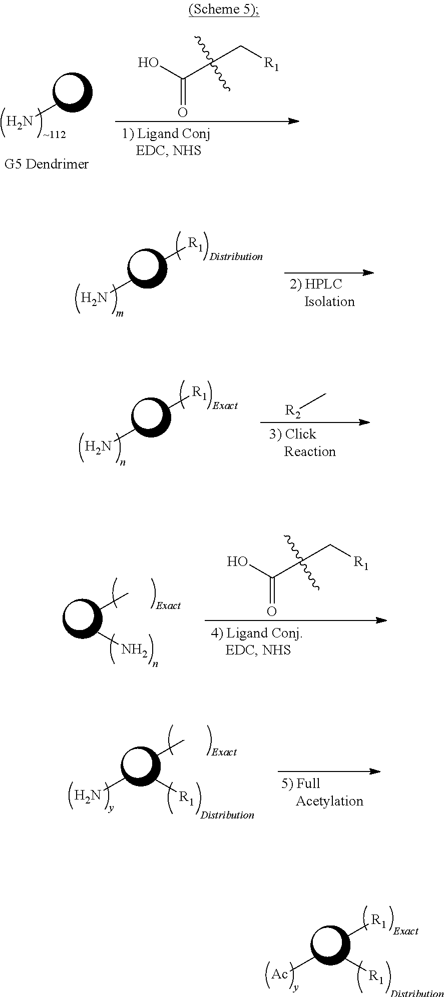

wherein R1 is alkene, thiol, diene, cyclooctyne, fluorinated cyclooctyne, alkyne or azide; and wherein R2 is thiol, alkene, dieneophile, azide, alkyne, cyclooctyne or fluorinated cyclooctyne.

As shown in Scheme 5, synthesis of the modular dendrimer nanoparticle having a precise number of imaging agents and an antibody conjugation ligand is divided into two sections: 1) isolation of dendrimer with exact numbers of imaging agent conjugation ligands and 2) imaging agent conjugation via the imaging agent conjugation ligands.

Semi-preparatory HPLC with fractionation is used to isolate dendrimers with exact numbers of alkyne-terminated ligands from stochastically produced dendrimer-ligand conjugates (see, e.g., FIG. 3) (see, e.g., Mullen, D. G.; et al., Chemistry—a European Journal 2010, 16, (35), 10675-10678). The range of isolated dendrimer-ligand species was from 0 to 8 ligands per dendrimer, produced at a minimum of 80% purity. In addition, isolated dendrimer-ligand species have been obtained at scales of tens of mg per batch and applied the isolation technology to ligands with terminal azide, alkene, thiol and cyclooctyne groups.

A strategy for the conjugation of an exact number of imaging agents (e.g., dyes) to the dendrimer is shown in Scheme 6. This process can be divided into two sections: 1) isolation of dendrimers with exact numbers of imaging agent conjugation ligands; and 2) conjugation of imaging agents (e.g., dyes) to dendrimer with exact numbers of imaging agent conjugation ligands. The isolation protocol uses a generation 5 PAMAM dendrimer with alkene-terminated isolation ligands and a gradient elution of water and acetonitrile (with 0.14% trifluoroacetic acid). Fractionation and collection with a semi-preparative HPLC obtains isolated dendrimer particles with exact numbers of isolation ligands per particle (n=1, 2, 3 . . . 9). In a second step, conjugation of a thiol-modified AF488 to the dendrimer with exact numbers of alkynes is based on previously published conditions for UV-catalyzed thiol-ene ‘click’ chemistry (see. e.g., Killops, K. L.; et al., Journal of the American Chemical Society 2008, 130, (15), 5062). An excess of imaging agent (e.g., dye) is used to drive the conversion of the alkene groups. The purity of the PAMAM dendrimer with exact numbers of alkene ligands is assessed by HPLC and ¹H NMR. PAMAM dendrimer with exact numbers of AF488 are also characterized by HPLC and NMR as well as by fluorimetry, and UV-vis.

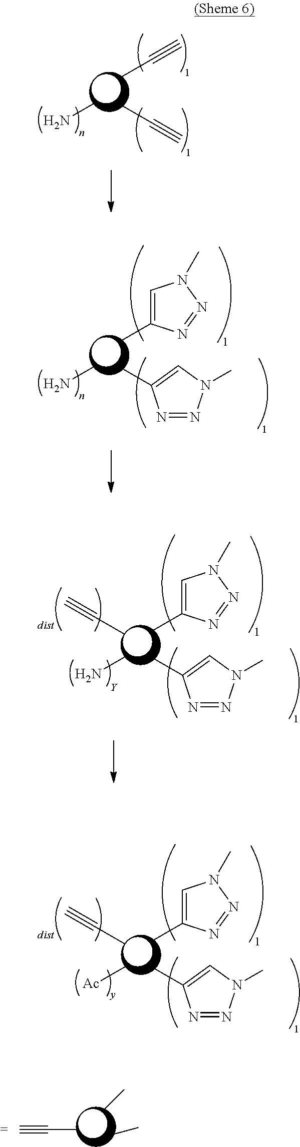

HPLC characterization of the dendimer-imaging agent (e.g., dye) conjugates combined with a peak fitting method (see, e.g., Mullen. D. G.; et al., Acs Nano 2010, 4, (2), 657-670) are used to determine the purity of the dendrimer-dye conjugates. This information is independently confirmed by NMR, fluorimetry, and UV-vis characterization which provide an averaged dye/dendrimer ratio.

A general approach for producing antibodies conjugated with modular dendrimer nanoparticles having exact numbers of imaging agnets (e.g., dye molecules) is shown in Schemes 7 and 8.

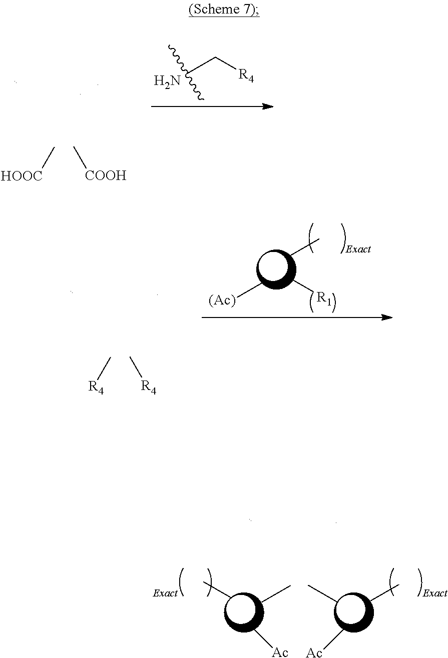

wherein R1 a ligand is configured to facilitate conjugation with another chemical group via click chemistry (e.g., cyclooctyne group, a fluorinated cyclooctyne group, and an alkyne group); wherein R4 is a chemical group that reacts with R1 via a click chemistry reaction.

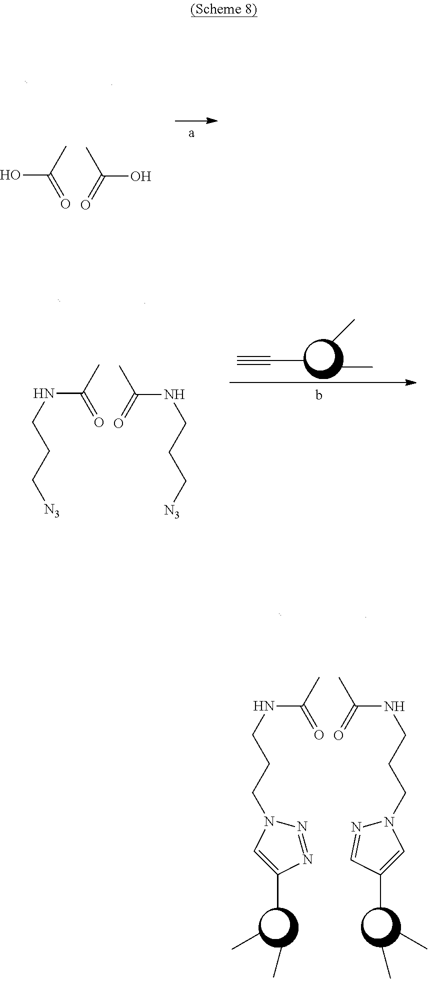

The approach shown in Schemes 7 and 8 is utilized as to precisely control the number and location of conjugated dendrimer and to preserve the specific antigen-binding function of the antibody. The two carboxylic acid groups located at the c-termini of the antibody Fc region are utilized as unique conjugation sites. Although other carboxylic acid groups are present at other regions of the antibody (in aspartic acid and glutamic acid groups and at the c-termini of the hinge region), these groups are considered unreactive either due to post-transcriptional modifications or due to steric blockage. As such, in some embodiments, modification of these c-termini carboxylic acid groups with an azide-terminated linker provides an orthogonal site for controlling antibody-dendrimer conjugation.

Alternative types of orthogonal coupling are shown in Table 3 and include copper catalyzed alkyne-azide ‘click’ reaction. In addition, spacer molecules can be used to reduce imaging agent (e.g., dye molecule) self-quenching.

TABLE 3
Examples of R groups.

| Design # | R1 | R2 | R4 |
|---|---|---|---|
| 1 | Alkene 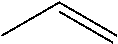 | Thiol 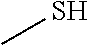 | Thiol 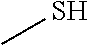 |
| 2 | Thiol 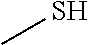 | Alkene 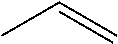 | Alkene 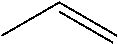 |
| 3 | Diene 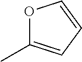 | Dienophile 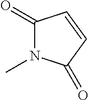 | Dienophile 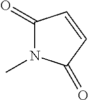 |
| 4 | Alkyne 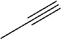 | Azide 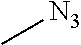 | Azide 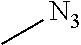 |
| 5 | Cyclooctyne 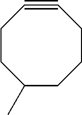 | Azide 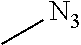 | Azide 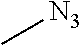 |
| 6 | Fluorinated Cycooctyne 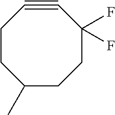 | Azide 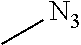 | Azide 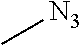 |

### Example 6

This example demonstrates that precisely defined conjugates affect cellular localization and yield unique spectroscopic signal.

Precisely Defined Generation 5 poly(amidoamine) (G5 PAMAM) Dendrimer:Dye samples were prepared using a direct conjugation method of 5-carboxytetramethylrhodamine (TAMRA) and separation of the stochastic material using reverse-phase high performance liquid chromatography (rp-HPLC). The material produced from the column is positively charged with 1-4 numbers of dyes precisely conjugated to the G5 PAMAM dendrimer. The samples were characterized by analytical rp-UPLC, ¹H NMR, MALDI-TOF-MS, emission, and absorption UV-VIS. These samples were incubated with HEK293A cells for 3 hours at a concentration of 0.5 μM in serum free media, and then fixed onto slides. Lifetime studies were conducted in order to determine if the fluorescent dye had a change in lifetime based on number of dye on the dendrimer.

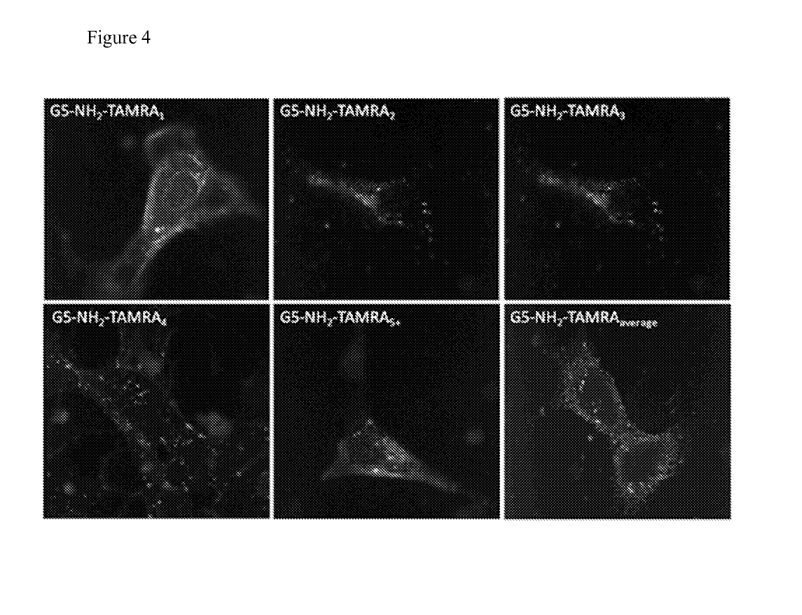

G5-NH₂-TAMRA₁ has a lifetime value of ˜2 ns both in cell and in solution. As TAMRA is conjugated to dendrimer lifetime decreases. G5-NH₂-TAMRA_{1.5(avg)}, the type of conjugate typically employed previously has a lifetime value of ˜1 ns in a cell. The results for all samples are shown in FIG. 4 with lifetimes grey-scale-coded (brighter grey/white 2 ns to grey 1 ns).

The distribution of the polymer-dye conjugate is also dramatically different. G5-TAMRA₁ is diffuse in the cell whereas TAMRA conjugates with multiple dyes, as well as G5-NH₂-TAMRA_{1.5(avg)}, exhibit the more typically observed punctuate distribution. This is remarkable since G5-NH₂-TAMRA_{1.5(avg)}) still contains roughly 34% G5-TAMRA₁ in the mixture, yet its cell distribution is completely different.

In summary, G5-NH₂-TAMRA₁ has unique biodistribution properties, and unique spectroscopic signature, as compared to the rest of the precise ratio conjugates and the typically prepared average conjugate containing distribution of dyes. This is significant because endosomal/lysomal escape is a major consideration for drug/gene delivery.

### INCORPORATION BY REFERENCE

The entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

1-73. (canceled)

74. A composition comprising a plurality of modular dendrimer nanoparticles, wherein approximately 70% of said plurality of modular dendrimer nanoparticles have a precise number of imaging agent conjugation ligands, wherein said imaging agent conjugation ligand is selected from the group consisting of an alkene group, a thiol group, a dieneophile group, and a diene group, wherein said imaging agent conjugation ligand is configured for attachment with attachment ligands complexed with imaging agents, wherein each of said plurality of modular dendrimer nanoparticles further comprise an antibody conjugation ligand, wherein said antibody conjugation ligand is selected from the group consisting of a cyclooctyne group, a fluorinated cyclooctyne group, and an alkyne group.

75. The composition of claim 74, wherein said approximately 70% or more is selected from 75% or higher, 80% or higher, 85% or higher, 90% or higher, 95% or higher, and 99.999% or higher.

76. The composition of claim 74, wherein said antibody conjugation ligand is configured to facilitate conjugation with another chemical group via click chemistry.

77. The composition of claim 74, wherein said imaging agent conjugation ligands are conjugated with imaging agents.

78. The composition of claim 77, wherein said imaging agents are selected from the group consisting of Alexa Fluor 350 (blue), Alexa Fluor 405 (violet), Alexa Fluor 430 (green), Alexa Fluor 488 (cyan-green), Alexa Fluor 500 (green), Alexa Fluor 514 (green), Alexa Fluor 532 (green), Alexa Fluor 546 (yellow), Alexa Fluor 555 (yellow-green), Alexa Fluor 568 (orange), Alexa Fluor 594 (orange-red), Alexa Fluor 610 (red), Alexa Fluor 633 (red), Alexa Fluor 647 (red), Alexa Fluor 660 (red), Alexa Fluor 680 (red), Alexa Fluor 700 (red), Alexa Fluor 750 (red), fluorescein isothiocyanate (FITC), 6-TAMARA, acridine orange, cis-parinaric acid, Hoechst 33342, Brilliant Violet™ 421, BD Horizon™ V450, Pacific Blue™, AmCyan, phycoerythrin (PE), Brilliant Violet™ 605, BD Horizon™ PE-CF594, PI, 7-AAD, allophycocyanin (APC), PE-Cy™ 5, PerCP, PerCP-Cy™ 5.5, PE-Cy™ 7, APC-Cy7, BD APC-H7, Texas Red, Lissamine Rhodamine B, X-Rhodamine, TRITC, Cy2, Cy3, Cy3B, Cy3.5, Cy5.5, Cy7, BODIPY-FL, FluorX™, TruRed, Red 613, NMD, Lucifer yellow, Pacific Orange, Pacific Blue, Cascade Blue, Methoxycoumarin, coumarin, hydroxycoumarin, aminocoumarin, 3-azidocoumarin, DyLight 350, DyLight 405, DyLight 488, DyLight® 550, DyLight 594, DyLight 633, DyLight® 650, DyLight 680, DyLight 755, DyLight 800, Tracy 645, Tracy 652, Atto 488, Atto 520, Atto 532, Atto Rho6G, Atto 550, Atto 565, Atto 590, Atto 594, Atto 633, Atto Rho11, Atto Rho14, Atto 647, Atto 647N, Atto 655, Atto 680, Atto 700, CF™ 350, CF™ 405S, CF™ 405M, CF™ 488A, CF™ 543, CF™ 555, CF™ 568, CF™ 594, CF™ 620R, CF™ 633, CF™ 640R, CF™ 647, CF™ 660, CF™ 660R, CF™ 680, CF™ 680R, CF™ 750, CF™ 770, CF™ 790139La, 141Pr, 142Nd, 143Nd, 144Nd, 145Nd, 146Nd, 147Sm, 148Nd, 149Sm, 150Nd, 151Eu, 152Sm, 153Eu, 154Sm, 156Gd, 158Gd, 159Tb, 160Gd, 162Dy, 164Dy, 165Ho, 166Er, 167Er, 168Er, 169Tm, 170Er, 171Yb, 172Yb, 174Yb, 175Lu, and 176Yb.

79. The composition of claim 74, wherein said antibody conjugation ligand is conjugated with an antibody, wherein said antibody is a monoclonal antibody or a polyclonal antibody.

80. The composition of claim 79, wherein said conjugation with an antibody is at the Fc region of said antibody.

81. The composition of claim 79, wherein said conjugation with an antibody occurs via a 1,3-dipolar cycloaddition reaction.

82. The composition of claim 79, wherein said antibody is an antibody selected from the group consisting of the antibodies shown in Table 1 and Table 2.

83. The composition of claim 74, wherein each of said plurality of modular dendrimer nanoparticles are conjugated with one or more additional functional groups selected from the group consisting of therapeutic agents, targeting agents, and trigger agents,
wherein said therapeutic agents are selected from the group consisting of chemotherapeutic agents, anti-oncogenic agents, anti-angiogenic agents, tumor suppressor agents, anti-microbial agents, expression constructs comprising a nucleic acid encoding a therapeutic protein, pain relief agents, pain relief agent antagonists, agents designed to treat arthritis, agents designed to treat inflammatory bowel disease, agents designed to treat an autoimmune disease, and agents designed to treat inflammatory pelvic disease.

84. The composition of claim 74, wherein said dendrimers within said plurality of modular dendrimer nanoparticles have terminal branches, wherein said terminal branches comprise a blocking agent, wherein said blocking agent comprises an acetyl group.

85. A method for generating pluralities of modular dendrimer nanoparticles wherein approximately 70% or more of said pluralities of modular dendrimer nanoparticles have a precise number of imaging agent conjugation ligands, wherein said imaging agent conjugation ligand is selected from the group consisting of an alkene group, a thiol group, a dieneophile group, and a diene group, wherein said imaging agent conjugation ligand is configured for attachment with attachment ligands complexed with imaging agents, comprising:
a) conjugating imaging agent conjugation ligands with a plurality of dendrimer nanoparticles;
b) separating said plurality of dendrimer nanoparticles conjugated with said imaging agent conjugation ligands into pluralities based upon the number of imaging agent conjugation ligands conjugated to said dendrimer nanoparticles, wherein approximately 70% or more of each plurality of modular dendrimer nanoparticles have a precise number of imaging agent conjugation ligands;
c) conjugating an antibody conjugation ligand with one or more of said pluralities of modular dendrimer nanoparticles have a precise number of imaging agent conjugation ligands, wherein said antibody conjugation ligand is selected from the group consisting of a cyclooctyne group, a fluorinated cyclooctyne group, and an alkyne group, wherein said antibody conjugation ligand is configured to facilitate conjugation with another chemical group via click chemistry;
d) conjugating imaging agents with one or more of said pluralities of modular dendrimer nanoparticles having a precise number of imaging agent conjugation ligands, wherein said conjugating occurs between said imaging agents and said imaging agent conjugation ligands; and
e) conjugating two of said modular dendrimer nanoparticles having a precise number of imaging agent conjugation ligands from one or more of said pluralities with an antibody, wherein said conjugation with an antibody is at the Fc region of said antibody, wherein said conjugation with an antibody occurs via a 1,3-dipolar cycloaddition reaction, wherein said antibody is an antibody selected from the group consisting of the antibodies shown in Table 1 and Table 2.

86. The method of claim 85, wherein said imaging agents are selected from the group consisting of Alexa Fluor 350 (blue), Alexa Fluor 405 (violet), Alexa Fluor 430 (green), Alexa Fluor 488 (cyan-green), Alexa Fluor 500 (green), Alexa Fluor 514 (green), Alexa Fluor 532 (green), Alexa Fluor 546 (yellow), Alexa Fluor 555 (yellow-green), Alexa Fluor 568 (orange), Alexa Fluor 594 (orange-red), Alexa Fluor 610 (red), Alexa Fluor 633 (red), Alexa Fluor 647 (red), Alexa Fluor 660 (red), Alexa Fluor 680 (red), Alexa Fluor 700 (red), Alexa Fluor 750 (red), fluorescein isothiocyanate (FITC), 6-TAMARA, acridine orange, cis-parinaric acid, Hoechst 33342, Brilliant Violet™ 421, BD Horizon™ V450, Pacific Blue™, AmCyan, phycoerythrin (PE), Brilliant Violet™ 605, BD Horizon™ PE-CF594, PI, 7-AAD, allophycocyanin (APC), PE-Cy™ 5, PerCP, PerCP-Cy™ 5.5, PE-Cy™ 7, APC-Cy7, BD APC-H7, Texas Red, Lissamine Rhodamine B, X-Rhodamine, TRITC, Cy2, Cy3, Cy3B, Cy3.5, Cy5.5, Cy7, BODIPY-FL, FluorX™, TruRed, Red 613, NMD, Lucifer yellow, Pacific Orange, Pacific Blue, Cascade Blue, Methoxycoumarin, coumarin, hydroxycoumarin, aminocoumarin, 3-azidocoumarin, DyLight 350, DyLight 405, DyLight 488, DyLight® 550, DyLight 594, DyLight 633, DyLight® 650, DyLight 680, DyLight 755, DyLight 800, Tracy 645, Tracy 652, Atto 488, Atto 520, Atto 532, Atto Rho6G, Atto 550, Atto 565, Atto 590, Atto 594, Atto 633, Atto Rho11, Atto Rho14, Atto 647, Atto 647N, Atto 655, Atto 680, Atto 700, CF™ 350, CF™ 405S, CF™ 405M, CF™ 488A, CF™ 543, CF™ 555, CF™ 568, CF™ 594, CF™ 620R, CF™ 633, CF™ 640R, CF™ 647, CF™ 660, CF™ 660R, CF™ 680, CF™ 680R, CF™ 750, CF™ 770, CF™ 790139La, 141Pr, 142Nd, 143Nd, 144Nd, 145Nd, 146Nd, 147Sm, 148Nd, 149Sm, 150Nd, 151Eu, 152Sm, 153Eu, 154Sm, 156Gd, 158Gd, 159Tb, 160Gd, 162Dy, 164Dy, 165Ho, 166Er, 167Er, 168Er, 169Tm, 170Er, 171Yb, 172Yb, 174Yb, 175Lu, and 176Yb.

87. The method of claim 85, wherein said separating comprises:
application of reverse phase HPLC to yield a subpopulation of pluralities based upon the number of imaging agent conjugation ligands conjugated to said dendrimer nanoparticles indicated by a chromatographic trace, and
applying a peak fitting analysis to said chromatographic trace to identify pluralities of modular dendrimer nanoparticles wherein approximately 70% or more of said pluralities of modular dendrimer nanoparticles have a precise number of imaging agent conjugation ligands.

88. The method of claim 87, wherein said reverse phase HPLC is performed using:
silica gel media comprising a carbon moiety, said carbon moiety ranging from C3 to C8;
C5 silica gel media;
a mobile phase for elution of said ligand-conjugated dendrimers, wherein the mobile phase comprises a linear gradient beginning with 100:0 (v/v) water:acetonitrile and ending with 20:80 (v/v) water:acetonitrile;
a mobile phase for elution of said ligand-conjugated dendrimers, wherein the mobile phase comprises a linear gradient beginning with 100:0 (v/v) water:isopropanol and ending with 20:80 (v/v) water:isopropanol,
wherein said gradient is applied at a flow rate of 1 ml/min, or wherein said gradient is applied at a flow rate of 10 ml/min,
wherein said peak fitting analysis is performed using a Gaussian fit with an exponential decay tail.

89. A method of imaging, comprising
administering to a sample one or more compositions having a precise number and kind of imaging agents as recited in claim 1, wherein said antibodies are capable of binding a cell surface antigens associated with said antibodies, and
wherein upon binding with said cell surface antigens associated with said antibodies said imaging agents are detected.

90. The method of claim 89, wherein said sample is a cell sample selected from the group consisting of an in vitro cell sample, an ex vivo cell sample, an in situ cell sample, and an in vivo cell sample.

91. The method of claim 89, wherein said sample is within a living subject.


SEQUENCE LISTING (2 sequences)

SEQ ID NO: 1 - protein, 7 aa, Artificial Sequence
  Synthetic
YEVDGWK

SEQ ID NO: 2 - protein, 12 aa, Artificial Sequence
  Synthetic
HLNILSTLWKYR